SEQUENCE LISTING (207 sequences)

SEQ ID NO: 1 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 141 aa, synthetic construct
MFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 3 - protein, 15 aa, synthetic construct
MAEGEITTFTALTEK

SEQ ID NO: 4 - protein, 5 aa, synthetic construct
ALTEK

SEQ ID NO: 5 - protein, 4 aa, synthetic construct
LTEK

SEQ ID NO: 6-8 - skipped (3 entries)

SEQ ID NO: 9 - protein, 146 aa, synthetic construct
MALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 10 - protein, 145 aa, synthetic construct
MLTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 11 - protein, 144 aa, synthetic construct
MTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 12 - protein, 143 aa, synthetic construct
MEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 13 - protein, 142 aa, synthetic construct
MKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 14 - protein, 146 aa, synthetic construct
MALTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 15 - protein, 145 aa, synthetic construct
MLTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 16 - protein, 144 aa, synthetic construct
MTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 17 - protein, 143 aa, synthetic construct
MEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 18 - protein, 142 aa, synthetic construct
MKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 19 - protein, 145 aa, synthetic construct
ALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 20 - protein, 144 aa, synthetic construct
LTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 21 - protein, 143 aa, synthetic construct
TEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 22 - protein, 142 aa, synthetic construct
EKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 23 - protein, 141 aa, synthetic construct
KFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 24 - protein, 145 aa, synthetic construct
ALTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 25 - protein, 144 aa, synthetic construct
LTEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 26 - protein, 143 aa, synthetic construct
TEKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 27 - protein, 142 aa, synthetic construct
EKFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 28 - protein, 141 aa, synthetic construct
KFNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 29 - protein, 139 aa, synthetic construct
NLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 30 - protein, 138 aa, synthetic construct
LPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 31 - protein, 137 aa, synthetic construct
PPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 32 - protein, 136 aa, synthetic construct
PGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 33 - protein, 139 aa, synthetic construct
NLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 34 - protein, 138 aa, synthetic construct
LPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 35 - protein, 137 aa, synthetic construct
PPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 36 - protein, 136 aa, synthetic construct
PGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 37 - protein, 140 aa, synthetic construct
MNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 38 - protein, 139 aa, synthetic construct
MLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 39 - protein, 138 aa, synthetic construct
MPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 40 - protein, 137 aa, synthetic construct
MPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 41 - protein, 140 aa, synthetic construct
MNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 42 - protein, 139 aa, synthetic construct
MLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 43 - protein, 138 aa, synthetic construct
MPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 44 - protein, 137 aa, synthetic construct
MPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 45 - protein, 145 aa, synthetic construct
MALTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 46 - protein, 144 aa, synthetic construct
MALTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 47 - protein, 144 aa, synthetic construct
MALTEKPPGGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 48 - protein, 142 aa, synthetic construct
MALTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 49 - protein, 145 aa, synthetic construct; identical to SEQ ID NO: 10

SEQ ID NO: 50 - protein, 144 aa, synthetic construct
MLTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 51 - protein, 143 aa, synthetic construct
MLTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 52 - protein, 142 aa, synthetic construct
MLTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 53 - protein, 141 aa, synthetic construct
MLTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 54 - protein, 144 aa, synthetic construct; identical to SEQ ID NO: 11

SEQ ID NO: 55 - protein, 143 aa, synthetic construct
MTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 56 - protein, 142 aa, synthetic construct
MTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 57 - protein, 141 aa, synthetic construct
MTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 58 - protein, 140 aa, synthetic construct
MTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 59 - protein, 143 aa, synthetic construct; identical to SEQ ID NO: 12

SEQ ID NO: 60 - protein, 142 aa, synthetic construct
MEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 61 - protein, 141 aa, synthetic construct
MEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 62 - protein, 140 aa, synthetic construct
MEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 63 - protein, 139 aa, synthetic construct
MEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 64 - protein, 142 aa, synthetic construct; identical to SEQ ID NO: 13

SEQ ID NO: 65 - protein, 141 aa, synthetic construct
MKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 66 - protein, 140 aa, synthetic construct
MKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 67 - protein, 139 aa, synthetic construct
MKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 68 - protein, 138 aa, synthetic construct
MKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 69 - protein, 144 aa, synthetic construct
ALTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 70 - protein, 143 aa, synthetic construct
ALTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 71 - protein, 142 aa, synthetic construct
ALTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 72 - protein, 141 aa, synthetic construct
ALTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 73 - protein, 144 aa, synthetic construct; identical to SEQ ID NO: 20

SEQ ID NO: 74 - protein, 143 aa, synthetic construct
LTEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 75 - protein, 142 aa, synthetic construct
LTEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 76 - protein, 141 aa, synthetic construct
LTEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 77 - protein, 140 aa, synthetic construct
LTEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 78 - protein, 143 aa, synthetic construct; identical to SEQ ID NO: 21

SEQ ID NO: 79 - protein, 142 aa, synthetic construct
TEKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 80 - protein, 141 aa, synthetic construct
TEKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 81 - protein, 140 aa, synthetic construct
TEKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 82 - protein, 139 aa, synthetic construct
TEKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 83 - protein, 142 aa, synthetic construct; identical to SEQ ID NO: 22

SEQ ID NO: 84 - protein, 141 aa, synthetic construct
EKNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 85 - protein, 140 aa, synthetic construct
EKLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 86 - protein, 139 aa, synthetic construct
EKPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 87 - protein, 138 aa, synthetic construct
EKPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 88 - protein, 141 aa, synthetic construct; identical to SEQ ID NO: 23

SEQ ID NO: 89 - protein, 140 aa, synthetic construct
KNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 90 - protein, 139 aa, synthetic construct
KLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 91 - protein, 138 aa, synthetic construct
KPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 92 - protein, 137 aa, synthetic construct
KPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 93 - protein, 144 aa, synthetic construct
ALTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 94 - protein, 143 aa, synthetic construct
ALTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 95 - skipped

SEQ ID NO: 96 - protein, 142 aa, synthetic construct
ALTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 97 - protein, 141 aa, synthetic construct
ALTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 98 - protein, 144 aa, synthetic construct; identical to SEQ ID NO: 25

SEQ ID NO: 99 - protein, 143 aa, synthetic construct
LTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 100 - protein, 142 aa, synthetic construct
LTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 101 - protein, 141 aa, synthetic construct
LTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 102 - protein, 140 aa, synthetic construct
LTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 103 - protein, 143 aa, synthetic construct; identical to SEQ ID NO: 26

SEQ ID NO: 104 - protein, 142 aa, synthetic construct
TEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 105 - protein, 141 aa, synthetic construct
TEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 106 - protein, 140 aa, synthetic construct
TEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 107 - protein, 139 aa, synthetic construct
TEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 108 - protein, 142 aa, synthetic construct; identical to SEQ ID NO: 27

SEQ ID NO: 109 - protein, 141 aa, synthetic construct
EKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 110 - protein, 140 aa, synthetic construct
EKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 111 - protein, 139 aa, synthetic construct
EKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 112 - protein, 138 aa, synthetic construct
EKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 113 - protein, 141 aa, synthetic construct; identical to SEQ ID NO: 28

SEQ ID NO: 114 - protein, 140 aa, synthetic construct
KNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 115 - protein, 139 aa, synthetic construct
KLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 116 - protein, 138 aa, synthetic construct
KPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 117 - protein, 137 aa, synthetic construct
KPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 118 - protein, 146 aa, synthetic construct; identical to SEQ ID NO: 14

SEQ ID NO: 119 - protein, 144 aa, synthetic construct; identical to SEQ ID NO: 46

SEQ ID NO: 120 - protein, 143 aa, synthetic construct
MALTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 121 - protein, 142 aa, synthetic construct
MALTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 122 - protein, 145 aa, synthetic construct; identical to SEQ ID NO: 15

SEQ ID NO: 123 - protein, 144 aa, synthetic construct
MLTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 124 - protein, 143 aa, synthetic construct
MLTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 125 - protein, 142 aa, synthetic construct
MLTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 126 - protein, 141 aa, synthetic construct
MLTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 127 - protein, 144 aa, synthetic construct; identical to SEQ ID NO: 16

SEQ ID NO: 128 - protein, 143 aa, synthetic construct
MTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 129 - protein, 142 aa, synthetic construct
MTEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 130 - protein, 141 aa, synthetic construct
MTEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 131 - protein, 140 aa, synthetic construct
MTEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 132 - protein, 143 aa, synthetic construct; identical to SEQ ID NO: 17

SEQ ID NO: 133 - protein, 142 aa, synthetic construct
MEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 134 - protein, 141 aa, synthetic construct
MEKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 135 - protein, 140 aa, synthetic construct
MEKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 136 - protein, 139 aa, synthetic construct
MEKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 137 - protein, 142 aa, synthetic construct; identical to SEQ ID NO: 18

SEQ ID NO: 138 - protein, 141 aa, synthetic construct
MKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 139 - protein, 140 aa, synthetic construct
MKLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 140 - protein, 139 aa, synthetic construct
MKPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 141 - protein, 138 aa, synthetic construct
MKPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 142 - protein, 140 aa, synthetic construct
  VARIANT 67: note = Any amino acid
FNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 143 - protein, 139 aa, synthetic construct
  VARIANT 66: note = Any amino acid
NLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 144 - protein, 137 aa, synthetic construct
  VARIANT 64: note = Any amino acid
PPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 145 - protein, 136 aa, synthetic construct
  VARIANT 63: note = Any amino acid
PGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 146 - protein, 140 aa, synthetic construct
  VARIANT 67: note = Any amino acid
FNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 147 - protein, 139 aa, synthetic construct
  VARIANT 66: note = Any amino acid
NLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 148 - protein, 137 aa, synthetic construct
  VARIANT 64: note = Any amino acid
PPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 149 - protein, 136 aa, synthetic construct
  VARIANT 63: note = Any amino acid
PGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 150 - protein, 144 aa, synthetic construct
  VARIANT 71: note = Any amino acid
ALTEKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 151 - protein, 143 aa, synthetic construct
  VARIANT 70: note = Any amino acid
ALTEKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 152 - protein, 143 aa, synthetic construct
  VARIANT 70: note = Any amino acid
ALTEKPPGGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 153 - protein, 141 aa, synthetic construct
  VARIANT 68: note = Any amino acid
ALTEKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 154 - protein, 144 aa, synthetic construct
  VARIANT 71: note = Any amino acid
LTEKFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 155 - protein, 143 aa, synthetic construct
  VARIANT 70: note = Any amino acid
LTEKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 156 - protein, 142 aa, synthetic construct
  VARIANT 69: note = Any amino acid
LTEKLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 157 - protein, 141 aa, synthetic construct
  VARIANT 68: note = Any amino acid
LTEKPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 158 - protein, 140 aa, synthetic construct
  VARIANT 67: note = Any amino acid
LTEKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 159 - protein, 143 aa, synthetic construct
  VARIANT 70: note = Any amino acid
TEKFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 160 - protein, 142 aa, synthetic construct
  VARIANT 69: note = Any amino acid
TEKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 161 - protein, 141 aa, synthetic construct
  VARIANT 68: note = Any amino acid
TEKLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 162 - protein, 140 aa, synthetic construct
  VARIANT 67: note = Any amino acid
TEKPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 163 - protein, 139 aa, synthetic construct
  VARIANT 66: note = Any amino acid
TEKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 164 - protein, 142 aa, synthetic construct
  VARIANT 69: note = Any amino acid
EKFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 165 - protein, 141 aa, synthetic construct
  VARIANT 68: note = Any amino acid
EKNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 166 - protein, 140 aa, synthetic construct
  VARIANT 67: note = Any amino acid
EKLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 167 - protein, 139 aa, synthetic construct
  VARIANT 66: note = Any amino acid
EKPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 168 - protein, 138 aa, synthetic construct
  VARIANT 65: note = Any amino acid
EKPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 169 - protein, 141 aa, synthetic construct
  VARIANT 68: note = Any amino acid
KFNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 170 - protein, 140 aa, synthetic construct
  VARIANT 67: note = Any amino acid
KNLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 171 - protein, 139 aa, synthetic construct
  VARIANT 66: note = Any amino acid
KLPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 172 - protein, 138 aa, synthetic construct
  VARIANT 65: note = Any amino acid
KPPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 173 - protein, 137 aa, synthetic construct
  VARIANT 64: note = Any amino acid
KPGNYKKPKLLYCSNGGHFLRILPDGTVDGTRDRSDQHIQLQLSAESVGEVYIKSTETGQYLAXDTDGLLYGSQTPNEECLFLERLEENHYNTYISKKHAEKNWFVGLKKNGSCKRGPRTHYGQKAILFLPLPVSSD

SEQ ID NO: 174 - protein, 140 aa, synthetic construct; identical to SEQ ID NO: 146
  VARIANT 67: note = Any amino acid

SEQ ID NO: 175 - protein, 145 aa, synthetic construct
  VARIANT 72: note = Any amino acid
ALTEKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 176 - protein, 144 aa, synthetic construct
  VARIANT 71: note = Any amino acid
LTEKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 177 - protein, 143 aa, synthetic construct
  VARIANT 70: note = Any amino acid
TEKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 178 - protein, 142 aa, synthetic construct
  VARIANT 69: note = Any amino acid
EKFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 179 - protein, 141 aa, synthetic construct
  VARIANT 68: note = Any amino acid
KFNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 180 - protein, 141 aa, synthetic construct; identical to SEQ ID NO: 179
  VARIANT 68: note = Any amino acid

SEQ ID NO: 181 - protein, 144 aa, synthetic construct
  VARIANT 71: note = Any amino acid
ALTEKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 182 - protein, 143 aa, synthetic construct; identical to SEQ ID NO: 151
  VARIANT 70: note = Any amino acid

SEQ ID NO: 183 - protein, 142 aa, synthetic construct
  VARIANT 69: note = Any amino acid
ALTEKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 184 - protein, 141 aa, synthetic construct
  VARIANT 68: note = Any amino acid
ALTEKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 185 - protein, 144 aa, synthetic construct; identical to SEQ ID NO: 176
  VARIANT 71: note = Any amino acid

SEQ ID NO: 186 - protein, 143 aa, synthetic construct
  VARIANT 70: note = Any amino acid
LTEKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 187 - protein, 142 aa, synthetic construct
  VARIANT 69: note = Any amino acid
LTEKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 188 - protein, 141 aa, synthetic construct
  VARIANT 68: note = Any amino acid
LTEKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 189 - protein, 140 aa, synthetic construct
  VARIANT 67: note = Any amino acid
LTEKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 190 - protein, 143 aa, synthetic construct; identical to SEQ ID NO: 177
  VARIANT 70: note = Any amino acid

SEQ ID NO: 191 - protein, 142 aa, synthetic construct
  VARIANT 69: note = Any amino acid
TEKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 192 - protein, 141 aa, synthetic construct
  VARIANT 68: note = Any amino acid
TEKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 193 - protein, 140 aa, synthetic construct
  VARIANT 67: note = Any amino acid
TEKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 194 - protein, 139 aa, synthetic construct
  VARIANT 66: note = Any amino acid
TEKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 195 - protein, 142 aa, synthetic construct; identical to SEQ ID NO: 178
  VARIANT 69: note = Any amino acid

SEQ ID NO: 196 - protein, 141 aa, synthetic construct
  VARIANT 68: note = Any amino acid
EKNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 197 - protein, 140 aa, synthetic construct
  VARIANT 67: note = Any amino acid
EKLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 198 - protein, 139 aa, synthetic construct
  VARIANT 66: note = Any amino acid
EKPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 199 - protein, 138 aa, synthetic construct
  VARIANT 65: note = Any amino acid
EKPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 200 - protein, 141 aa, synthetic construct; identical to SEQ ID NO: 179
  VARIANT 68: note = Any amino acid

SEQ ID NO: 201 - protein, 140 aa, synthetic construct
  VARIANT 67: note = Any amino acid
KNLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 202 - protein, 139 aa, synthetic construct
  VARIANT 66: note = Any amino acid
KLPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 203 - protein, 138 aa, synthetic construct
  VARIANT 65: note = Any amino acid
KPPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 204 - protein, 137 aa, synthetic construct
  VARIANT 64: note = Any amino acid
KPGNYKKPKLLYSSNGGHFLRILPDGTVDGTRDRSDQHIQLQLSAESVGEVYIKSTETGQYLCXDTDGLLYGSQTPNEECLFLERLEENHYNTYISKKHAEKNWFVGLKKNGSVKRGPRTHYGQKAILFLPLPVSSD

SEQ ID NO: 205 - protein, 140 aa, synthetic construct
FNLPPGNYKKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 206 - protein, 140 aa, synthetic construct
FNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 207 - protein, 145 aa, synthetic construct
MALTEKNLPPGNYKKPKLLYSSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

(19) United States
(12) Patent Application Publication
(10) Pub. No.: US 2026/0265323 A1
(43) Pub. Date: Sep. 10, 2026

(54) RECOMBINANT MODIFIED FIBROBLAST GROWTH FACTORS AND THERAPEUTIC USES THEREOF

(71) Applicant: Trefoil Therapeutics, Inc., San Diego, CA (US)

(72) Inventor: David EVELETH, San Diego, CA (US)

(21) Appl. No.: 19/341,303

(22) Filed: Sep. 26, 2025

Related U.S. Application Data
(63) Continuation of application No. PCT/US2024/021562, filed on Mar. 27, 2024.
(60) Provisional application No. 63/492,570, filed on Mar. 28, 2023.

(51) Int. Cl. C07K 14/50; A61K 38/00; A61P 27/02

(52) U.S. Cl. CPC C07K 14/501; A61P 27/02; A61K 38/00

(57) ABSTRACT

Described herein are modified fibroblast growth factor (FGF) polypeptides, pharmaceutical compositions and medicaments that include such modified FGF polypeptides, and methods of using such modified FGF polypeptides to treat or prevent conditions that benefit from administration of FGFs.

### CROSS-REFERENCE

This application is a continuation of PCT Application No. PCT/US2024/021562, filed on Mar. 27, 2024, which claims benefit of U.S. Provisional Patent Application No. 63/492,570 filed on Mar. 28, 2023, which incorporated herein by reference in its entirety.

### REFERENCE TO SEQUENCE LISTING SUBMITTED ELECTRONICALLY

This application incorporates by reference a Sequence Listing submitted with this application as a text file entitled “Trefoil 45341-713.301.xml” created on Sep. 24, 2025, and having a size of 227,774 bytes.

### BACKGROUND

Descemetorhexis without Endothelial Keratoplasty (DWEK), sometimes referred to as Descemet Stripping Only (DSO), is a procedure for patients with Fuchs Endothelial Corneal Dystrophy (FECD) where the central area of the Descemet's Membrane is surgically removed and endothelial keratoplasty is not performed. The corneal endothelial cells (CECs) in the peripheral area of the cornea migrate and repopulate the central area of the cornea. The potential benefit of DWEK/DSO is that it does not include transplanted tissue, thereby avoiding the associated issues related to tissue availability and possible rejection. Fibroblast growth factor-1 (FGF-1) is the most potent stimulator of the proliferation of CECs known. FGF-1 in its native or wild-type form (wtFGF-1) is, however, susceptible to both proteolysis and cysteine oxidation, creating a very short biological half-life and thus making it unsuitable as a therapy. Despite continuous efforts to accelerate patients' recovery from DWEK/DSO, there remains an unmet need for approaches that overcome the inherent limitations of conventional methods to effectively stimulate the proliferation and migration of CECs.

### SUMMARY

Provided herein is a method of treating or preventing a corneal disease or condition in a subject in need thereof. In some embodiments, the method comprises administering intracamerally to the subject a pharmaceutical composition comprising a therapeutically effective amount of a modified FGF-1 polypeptide having at least 90% sequence identity to the wild-type FGF-1 amino acid sequence of SEQ ID NO: 1 and the mutations of Cys16Ser, Ala66Cys, and Cys117Val. In some embodiments, the corneal disease or condition comprises Descemetorhexis without Endothelial Keratoplasty (DWEK) or Descemet Stripping Only (DSO).

In some embodiments, the modified FGF-1 polypeptide comprises a methionine upstream of the first residue of SEQ ID NO: 1. In some embodiments, the modified FGF-1 polypeptide comprises an amino acid sequence of that is at least about 90% identical to the sequence of SEQ ID NO: 2. In some embodiments, the modified FGF-1 polypeptide comprises an amino acid sequence of that is at least about 95% identical to the sequence of SEQ ID NO: 2. In some embodiments, the modified FGF-1 polypeptide comprises an amino acid sequence of that is at least about 99% identical to the sequence of SEQ ID NO: 2. In some embodiments, the modified FGF-1 polypeptide comprises the amino acid sequence of SEQ ID NO: 2.

In some embodiments, the subject undergoes cataract surgery prior to, currently with, after the administering. In some embodiments, the corneal disease or condition comprises a corneal endothelial dystrophy or a corneal endothelial damage.

In some embodiments, the subject undergoes cataract surgery prior to, currently with, after the administering; and the corneal disease or condition comprises a corneal endothelial dystrophy or a corneal endothelial damage.

In some embodiments, the corneal endothelial dystrophy or corneal endothelial damage comprises Fuchs dystrophy, bullous keratopathy, congenital hereditary endothelial dystrophy 1, congenital hereditary endothelial dystrophy 2, posterior polymorphous corneal dystrophy, a corneal endothelial damage secondary to ocular surgery, a corneal endothelial damage secondary to cataract surgery, or a corneal endothelial damage secondary to oxidative stress.

In some embodiments, the subject has a risk factor for corneal endothelial damage induced by ocular surgery. In some embodiments, the risk factor comprises diabetes, a low corneal endothelial cell density, a corneal endothelial dystrophy, a small pupil, a shallow anterior chamber, a mature or brunescent cataract, or combinations thereof.

In some embodiments, the subject undergoes cataract surgery prior to, currently with, after the administering and the corneal disease or condition comprises Fuchs dystrophy. In some embodiments, the DWEK or DSO is performed prior to, concurrently with, or after the cataract surgery. In some embodiments, the corneal endothelial dystrophy or corneal endothelial damage is caused by the cataract surgery. In some embodiments, the corneal endothelial dystrophy or corneal endothelial damage is not caused by cataract surgery.

In some embodiments, the pharmaceutical composition comprises a pharmaceutically acceptable carrier, excipient, or diluent. In some embodiments, the pharmaceutical composition comprises a liquid formulation. In some embodiments, the pharmaceutically acceptable carrier, excipient, or diluent comprises a saline. In some embodiments, the saline comprises a phosphate buffered saline (PBS). In some embodiments, the PBS comprises one or more salts of sodium ion or potassium ion. In some embodiments, the one or more salts comprise sodium chloride, potassium chloride, sodium phosphate monobasic, potassium phosphate monobasic, sodium phosphate di-basic anhydrous, or potassium phosphate di-basic anhydrous. In some embodiments, the one or more salts comprise sodium chloride, potassium phosphate monobasic, and sodium phosphate di-basic anhydrous. In some embodiments, the pharmaceutically acceptable carrier, excipient, or diluent comprises a surfactant. In some embodiments, the surfactant comprises a polysorbate, polyoxyl stearate 40, polyoxyethylene hydrogenated castor oil, or an equivalent thereof. In some embodiments, the polysorbate comprises polysorbate 20, polysorbate 40, polysorbate 60, or polysorbate 80. In some embodiments, the polysorbate comprises polysorbate 80. In some embodiments, the pharmaceutically acceptable carrier, excipient, or diluent comprises an albumin. In some embodiments, the albumin comprises a human serum albumin. In some embodiments, the albumin comprises a recombinant human serum albumin. In some embodiments, the pharmaceutically acceptable carrier, excipient, or diluent comprises: (i) PBS comprising sodium chloride, potassium phosphate monobasic, and sodium phosphate di-basic anhydrous; (ii) polysorbate 80, and (iii) a recombinant human serum albumin.

In some embodiments, concentration of the polysorbate is at least about 0.001% (w/v), 0.005% (w/v), 0.01% (w/v), 0.05% (w/v), or 0.1% (w/v). In some embodiments, concentration of the polysorbate is about 0.01% (w/v).

In some embodiments, the pharmaceutical composition comprises at least about 0.1 ng/μl, at least about 0.5 ng/μl, at least about 1 ng/μl, at least about 5 ng/μl, at least about 10 ng/μl of the modified FGF-1 polypeptide. In some embodiments, the pharmaceutical composition comprises about 1 ng/μl of the modified FGF-1 polypeptide.

In some embodiments, the pharmaceutical composition comprises a pH of about 7.2 to about 7.6. In some embodiments, the pharmaceutical composition does not comprise heparin.

In some embodiments, the administering comprises administering a dose of at least about 1 ng, at least about 3 ng, at least about 5 ng, at least about 10 ng, at least about 30 ng, at least about 50 ng, at least about 100 ng, at least about 150 ng, or at least about 200 ng of the modified FGF-1 polypeptide. In some embodiments, the administering comprises administering a dose of at most about 1 ng, at most about 3 ng, at most about 5 ng, at most about 10 ng, at most about 30 ng, at most about 50 ng, at most about 100 ng, at most about 150 ng, or at most about 200 ng of the modified FGF-1 polypeptide. In some embodiments, the administering comprises administering a dose of about 1 ng, about 3 ng, about 5 ng, about 10 ng, about 30 ng, about 50 ng, about 100 ng, about 150 ng, or about 200 ng of the modified FGF-1 polypeptide.

In some embodiments, the administering comprises administering a first dose and a second dose, wherein the second dose is equal to or lower than the first dose, wherein the first or second dose is at least about 1 ng, 3 ng, 5 ng, 10 ng, 30 ng, 50 ng, 100 ng, 150 ng, or 200 ng of the modified FGF-1 polypeptide. In some embodiments, the administering comprises administering a first dose and a second dose, wherein the second dose is equal to or higher than the first dose, wherein the first or second dose is at least about 1 ng, 3 ng, 5 ng, 10 ng, 30 ng, 50 ng, 100 ng, 150 ng, or 200 ng of the modified FGF-1 polypeptide.

In some embodiments, the administering comprises administering a first dose, a second dose, and a third dose, wherein the second dose is equal to or lower than the first dose, and the third dose is equal to or lower than the second dose, wherein the first, second, or third dose is at least about 1 ng, 3 ng, 5 ng, 10 ng, 30 ng, 50 ng, 100 ng, 150 ng, or 200 ng of the modified FGF-1 polypeptide. In some embodiments, the administering comprises administering a first dose, a second dose, and a third dose, wherein the second dose is equal to or higher than the first dose, and the third dose is equal to or higher than the second dose, wherein the first, second, or third dose is at least about 1 ng, 3 ng, 5 ng, 10 ng, 30 ng, 50 ng, 100 ng, 150 ng, or 200 ng of the modified FGF-1 polypeptide. In some embodiments, the administering comprises administering a first dose, a second dose, a third dose, and a fourth dose, wherein the first, second, third, or fourth dose is at least about 1 ng, 3 ng, 5 ng, 10 ng, 30 ng, 50 ng, 100 ng, 150 ng, or 200 ng of the modified FGF-1 polypeptide. In some embodiments, the administering comprises administering a first dose, a second dose, a third dose, a fourth dose, and a fifth dose, wherein the first, second, third, fourth, or fifth dose is at least about 1 ng, 3 ng, 5 ng, 10 ng, 30 ng, 50 ng, 100 ng, 150 ng, or 200 ng of the modified FGF-1 polypeptide. In some embodiments, the administering comprises administering at least five doses, wherein each of the at least five doses is at least about 1 ng, 3 ng, 5 ng, 10 ng, 30 ng, 50 ng, 100 ng, 150 ng, or 200 ng of the modified FGF-1 polypeptide.

In some embodiments, the administering comprises administering a dose of the modified FGF-1 polypeptide before, concurrently with, or after the procedure of DWEK or DSO.

In some embodiments, the administering comprises administering a dose of the modified FGF-1 polypeptide at least 1 day, 2 days, 3 days, 4 days, 5 days, 6 days, 7 days, 2 weeks, 3 weeks, or 4 weeks before the procedure of DWEK or DSO. In some embodiments, the administering comprises administering a dose of the modified FGF-1 polypeptide at most 1 day, 2 days, 3 days, 4 days, 5 days, 6 days, 7 days, 2 weeks, 3 weeks, or 4 weeks before the procedure of DWEK or DSO. In some embodiments, the administering comprises administering a dose of the modified FGF-1 polypeptide 2-3 days before the procedure of DWEK or DSO. In some embodiments, the administering comprises administering a dose of the modified FGF-1 polypeptide on the same day of the procedure of DWEK or DSO. In some embodiments, the administering comprises administering a dose of the modified FGF-1 polypeptide at least 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the administering comprises administering a dose of the modified FGF-1 polypeptide at most 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the administering comprises administering a dose of the modified FGF-1 polypeptide on a weekly basis at least 1 week, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the administering comprises administering a dose of the modified FGF-1 polypeptide on a weekly basis at most 1 week, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the administering comprises administering a dose of the modified FGF-1 polypeptide on a weekly basis 3 weeks after the procedure of DWEK or DSO. In some embodiments, the administering comprises administering a dose of the modified FGF-1 polypeptide on the 7th, 14th, 21st day after the procedure of DWEK or DSO.

In some embodiments, the administering comprises administering at least about 10 microliter (mcL), 30 mcL, 50 mcL, 100 mcL of the pharmaceutical composition having a concentration of 1 ng/mcL of the modified FGF-1 polypeptide.

In some embodiments, the administering comprises administering one to three times a day. In some embodiments, the administering comprises administering once a day. In some embodiments, the administering comprises administering two times a day. In some embodiments, the administering comprises administering three times a day. In some embodiments, the administering comprises administering one to three times a week.

In some embodiments, the pharmaceutical composition is administered by microneedle.

In some embodiments, the administering comprises administering to one eye. In some embodiments, the administering comprises administering to both eyes.

In some embodiments, the DWEK or DSO is performed in one eye. In some embodiments, the DWEK or DSO is performed in both eyes. In some embodiments, the cataract surgery is performed in one eye. In some embodiments, the cataract surgery is performed in both eyes.

In some embodiments, the administering results in a therapeutic effect comprising one or more selected from the group comprising a reduction in corneal endothelial damage, an increase in migration of corneal endothelial cell, an increase in proliferation of corneal endothelial cell, a reduction in corneal edema, an improvement in Best-Corrected Visual Acuity (BCVA), a decrease in corneal thickness, a decrease in central corneal thickness, an improvement in the measurement of visual function and corneal health status (V-FUCHS), a reduction in intraocular pressure, an improvement of slit lamp examination, an improvement in central corneal endothelial cell count, an improvement in peripheral corneal endothelial cell count, or a reduction in time of achieving any thereof.

In some embodiments, the reduction in corneal endothelial damage comprises a reduction in cell death, a reduction in impairment of function, or both.

In some embodiments, the administering results in a therapeutic effect at most 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the administering. In some embodiments, the administering results in a therapeutic effect at least 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the administering.

In some embodiments, the BCVA is measured using the Early Treatment Diabetic Retinopathy Scale (ETDRS) scoring system that has at least 69 letters and a score of at least 20/40.

In some embodiments, the administering comprises administering to both eyes, and wherein the administering results in a therapeutic effect in both eyes that is similar or same between the two eyes. In some embodiments, the administering comprises administering to both eyes, wherein the administering results in an improvement in BCVA that is similar or same between the two eyes. In some embodiments, the administering results in a dose-dependent therapeutic effect. In some embodiments, the therapeutic effect increases with the dose of the modified FGF-1 polypeptide being administered. In some embodiments, the administering results in a therapeutic effect that is not dose-independent. In some embodiments, the administering results in a therapeutic effect in the subject with or without cataract surgery, wherein the therapeutic effect is similar or same between a subject with cataract surgery and a subject without cataract surgery. In some embodiments, the administering results in an improvement in corneal edema or central corneal thickness in the subject with or without cataract surgery, wherein the improvement in corneal edema or central corneal thickness is similar or same between a subject with cataract surgery and a subject without cataract surgery. In some embodiments, the administering results in an improvement in BCVA. In some embodiments, the administering results in a reduction or removal of corneal edema. In some embodiments, the administering results in an accelerated reduction or removal of corneal edema.

In some embodiments, the method comprises a treatment course of at least 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the method comprises a treatment course of at most 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO.

In some embodiments, the administering comprises administering to both eyes, wherein the administering results in an improvement in BCVA that is similar or same between the two eyes. In some embodiments, the administering results in a dose-dependent therapeutic effect. In some embodiments, the therapeutic effect increases with the dose of the modified FGF-1 polypeptide being administered. In some embodiments, the administering results in a therapeutic effect that is not dose-independent. In some embodiments, the administering results in a therapeutic effect in the subject with or without cataract surgery, wherein the therapeutic effect is similar or same between a subject with cataract surgery and a subject without cataract surgery. In some embodiments, the administering results in an improvement in corneal edema or central corneal thickness in the subject with or without cataract surgery, wherein the improvement in corneal edema or central corneal thickness is similar or same between a subject with cataract surgery and a subject without cataract surgery. In some embodiments, the administering results in an improvement in BCVA. In some embodiments, the administering results in a reduction or removal of corneal edema. In some embodiments, the administering results in an accelerated reduction or removal of corneal edema.

In some embodiments, the method comprises a treatment course of at least 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the method comprises a treatment course of at most 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO.

In some embodiments, the DWEK/DSO comprises a central descemetorhexis of about 4 mm to about 5 mm. In some embodiments, a decrease in the DSO size results in a decrease in the period of time to achieve a therapeutic effect in the subject.

In some embodiments, the therapeutic effect comprises a faster improvement to BCVA 20/40, an accelerated improvement to BCVA 20/40, a reduced period of time to BCVA 20/40, or a reduction in corneal edema.

In some embodiments, the subject has diabetes. In some embodiments, the administering results in a therapeutic effect comprising a reduction in corneal thickness or a reduction in corneal edema.

In some embodiments, the corneal disease or condition comprises Fuchs Endothelial Cell Dystrophy (FECD) with central guttae as a contributing cause of visual symptom. In some embodiments, the visual symptom comprises decreased visual acuity from the cornea, morning blur, or central corneal edema. In some embodiments, the subject has clear peripheral cornea with adequate corneal endothelial cell (CEC) reserve. In some embodiments, the subject does not have sub-epithelial haze or fibrosis. In some embodiments, the subject does not exhibit secondary corneal pathology, refractive surgery in the eye, or extra-ocular inflammation caused by a non-infectious or infectious pathogen in the eye. In some embodiments, the subject is human.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (“FIGURE.” or “FIGURES.” herein) of which:

FIG. 1 shows a complete study treatment schema for Groups 1-4, in accordance with one or more embodiments of the present disclosure.

FIG. 2 shows the clinical study diagram with doses, dosing days and visits for evaluation, in accordance with one or more embodiments of the present disclosure.

FIG. 3 shows intracameral injection entry point, in accordance with one or more embodiments of the present disclosure.

FIG. 4 shows analysis of the proportion of all eyes in group 3 and 4 pooled for eyes that received Descemet Stripping Only (DSO) only (dashed line) or DSO+cataract surgery (solid line) by visit, in accordance with one or more embodiments of the present disclosure.

FIG. 5 shows analysis of the proportion of all eyes in group 2 and in groups 3 and 4 pooled for eyes that received DSO only or DSO+cataract surgery by visit, in accordance with one or more embodiments of the present disclosure.

FIG. 6 shows proportion of subjects/eyes with central corneal thickness not more than 10% above baseline (Central Corneal Thickness (CCT) 110% of baseline or less) by visit by group. Differences between group 2 (low dose) and groups 3 and 4 (high dose) are statistically significant with p<0.05 at day 28, Fisher's exact test, in accordance with one or more embodiments of the present disclosure.

FIG. 7 shows proportion of subjects/eyes with central corneal thickness not more than 10% above baseline (CCT 110% of baseline or less) by visit by group. Differences between group 2 (low dose) and groups 3 and 4 (high dose) are statistically significant with p<0.05 at day 28, Fisher's exact test, in accordance with one or more embodiments of the present disclosure.

FIG. 8 shows proportion of subjects/eyes with complete resolution of corneal edema (central corneal thickness at or below baseline) by visit by group. P values, Fisher's exact test, in accordance with one or more embodiments of the present disclosure.

FIG. 9 shows proportion of subjects/eyes with Best-corrected Visual Acuity (BCVA) of 69 letters (20/40) or better by group by visit, all eyes, in accordance with one or more embodiments of the present disclosure.

FIG. 10 shows proportion of subjects/eyes with increase of at 0.6 log MAR (6 lines, 30 letters) BCVA from the first post-surgical visit by group by visit. Comparison of group 2 vs group 3 by Fisher's exact test, in accordance with one or more embodiments of the present disclosure.

FIG. 11 shows mean corneal thickness (+/−SD) for group 2 or groups 3 and 4 pooled by visit. Solid bars include eyes that received DSO only, dotted bars DSO+cataract surgery, in accordance with one or more embodiments of the present disclosure.

FIG. 12 shows mean corneal thickness in group 3 and 4 pooled by visit. Light bars include eyes that received DSO only, dark bars DSO+cataract surgery, in accordance with one or more embodiments of the present disclosure.

FIG. 13 shows proportion of subjects/eyes with complete resolution of corneal edema (central corneal thickness at or below baseline) by visit by group. Solid bars include eyes that received DSO only, dotted bars DSO+cataract surgery, in accordance with one or more embodiments of the present disclosure.

FIG. 14 shows mean corneal thickness in group 2 and groups 3 and 4 pooled by visit. Solid lines, eyes that received DSO only, dotted lines DSO+cataract surgery, in accordance with one or more embodiments of the present disclosure.

FIG. 15 shows mean Early Treatment Diabetic Retinopathy Scale (ETDRS) letter score of first eye and second eye on days 14, 21, 28, 56, and 84, in accordance with one or more embodiments of the present disclosure.

FIG. 16 shows median ETDRS letter score of first eye and second eye on days 14, 21, 28, 56, and 84, in accordance with one or more embodiments of the present disclosure.

FIG. 17 shows analysis of the proportion of first eye (solid line) and second eye (dashed line) for 13 subjects that underwent bilateral DSO with TTHX1114 by BCVA measured using the ETDRS scoring system where ≥69 letters=≥20/40 BCVA, in accordance with one or more embodiments of the present disclosure.

FIG. 18 shows time to 20/40 (weeks) was faster in subjects with smaller DSO (4 mm=4.5, >4.5 to ≤4.5 mm=6.7, ≥5 mm=6.6), in accordance with one or more embodiments of the present disclosure.

FIG. 19 shows time to 20/40 (weeks) was slower in subjects with diabetes (7.7 compared to 5.2 in subjects without diabetes), in accordance with one or more embodiments of the present disclosure.

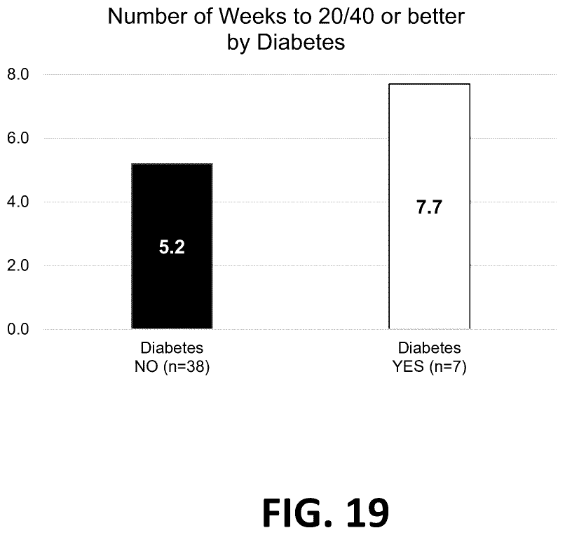

### DETAILED DESCRIPTION

Corneal endothelial hypocellularity driven by corneal endothelial cell (CEC) loss due to trauma, surgical damage, or corneal endothelial dystrophies (CED) can have a significant effect on vision and a high impact on quality of life. Corneal endothelial cell loss or low CEC counts contribute to poor outcomes of ocular surgery, including cataract surgery. Fuchs Endothelial Corneal Dystrophy (FECD) is the most common driver of corneal transplantation. Corneal endothelial hypocellularity secondary to surgery or trauma is an infrequent but significant complication that can lead to corneal transplantation and contributes to visual impairment.

Beyond symptomatic treatment using various pharmacologic therapies, the only treatment option for patients with CED is the transplantation of CECs either as a whole corneal transplant or transplantation of the endothelial layer via a variety of endothelial keratoplasty procedures, including Descemet Stripping Endothelial Keratoplasty (DSEK) and Descemet Membrane Endothelial Keratoplasty (DMEK). Even after transplantation, the number of CECs in the transplant continues to decrease and repeat transplants are sometimes needed.

While the CECs do not repopulate or renew in either normal eyes or in patients with FECD, there is evidence suggesting that cells from the peripheral corneal endothelium can, under some conditions, migrate centrally and that cells of the peripheral corneal endothelium or trabecular meshwork (TM) may contain a progenitor population.

Cataract surgery almost always leads to some degree of CEC loss. In patients with low endothelial cell densities or in patients with other risk factors for CEC loss, cataract surgery can result in loss of large numbers of CECs and loss of CEC function leading to edema and visual loss.

Descemetorhexis without Endothelial Keratoplasty (DWEK), sometimes referred to as Descemet Stripping Only (DSO), is a procedure for patients with FECD where the central area of the Descemet's Membrane is surgically removed (along with FECD-related guttae) and endothelial keratoplasty is not performed. The CECs in the peripheral area of the cornea migrate and repopulate the central area of the cornea. The potential benefit of DWEK/DSO is that it does not include transplanted tissue, thereby avoiding the associated issues related to tissue availability and possible rejection.

Fibroblast growth factors (FGFs) are a large family of proteins that provide protection from insults and cell death and stimulate the proliferation and migration of a wide variety of cells. FGFs are key developmental growth factors in many tissues including the cornea. Fibroblast growth factor-1 (FGF-1) is the most potent stimulator of proliferation of corneal endothelial cells (CECs) known and is the rationale for applying FGF-1 to CED. FGF-1 in its native or wild type form (wtFGF-1) is susceptible to both proteolysis and cysteine oxidation, creating a very short biological half-life and thus making it unsuitable as a therapy.

Provided herein is a pharmaceutical composition comprising a therapeutically effective amount of a modified FGF-1 polypeptide having at least 90% sequence identity to the wild-type FGF-1 amino acid sequence of SEQ ID NO: 1 and the mutations of Cys16Ser, Ala66Cys, and Cys117Val. In some embodiments, the modified FGF-1 polypeptide comprises a methionine upstream of the first residue of SEQ ID NO: 1. In some embodiments, the pharmaceutical composition is for treating or preventing a corneal disease or condition in a subject in need thereof. In some embodiments, the corneal disease or condition comprises Descemetorhexis without Endothelial Keratoplasty (DWEK) or Descemet Stripping Only (DSO).

In some embodiments described herein, where the modified FGF-1 polypeptide is expressed with an N-terminal methionine (N-Met) residue, the polypeptide is subsequently purified without a step requiring proteolytic cleavage for removal of an N-terminal peptide. Accordingly, in some embodiments, the present disclosure provides a modified FGF-1 polypeptide that is prepared by a rapid purification method, without involving a proteolytic cleavage step for removal of an N-terminal peptide. This is particularly advantageous for production of the modified FGF-1 polypeptides per good manufacturing practice (GMP) guidelines. The advantages include the lack of a cleavage step, including eliminating the need for subsequent purification of the cleaved product and removal of the reagents used for cleavage. The further advantage of this is an increase in yield due to decreased handling and the alleviation of the need to test for residual cleavage reagents and contaminants introduced for the cleavage and subsequent separation of cleaved from un-cleaved material.

The modified FGF-1 polypeptides described herein, can have increased stability (e.g. thermostability), reduced number of buried free thiols, and/or increased effective heparan sulfate proteoglycan (HSPG) affinity.

Several other advantages are associated with the use of the modified FGF-1 polypeptides in the methods described herein. For example, the modified FGF-1 polypeptides described herein can be administered without heparin in its pharmaceutical composition or formulation (e.g., an ophthalmic formulation), avoiding potential safety issues related to its biologic origin. In addition, avoidance of heparin allows the use of higher doses of the modified FGF-1 polypeptides without complications resulting from local heparin-induced adverse events or preexisting anti-heparin antibodies. Furthermore, in the absence of heparin, immediate binding of the modified FGF to tissue is maximized and systemic distribution is significantly reduced. The modified FGF-1 polypeptides described herein are also advantage of having enhanced local sequestration and reduced redistribution kinetics, thus increasing the elimination half-life and mean residence time (MRT) at the site of delivery, and allowing for a reduced dosing frequency. This can be the result of modified FGF-1 polypeptides described herein that have increased stability (e.g. thermostability), reduced number of buried free thiols, and/or increased effective heparan sulfate proteoglycan (HSPG) affinity.

The FGF-1 polypeptides of the present disclosure comprise, in various embodiments, modifications at the N-terminus of the polypeptide, such as an addition, a truncation, or a combination of additions and truncations. In some embodiments, the modification is the addition of a single N-terminal methionine residue. In some embodiments, the modification is the addition of an extension peptide. In some embodiments, the modification is a truncation of one or more of the first five residues of FGF-1 polypeptide. In some embodiments, the FGF-1 polypeptides comprise a sequence as set forth in SEQ ID NO: 1, with one or more mutations, in addition to the N-terminal modification.

Several examples of the modified FGF-1 polypeptides disclosed herein comprise an N-terminal methionine (N-Met) residue in a mature form of the polypeptide. The retention of biological activity when amino acids are added to the N-terminus of a protein is unpredictable. Some proteins are tolerant of this, and some are not, and the retention of biological activity and the potential for changes in stability are only determined empirically. The present disclosure identifies that the addition of N-terminal Met residues is tolerated with retention of biological activity and stability.

### Expressed and Mature Forms of the Modified FGF-1 Polypeptides

FGFs stimulate a family seven FGF receptor isoforms, and each FGF stimulates a different pattern of receptors to achieve its specific effect. See, e.g., Ornitz et al. (1996) The Journal of biological chemistry, 1996, 271(25):15292-7; Zhang et al. (2006) The Journal of biological chemistry, 2006, 281(23):15694-700). In some embodiments, modified FGF-1 polypeptide is preferable because it binds to and stimulates all seven FGF receptor isoforms. See Omitz et al. (1996) The Journal of biological chemistry, 1996, 271(25):15292-7.

Embodiments disclosed herein relate to a modified FGF-1 polypeptide or a pharmaceutical composition (e.g., an ophthalmic formulation) comprising a modified FGF-1 polypeptide. Embodiments disclosed herein also relate to a method of treating a chemical or a vesicant injury by administering a modified FGF-1 polypeptide or a pharmaceutical composition (e.g., an ophthalmic formulation) comprising a modified FGF-1 polypeptide. A modified FGF-polypeptide, as used herein, refers to a recombinant FGF that includes a substitution or mutation of one or more different amino acid residues and/or one or more deletions of one or more amino acid residues and/or one or more additions of one or more amino acid residues of SEQ ID NO: 1.

Provided herein, in a first embodiment, is a modified FGF-1 polypeptide comprising the sequence set forth as SEQ ID NO: 1, with one or more mutations, wherein the modified polypeptide further comprises a methionine residue upstream to the first residue of SEQ ID NO: 1. In some embodiments, the modified FGF-1 polypeptide comprising the N-terminal methionine (N-Met) residue is a mature form of the polypeptide. In some instances, the modified FGF-1 polypeptide, according to the first embodiment, comprises one or more mutations at positions 12, 16, 66, 117, and 134 of SEQ ID NO: 1. In some embodiments, the modified FGF-1 polypeptide is expressed in a host cell with a methionine residue upstream to the first residue of SEQ ID NO: 1. In some embodiments, the modified FGF-1 polypeptide is not subject to N-terminal processing for removal of the N-Met residue during maturation. Thus, in some embodiments, the mature form of a modified FGF-1 comprises an N-Met residue and one or more mutations at positions 12, 16, 66, 117, and 134 of SEQ ID NO: 1. An exemplary modified FGF-1 sequence, comprising an N-Met residue, is disclosed as SEQ ID NO: 2.

The present disclosure identifies that a modified FGF-1 as described herein, comprising an N-Met residue in its mature form, has similar biological activity as a version without the N-Met residue. N-terminal methionine removal, or excision, is a co-translational process that occurs as soon as a polypeptide emerges from the ribosome. The removal of the N-terminal methionine involves the substrate specificities of a cleavage enzyme, methionine aminopeptidase (metAP), which recognizes a methionine residue which is followed by an amino acid residue with a small side chain, such as alanine, glycine, proline, serine, threonine, or valine. Due to this substrate sequence specificity, the modified FGF-1 of the first embodiment, which comprises an N-Met residue followed by phenylalanine, see position 1 of SEQ ID NO: 1, is not processed by metAP. Thus, by expressing the modified FGF-1 with a methionine residue directly upstream of SEQ ID NO: 1, a mature modified FGF-1, comprising methionine as its N-terminal residue, can be obtained. In some embodiments, the modified FGF-1 according to the first embodiment is not expressed with an N-terminal peptide and therefore is not subject to proteolytic cleavage for removal of the same, during subsequent purification.

Provided herein, in a second embodiment, is a modified FGF-1 polypeptide comprising the sequence set forth as SEQ ID NO: 1, with one or more mutations, wherein the modified polypeptide further comprises a methionine residue upstream to the first residue of SEQ ID NO: 1, and one or more amino acids of the peptide set forth as SEQ ID NO: 3. A peptide comprising one or more residues of SEQ ID NO: 3 is herein referred to as an “extension peptide.” Thus, the modified FGF-1 according to the second embodiment comprises the sequence set forth as SEQ ID NO: 1, with one or more mutations, a methionine residue upstream to the first residue of SEQ ID NO: 1, and an extension peptide positioned between the methionine residue and the first residue of SEQ ID NO:1. In some embodiments, the modified FGF-1 polypeptide comprising the N-terminal methionine and an extension peptide, positioned between the methionine residue and the first residue of SEQ ID NO: 1, is a mature form of the polypeptide. In some embodiments, the modified FGF-1 polypeptide comprises one or more mutations at positions 12, 16, 66, 117, and 134 of SEQ ID NO: 1, which polypeptide is expressed in a host cell with a methionine residue upstream to the first residue of SEQ ID NO: 1, and further an extension peptide positioned between the methionine residue and the first residue of SEQ ID NO: 1. In some embodiments, the modified FGF-1 polypeptide according to the second embodiment is expressed with an extension peptide comprising five residues of SEQ ID NO: 3, positioned between the methionine residue and the first residue of SEQ ID NO: 1. In some embodiments, the modified FGF-1 polypeptide according to the second embodiment is expressed with four residues of SEQ ID NO: 3, positioned between the methionine residue and the first residue of SEQ ID NO: 1. In some embodiments, the modified FGF-1 polypeptide according to the second embodiment is expressed with three residues of SEQ ID NO: 3, positioned between the methionine residue and the first residue of SEQ ID NO: 1. In some embodiments, the modified FGF-1 polypeptide according to the second embodiment is expressed with two residues of SEQ ID NO: 3, positioned between the methionine residue and the first residue of SEQ ID NO: 1. In some embodiments, the modified FGF-1 polypeptide according to the second embodiment is expressed with one residue of SEQ ID NO: 3, positioned between the methionine residue and the first residue of SEQ ID NO: 1. Exemplary sequences of the extension peptide include SEQ ID NOS: 4-8.

In some instances, the modified FGF-1 polypeptide of the second embodiment, comprising an extension peptide and an N-terminal methionine residue, is not subject to N-terminal processing for removal of the methionine residue, whereas in some instances the methionine is excised by a cleavage enzyme. Typically, the cleavage enzyme is methionine aminopeptidase (metAP). Thus, in some examples, the mature form of the modified FGF-1 polypeptide according to the second embodiment comprises an N-Met residue followed by an extension peptide as described herein. Exemplary sequences of mature forms of modified FGF-1 polypeptides according to the second embodiment, comprising an N-terminal methionine, and one or more residues of the extension peptide, positioned between the methionine residue and the first residue of SEQ ID NO:1, are set forth as SEQ ID NOS: 9-13, wherein the sequences further comprise one or more mutations at amino acids corresponding to positions 12, 16, 66, 117, and 134 of SEQ ID NO: 1. Additional exemplar sequences of mature modified FGF-1 polypeptides comprising an N-terminal methionine, and an extension peptide are set forth as SEQ ID NOS: 14-18. In some other examples, the mature form of the modified FGF-1 polypeptide according to the second embodiment does not comprise an N-Met residue but includes only an extension peptide. Exemplary sequences of mature forms of modified FGF-1 polypeptides according to the second embodiment, comprising an extension peptide, positioned upstream to the first residue of SEQ ID NO:1 are set forth as SEQ ID NOS: 19-23, wherein the sequences further comprise one or more mutations at amino acids corresponding to positions 12, 16, 66, 117, and 134 of SEQ ID NO: 1. Additional exemplar sequences of mature modified FGF-1 polypeptides comprising one or more residues of the extension peptide are set forth as SEQ ID NOS: 24-28. In some embodiments, the methionine residue is cleaved by metAP when the extension peptide starts with an alanine (as in SEQ ID NO: 4) or with a threonine (as in SEQ ID NO: 5). In those instance, the mature FGF-1 polypeptide does not comprise an N-terminal methionine residue, e.g., SEQ ID NOS: 19, 21, 24, and 26.

Provided herein, in a third embodiment, is a modified FGF-1 polypeptide comprising the sequence set forth as SEQ ID NO: 1, with one or more mutations, wherein the modified polypeptide further comprises an extension peptide positioned upstream to the first residue of SEQ ID NO: 1. In some embodiments, the modified FGF-1 polypeptide comprising an extension peptide is a mature form of the polypeptide. In some embodiments, the modified FGF-1 polypeptide comprising one or more mutations at positions 12, 16, 66, 117, and 134 of SEQ ID NO: 1, which polypeptide is expressed in a host cell with one or more amino acid residues of the extension peptide positioned upstream to the first residue of SEQ ID NO: 1. Exemplary sequences of the modified FGF-1 polypeptides comprising an extension peptide, expressed without an N-terminal methionine residue, are set forth as SEQ ID NOS: 19-23, wherein the sequences further comprise one or more mutations at amino acids corresponding to positions 12, 16, 66, 117, and 134 of SEQ ID NO: 1. Additional exemplar sequences of mature modified FGF-1 polypeptides comprising one or more residues of the extension peptide, and expressed without an N-terminal methionine residue, are set forth as SEQ ID NOS: 24-28.

Provided herein, in a fourth embodiment, is a modified FGF-1 polypeptide comprising the sequence set forth as SEQ ID NO: 1, with one or more mutations, wherein the modified polypeptide further comprises a truncation of one or more of the first five residues of SEQ ID NO: 1. In some embodiments, the modified FGF-1 polypeptide comprising the truncation of one or more of the first five residues of SEQ ID NO: 1 is the mature form of the polypeptide. In some embodiments, the modified FGF-1 polypeptide comprises one or more mutations at positions 12, 16, 66, 117, and 134 of SEQ ID NO: 1, wherein one or more of the first five residues of SEQ ID NO: 1 is deleted. In some cases, the modified FGF-1 polypeptide comprising truncations is expressed with an N-terminal methionine residue. For instance, the modified FGF-1 polypeptide, according to the fourth embodiment, can have a sequence wherein the N-Met residue is followed by the second residue, asparagine, of SEQ ID NO: 1. In some cases, the modified FGF-1 polypeptide comprises an N-Met residue followed by the third residue, leucine, of SEQ ID NO: 1. In some cases, the modified FGF-1 polypeptide comprises an N-Met residue followed by the fourth residue, proline, of SEQ ID NO: 1. In some cases, the modified FGF-1 polypeptide comprises an N-Met residue followed by the fifth residue, proline, of SEQ ID NO:1. An extension peptide can be positioned in between the N-Met residue and the first, second, third, fourth, or fifth residue of SEQ ID NO: 1. Examples of a mature form of the modified FGF-1 polypeptide according to the fourth embodiment wherein an N-Met residue is followed by the second, third, fourth, or fifth residue of SEQ ID NO: 1 are shown in SEQ ID NOS: 37-40, wherein the sequences further comprise one or more mutations at amino acids corresponding to positions 12, 16, 66, 117, and 134 of SEQ ID NO: 1. Additional examples of modified FGF-1 polypeptides comprising truncations and an N-Met residue, are provided in SEQ ID NOS: 41-44.

The present disclosure also relates to modified FGF-1 polypeptides comprising one or more mutations of SEQ ID NO: 1, wherein the polypeptides are expressed with an N-Met residue followed by an extension peptide, and the extension peptide is followed by truncation of one or more of the first five residues of SEQ ID NO: 1. In some embodiments, the modified FGF-1 polypeptide comprises one or more mutations at positions 12, 16, 66, 117, and 134 of SEQ ID NO: 1, wherein the polypeptide is expressed with an N-Met residue followed by an extension peptide, and the extension peptide is followed by truncation of one or more of the first five residues of SEQ ID NO: 1. Examples of such sequences expressed with an N-Met residue followed by an extension peptide, which extension peptide is followed by truncation of one or more of the first five residues of SEQ ID NO: 1 are disclosed as SEQ ID NOS: 45-68, wherein the sequences further comprise one or more mutations at amino acids corresponding to positions 12, 16, 66, 117, and 134 of SEQ ID NO: 1. In some examples, the N-terminal methionine is cleaved off by N-terminal processing and accordingly the mature form of the modified FGF-1 polypeptide comprises only one or more residues of the leader fragment followed by truncation of one or more of the first five residues of SEQ ID NO: 1, as exemplified in SEQ ID NOS: 69-92, wherein the exemplary sequences further comprise one or more mutations at amino acids corresponding to positions 12, 16, 66, 117, and 134 of SEQ ID NO: 1. Additional examples of sequences without N-Met residue but including an extension peptide and truncations of N-terminal residues, are provided in SEQ ID NO: 93-117.

In some examples, the N-Met residue is retained in the mature modified FGF-1 polypeptide sequence, and accordingly the mature forms comprise sequences as exemplified in SEQ ID NO: 45-68, further comprising one or more mutations at amino acids corresponding to positions 12, 16, 66, 117, and 134 of SEQ ID NO: 1. Additional examples of sequences comprising an N-Met residue, an extension peptide and truncations of N-terminal residues, are provided in SEQ ID NO: 118-141.

The truncated versions of the modified FGF-1 polypeptides comprising one or more mutations at positions 12, 16, 66, 117, and 134 of SEQ ID NO: 1, are, in a fifth embodiment, expressed without an N-terminal methionine residue, and further without an extension peptide. In some examples, mature modified FGF-1 polypeptides according to the fifth embodiment comprise a sequence as set forth in SEQ ID NOS: 29-32, wherein the sequences further comprise one or more mutations at amino acids corresponding to positions 12, 16, 66, 117, and 134 of SEQ ID NO: 1. In some examples, the modified FGF-1 polypeptides according to the fifth embodiment comprise a sequence selected from the group consisting of SEQ ID NOS: 33-36.

In instances where the modified FGF-1 polypeptide, or its truncated version, comprising one or more mutations at positions 12, 16, 66, 117, and 134 of SEQ ID NO: 1, is expressed with an N-terminal methionine followed by an extension peptide, the methionine residue is either retained or cleaved off of the N-terminus during maturation of the polypeptide after expression. In some examples, where the modified FGF-1 polypeptide is expressed with an alanine next to the N-Met residue, e.g., SEQ ID NO: 14, the methionine is cleaved, to yield a mature FGF-1 polypeptide that does not comprise an N-Met residue, e.g., SEQ ID NO: 19. In some examples, where the modified FGF-1 polypeptide is expressed with a threonine next to the N-Met residue, e.g., SEQ ID NO: 16, the methionine is cleaved, to yield a mature FGF-1 polypeptide that does not comprise an N-Met residue, e.g., SEQ ID NO: 20. In some examples, where the modified FGF-1 polypeptide is expressed with a glutamic acid next to the N-Met residue, e.g., SEQ ID NO: 17, the methionine is not cleaved, to yield a mature FGF-1 that comprise an N-terminal methionine and has the same sequence as the expressed form.

Provided herein, in a sixth embodiment, is a modified FGF-1 polypeptide comprising the sequence set forth as SEQ ID NO: 1, comprising a mutation at position 67. In some embodiments, the modified FGF-1 polypeptide comprises a mutation at position 67 of SEQ ID NO: 1, one or more further mutations at positions 12, 16, 66, 117, and 134, and is expressed with an N-Met residue. The internal methionine at position 67 can be replaced, for example, with an alanine residue. In absence of the internal methionine at position 67, the N-terminal methionine of the modified FGF-1 polypeptide can be cleaved, post-expression; using cyanogen bromide (CNBr), an agent that specifically cleaves the amide bond after methionine residues. In some cases, the modified FGF-1 polypeptides according to the sixth embodiment are expressed with an extension peptide. In some other cases, modified FGF-1 polypeptides according to the sixth embodiment are expressed in a form comprising truncations of one or more of the first five residues of SEQ ID NO: 1, as exemplified in SEQ ID NOS: 142-149, wherein the sequences further comprise one or more mutations at amino acids corresponding to positions 12, 16, 66, 117, and 134 of SEQ ID NO: 1. In yet other examples, the modified FGF-1 polypeptides according to the sixth embodiment are expressed in a form comprising an extension peptide and truncations of one or more of the first five residues of SEQ ID NO: 1, as exemplified in SEQ ID NOS: 151-175. Additional examples of the modified FGF-1 polypeptides according to the sixth embodiment, in their mature forms, are set forth in SEQ ID NOS: 174-204. Among the modified FGF-1 polypeptides expressed in a form that comprises an internal methionine mutation, in cases where the polypeptide is expressed with an N-terminal methionine followed by an alanine or a threonine residue from the extension peptide, e.g., SEQ ID NO: 175 and SEQ ID NO: 177, respectively, the N-terminal methionine can be cleaved off during maturation of the polypeptide either by metAP or using CNBr.

Provided herein, in a seventh embodiment, is a modified FGF-1 polypeptide comprising the sequence set forth as SEQ ID NO: 205, for use in a method as described herein. Provided herein, in an eighth embodiment, is a modified FGF-1 polypeptide comprising the sequence set forth as SEQ ID NO: 206, for use in a method as described herein

The present disclosure further relates to modified FGF-1 polypeptides comprising any combination of deletion, insertion, and substitution of SEQ ID NO: 1, provided that said modified polypeptide comprises one or more mutations of SEQ ID NO: 1. Amino acid substitutions may be introduced into a modified FGF-1 polypeptide and the products screened for a desired activity, e.g., retained/improved effectivity in treating ocular disorders, increased potency in amelioration of Fuch's dystrophy, improved treatment of mustard gas keratopathy. Amino acid substitutions may also be introduced into a modified FGF-1 polypeptide and the products screened for a desired physicochemical property, e.g., less prone to aggregation, improved solubility, prolonged half-life, ease of formulating as an ophthalmic pharmaceutical, enhanced stability, improved shelf-life. Both conservative and non-conservative amino acid substitutions are contemplated.

The modified FGF-1 polypeptide, as in any of the above embodiments, is expressed in a form that comprises at least 136 amino acids. In some embodiments, the modified FGF-1 polypeptide is expressed in a form that comprises 137 amino acids. In some embodiments, the modified FGF-1 polypeptide is expressed in a form that comprises 138 amino acids. In some embodiments, the modified FGF-1 polypeptide is expressed in a form that comprises 139 amino acids. In some embodiments, the modified FGF-1 polypeptide is expressed in a form that comprises 140 amino acids. In some embodiments, the modified FGF-1 polypeptide is expressed in a form that comprises 141 amino acids. In some embodiments, the modified FGF-1 polypeptide is expressed in a form that comprises 142 amino acids. In some embodiments, the modified FGF-1 polypeptide is expressed in a form that comprises 143 amino acids. In some embodiments, the modified FGF-1 polypeptide is expressed in a form that comprises 144 amino acids. In some embodiments, the modified FGF-1 polypeptide is expressed in a form that comprises 145 amino acids. In some embodiments, the modified FGF-1 polypeptide is expressed in a form that comprises 146 amino acids.

The modified FGF-1 polypeptide, as in any of the above embodiments, comprises at least 136 amino acids in the mature form. In some examples, the modified FGF-1 polypeptide comprises 137 amino acids in the mature form. In some examples, the modified FGF-1 polypeptide comprises 138 amino acids in the mature form. In some examples, the modified FGF-1 polypeptide comprises 139 amino acids in the mature form. In some examples, the modified FGF-1 polypeptide comprises 140 amino acids in the mature form. In some examples, the modified FGF-1 polypeptide comprises 141 amino acids in the mature form. In some examples, the modified FGF-1 polypeptide comprises 142 amino acids in the mature form. In some examples, the modified FGF-1 polypeptide comprises 143 amino acids in the mature form. In some examples, the modified FGF-1 polypeptide comprises 144 amino acids in the mature form. In some examples, the modified FGF-1 polypeptide comprises 145 amino acids in the mature form. In some examples, the modified FGF-1 polypeptide comprises 146 amino acids in the mature form.

In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, provided that said polypeptide comprises an N-Met residue in the mature form of the polypeptide. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 9-13, provided that said polypeptide comprises the N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 14-18, provided that said polypeptide comprises the N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 19-23, provided that said polypeptide does not comprise the N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 24-28, provided that said polypeptide does not comprise an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 19-23, provided that said polypeptide does not comprise an N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 37-40, provided that said polypeptide comprises an N-Met residue in its mature form, and the polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 41-44, provided that said polypeptide comprises an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 45-68, provided that said polypeptide comprises one or more mutations at amino acid positions corresponding to positions 12, 16, 66, 117, and 134 of SEQ ID NO: 1, and said polypeptide does not comprise an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 69-92, comprises one or more mutations at amino acid positions corresponding to positions 12, 16, 66, 117, and 134 of SEQ ID NO: 1, and said polypeptide comprises an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 93-117, provided that said polypeptide does not comprise an N-Met residue in its mature form. In some embodiments, the sequence of the modified FGF-1 polypeptide comprises 50%, 55%, 60%, 65%, 70%, 75% 80%, 85%, 86%, 87%, 88%, 89%, 90%, 91%, 92%, 93%, 94%, 95%, 96%, 97%, 98%, 99% or 100% sequence identity to any of the sequences selected from SEQ ID NO: 118-141, provided that said polypeptide comprises an N-Met residue in its mature form. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NOS: 29-32, provided that said polypeptide comprises one or more mutations at amino acid positions corresponding to positions 12, 16, 66, 117, and 134 of SEQ ID NO: 1. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NOS: 33-36.

In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any of the sequences selected from SEQ ID NOS: 142-204.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 12 with, for example, the mutation Lys12Val, and wherein said modified FGF-1 polypeptide comprises an N-terminal methionine in its mature form. In some embodiments, the modified FGF-1 polypeptide comprises a sequence with a mutation at position 12 of SEQ ID NO: 1, for example the mutation Lys12Val, and with truncation of one or more of the first five residue of SEQ ID NO: 1, wherein said modified FGF-1 polypeptide comprises an N-Met residue in its mature form. In some embodiments, the modified FGF-1 polypeptide comprises a sequence with a mutation at position 12 of SEQ ID NO: 1, for example the mutation Lys12Val, with an extension peptide, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at position 12 of SEQ ID NO: 1, for example the mutation Lys12Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 16 with, for example, the mutation Cys16Ser, and wherein said modified FGF-1 polypeptide comprises an N-met residue in its mature form. In some embodiments, the modified FGF-1 polypeptide comprises a sequence with a mutation at position 16 of SEQ ID NO: 1, for example the mutation Cys16Ser, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 16 of SEQ ID NO: 16, for example the mutation Cys16Ser, with an extension peptide, and with truncation of one or more of the first five residue of SEQ ID NO: 1, wherein said modified FGF-1 polypeptide comprises an N-met residue. In some embodiments, the modified FGF-1 polypeptide comprises a sequence with mutations at position 16 of SEQ ID NO: 1, for example the mutation Cys16Ser, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 66 with, for example, the mutation Ala66Cys, and wherein said modified FGF-1 polypeptide comprises an N-terminal methionine in its mature form. In some embodiments, the modified FGF-1 polypeptide comprises a sequence with a mutation at position 66 of SEQ ID NO: 1, for example the mutation Ala66Cys, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 66 of SEQ ID NO: 1, for example the mutation Ala66Cys, with an extension peptide, and with truncation of one or more of the first five residue of SEQ ID NO: 1, wherein said modified FGF-1 polypeptide is expressed with an N-Met residue. In some embodiments, the modified FGF-1 polypeptide comprises a sequence with mutations at position 66 of SEQ ID NO: 1, for example the mutation Ala66Cys, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 117 with, for example, the mutation Cys117Val, and wherein said modified FGF-1 polypeptide comprises an N-met residue in its mature form. In some embodiments, the modified FGF-1 polypeptide comprises a sequence with a mutation at position 117 of SEQ ID NO: 1, for example the mutation Cys117Val, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 117 of SEQ ID NO: 1, for example the mutation Cys117Val, with an extension peptide, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at position 117 of SEQ ID NO: 1, for example the mutation Cys117Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at position 134 with, for example, the mutation Pro134Val, and wherein said modified FGF-1 polypeptide comprises an N-terminal methionine in its mature form. In some embodiments, the modified FGF-1 polypeptide comprises a sequence with a mutation at position 134 of SEQ ID NO: 1, for example the mutation Pro134Val, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with a mutation at position 134 of SEQ ID NO: 1, for example the mutation Pro134Val, with an extension peptide, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at position 134 of SEQ ID NO: 1, for example the mutation Pro134Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at positions 16, 66, and 117 of SEQ ID NO: 1, with, for example, the mutation Cys16Ser, Ala66Cys, and Cys117Val, and wherein said modified FGF-1 polypeptide comprises an N-met residue in its mature form. In some embodiments, the modified FGF-1 polypeptide comprises a sequence with mutations at positions 16, 66, and 117 of SEQ ID NO: 1, with, for example, the mutation Cys16Ser, Ala66Cys, and Cys117Val, and with truncation of one or more of the first five residue of SEQ ID NO: 1, wherein said modified FGF-1 polypeptide comprises an N-met residue in its mature form. In some embodiments, the modified FGF-1 polypeptide comprises a sequence with mutations at positions 16, 66, and 117 of SEQ ID NO: 1, with, for example, the mutation Cys16Ser, Ala66Cys, and Cys117Val, with an extension peptide, and with truncation of one or more of the first five residue of SEQ ID NO: 1, wherein said modified FGF-1 polypeptide comprises an N-met residue. In some embodiments, the modified FGF-1 polypeptide comprises a sequence with mutations at positions 16, 66, and 117 of SEQ ID NO: 1, with, for example, the mutation Cys16Ser, Ala66Cys, and Cys117Val, wherein the polypeptide further comprises a mutation of the methionine at position 67 of SEQ ID NO: 1, and is expressed with a methionine at the N-terminus, which methionine is cleaved off of the polypeptide in its mature form.

In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to a sequence selected from SEQ ID NOs: 2 and 9-204. In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 205 or 206.

In some embodiments, the modified FGF-1 polypeptide is thermostable. As used herein, a thermostable FGF (e.g., a thermostable FGF-1) refers to an FGF having a modified amino acid sequence relative to SEQ ID NO: 1 that is also more stable than the polypeptide of SEQ ID NO: 1 under the same conditions. Examples of mutations capable of conferring thermostability to FGF (e.g., FGF-1) and methods for assessing thermostability are described, for example, in U.S. Pat. Nos. 7,790,682; 7,595,296; 7,696,171; 7,776,825; 7,659,379; 8,119,776; 8,153,770; 8,153,771; and 8,461,111; U.S. Patent Application Publication Nos. 2011/0224404 and 2013/0130983; and in Xia et al. PloS one. (2012) 7(11):e48210. In some embodiments, positions 12 and/or 134 are mutated in FGF-1 to generate a modified FGF-1 that is thermostable.

In some embodiments, the modified FGF-1 polypeptide includes one or more modifications that reduce the number of reactive thiols (e.g., free cysteines). Examples such modifications in FGF-1 are described, for example, in U.S. Pat. Nos. 7,790,682; 7,595,296; 7,696,171; 7,776,825; 7,659,379; 8,119,776; 8,153,770; 8,153,771; and 8,461,111; U.S. Patent Application Publication Nos. 2011/0224404 and 2013/0130983; and in Xia et al. PloS one. (2012) 7(11):e48210. In some embodiments, positions 83 and/or 117 are mutated in SEQ ID NO: 1 to generate a modified FGF-1 that reduces the number of reactive thiols.

In some embodiments, the modified FGF includes one or more modifications that enable formation of an internal disulfide linkage. In some embodiments, position 66 is mutated in SEQ ID NO: 1 to generate a modified FGF-1 that comprises an internal disulfide linkage.

In some embodiments, the modified FGF-1 polypeptides described herein can be administered without exogenous heparin in the formulation for stability, they can be formulated and applied without heparin and thus are more able to bind to the tissue heparans. Such modified FGF-1 polypeptides have a high affinity for tissue heparans that are exposed in a surgical, traumatic or dystrophic conditions and disease-states and so bind to diseased tissue on application. In addition, the modified FGF-1 polypeptides being more thermally stable are suitable for formulation and storage at room temperature. The stability of the modified FGF-1 polypeptides also makes them suitable for administration in both solution (e.g., immediate release) and sustained-release formulations.

In some embodiments, the modified FGF-1 polypeptide is SEQ ID NO: 1 that has been modified at one or more of positions 12, 16, 66, 117, and 134. In some embodiments, the modified FGF is SEQ ID NO: 1 that has been modified at positions 16, 66, and 117. The amino acid positions can be substituted with, e.g., Ser, Cys, Val, or other amino acids to create disulfide linkages between modified amino acids and wild-type amino acids. In some embodiments, the modified FGF comprises the amino acid sequence of SEQ ID NO: 2, also referred to as N-Met THX1114. In some embodiments, the modified FGF-1 polypeptide comprises one or more mutations selected from the group consisting of: Lys12Val, Pro134Val, Ala66Cys, Cys117Val, and Pro134Val. In some embodiments, the modified FGF-1 polypeptide comprises the sequence of SEQ ID NO: 2.

In some embodiments, the modified FGF-1 polypeptides or compositions described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug.

The modified FGF-1 polypeptides described herein may be labeled isotopically (e.g., with a radioisotope) or by other means, including, but not limited to, the use of chromophores or fluorescent moieties, bioluminescent labels, photoactivatable or chemiluminescent labels.

The present discloser further relates to modified FGF polypeptides comprising N-terminal modification(s), wherein the modified FGF polypeptide can be any member of the FGF family, including FGF-1 (SEQ ID NO: 1), FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, and FGF-23, and FGF-24.

In some embodiments, the synthesis of modified FGF-1 polypeptides as described herein is accomplished using means described in the art, using the methods described herein, or by a combination thereof.

In some embodiments, the sequence of the modified FGF comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1 mutated at one or more positions 16, 66, and 117 with, for example, the mutations Cys16Ser, Ala66Cys, and Cys117Val. In some embodiments, the modified FGF comprises the wild-type human FGF-1 sequence with a mutation at positions 16, 66 and 117, for example the mutations Cys16Ser, Ala66Cys, and Cys117Val.

In some embodiments, the sequence of the modified FGF-1 polypeptide comprises 50%, 55%, 60%, 65%, 70%, 75%, 80%, 85%, 86%, 87%, 88%, 89%, 90%, 91%, 92%, 93%, 94%, 95%, 96%, 97%, 98%, 99% or 100% sequence identity to a sequence selected from SEQ ID NOs: 2. In some embodiments, the sequence of the modified FGF-1 polypeptide comprises 50% sequence identity to a sequence selected from SEQ ID NOs: 2. In some embodiments, the sequence of the modified FGF-1 polypeptide comprises 55% sequence identity to a sequence selected from SEQ ID NOs: 2. In some embodiments, the sequence of the modified FGF-1 polypeptide comprises 60% sequence identity to a sequence selected from SEQ ID NOs: 2. In some embodiments, the sequence of the modified FGF-1 polypeptide comprises 65% sequence identity to a sequence selected from SEQ ID NOs: 2. In some embodiments, the sequence of the modified FGF-1 polypeptide comprises 70% sequence identity to a sequence selected from SEQ ID NOs: 2. In some embodiments, the sequence of the modified FGF-1 polypeptide comprises 75% sequence identity to a sequence selected from SEQ ID NOs: 2. In some embodiments, the sequence of the modified FGF-1 polypeptide comprises 80% sequence identity to a sequence selected from SEQ ID NOs: 2. In some embodiments, the sequence of the modified FGF-1 polypeptide comprises 85% sequence identity to a sequence selected from SEQ ID NOs: 2. In some embodiments, the sequence of the modified FGF-1 polypeptide comprises 86% sequence identity to a sequence selected from SEQ ID NOs: 2. In some embodiments, the sequence of the modified FGF-1 polypeptide comprises 87% sequence identity to a sequence selected from SEQ ID NOs: 2. In some embodiments, the sequence of the modified FGF-1 polypeptide comprises 88% sequence identity to a sequence selected from SEQ ID NOs: 2. In some embodiments, the sequence of the modified FGF-1 polypeptide comprises 89% sequence identity to a sequence selected from SEQ ID NOs: 2. In some embodiments, the sequence of the modified FGF-1 polypeptide comprises 90% sequence identity to a sequence selected from SEQ ID NOs: 2. In some embodiments, the sequence of the modified FGF-1 polypeptide comprises 91% sequence identity to a sequence selected from SEQ ID NOs: 2. In some embodiments, the sequence of the modified FGF-1 polypeptide comprises 92% sequence identity to a sequence selected from SEQ ID NOs: 2. In some embodiments, the sequence of the modified FGF-1 polypeptide comprises 93% sequence identity to a sequence selected from SEQ ID NOs: 2. In some embodiments, the sequence of the modified FGF-1 polypeptide comprises 94% sequence identity to a sequence selected from SEQ ID NOs: 2. In some embodiments, the sequence of the modified FGF-1 polypeptide comprises 95% sequence identity to a sequence selected from SEQ ID NOs: 2. In some embodiments, the sequence of the modified FGF-1 polypeptide comprises 96% sequence identity to a sequence selected from SEQ ID NOs: 2. In some embodiments, the sequence of the modified FGF-1 polypeptide comprises 97% sequence identity to a sequence selected from SEQ ID NOs: 2. In some embodiments, the sequence of the modified FGF-1 polypeptide comprises 98% sequence identity to a sequence selected from SEQ ID NOs: 2. In some embodiments, the sequence of the modified FGF-1 polypeptide comprises 99% sequence identity to a sequence selected from SEQ ID NOs: 2. In some embodiments, the sequence of the modified FGF-1 polypeptide comprises 100% sequence identity to a sequence selected from SEQ ID NOs: 2.

### Recombinant Techniques for Preparation of Modified FGF-1 Polypeptides

A variety of host-expression vector systems may be utilized to produce the modified FGF-1 polypeptides provided herein. Such host-expression systems represent vehicles by which the modified FGF-1 polypeptides may be produced and subsequently purified, but also represent cells that may, when transformed or transfected with the appropriate nucleotide coding sequences, exhibit the modified gene product in situ. Examples of host-expression systems include but are not limited to, bacteria, insect, plant, mammalian, including human host systems, such as, but not limited to, insect cell systems infected with recombinant virus expression vectors (e.g., baculovirus) containing nucleotide sequences coding for the modified FGF-1 polypeptides; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing coding sequences for the modified FGF-1 polypeptides; or mammalian cell systems, including human cell systems, e.g., HT1080, COS, CHO, BHK, 293, 3T3, harboring recombinant expression constructs containing promoters derived from the genome of mammalian cells, e.g., metallothionein promoter, or from mammalian viruses, e.g., the adenovirus late promoter; the vaccinia virus 7.5K promoter, or from yeast-derived plasmids e.g., pSH19 and pSH15, or from bacteriophages such as lambda phase and derivatives thereof. Examples of bacterial expression systems include but are not limited to Escherichia coli-derived plasmids (e.g., pBR322, pBR325, pUC12, pUC13, and pET-3); Bacillus subtilis-derived plasmids (e.g. PUB110, pTP5, and pC194).

In some embodiments, a host cell strain is chosen such that it modulates the expression of the inserted sequences, or modifies and processes the gene product in the specific fashion desired. Such modifications and processing of protein products may be important for the function of the protein. Different host cells have specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells, including human host cells, include but are not limited to HT1080, CHO, VERO, BHK, HeLa, COS, MDCK, 293, 3T3, and W138.

For long-term, high-yield production of recombinant peptides, stable expression is desired. For example, cell lines that stably express the recombinant modified FGF-1 polypeptides may be engineered. In some embodiments, rather than using expression vectors that contain viral origins of replication, host cells can be transformed with DNA controlled by appropriate expression control elements, e.g., promoter, enhancer, sequences, transcription terminators, polyadenylation sites, and the like, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. In some examples, this method may advantageously be used to engineer cell lines that express the modified FGF-1 polypeptide product. Such engineered cell lines may be particularly useful in screening and evaluation of compounds that affect the biological of the gene product.

### Disulfide Bond Formation in Modified FGF-1 Polypeptides

In some embodiments, the modified FGF-1 polypeptide of the present disclosure comprises the following mutations in SEQ ID NO: 1—Cys16Ser, Ala66Cys, and Cys117Val, wherein the polypeptide includes an internal disulfide bond between the cysteine residues at positions 66 and 83. For many recombinant proteins, the formation of correct disulfide bonds is vital for attaining their biologically active three-dimensional conformation. The formation of erroneous disulfide bonds can lead to protein misfolding and aggregation into inclusion bodies. In E. coli, cysteine oxidation typically takes places in the periplasm, where disulfide bonds are formed in disulfide exchange reactions catalyzed by a myriad of enzymes, mainly from the Dsb family (Rosano, G. L., & Ceccarelli, E. A. (2014). Recombinant protein expression in Escherichia coli: advances and challenges. Frontiers in Microbiology, 5, 172). By contrast, disulfide bond formation in the cytoplasm is rare. This situation affects the production of recombinant proteins with disulfide bonds that are produced in the cytoplasm, such as a modified FGF-1 polypeptide comprising an internal disulfide linkage between Cys66 and Cys83. Accordingly, in some examples, an engineered E. coli strain that possess an oxidative cytoplasmic environment that favors disulfide bond formation is selected as a host cell for expression of the modified FGF-1 polypeptides (Rosano, G. L., & Ceccarelli, E. A. (2014). Recombinant protein expression in Escherichia coli: advances and challenges. Frontiers in Microbiology, 5, 172). Examples of such strains include but are not limited to Origami (Novagen), which has a trxB-gor-genotype in the K-12 background, and SHuffle® T7 Express strain (NEB), which has a trxB-gor-genotype in a BL21(DE3) background and constitutively expresses a chromosomal copy of the disulfide bond isomerase DsbC. It has been shown that DsbC promotes the correction of mis-oxidized proteins into their correct form and is also a chaperone that can assist in the folding of proteins that do not require disulfide bonds. Without being bound by a particular theory, it is contemplated that due to the action of DsbC, less target protein, such as the modified FGF-1 polypeptide comprising an internal disulfide linkage between Cys66 and Cys83, aggregates into inclusion bodies. Thus, in certain embodiments, the present disclosure identifies an improved method for cytoplasmic production of a modified FGF-1 polypeptide comprising internal disulfide linkage between Cys16 and Cys83.

In some embodiments where the modified FGF-1 polypeptide is expressed with an N-Met residue, the polypeptide is subsequently purified without a step requiring proteolytic cleavage for removal of an N-terminal peptide. Accordingly, in some embodiments, the present disclosure provides a method of rapid purification of the modified FGF-1 polypeptides described herein, without involving a proteolytic cleavage step for removal of an N-terminal peptide. This is particularly advantageous for production of the modified FGF-1 polypeptides per good manufacturing practice (GMP) guidelines. The advantages include the lack of a cleavage step, including eliminating the need for subsequent purification of the cleaved product and removal of the reagents used for cleavage. The further advantage of this is an increase in yield due to decreased handling and the alleviation of the need to test for residual cleavage reagents and contaminants introduced for the cleavage and subsequent separation of cleaved from uncleaved material.

### METHODS OF USE

The present disclosure provides a method of treating or preventing a corneal disease or condition in a subject in need thereof, comprising administering intracamerally to the subject a pharmaceutical composition comprising a therapeutically effective amount of a modified FGF-1 polypeptide having at least 90% sequence identity to the wild-type FGF-1 amino acid sequence of SEQ ID NO: 1 and the mutations of Cys16Ser, Ala66Cys, and Cys117Val, wherein the corneal disease or condition comprises Descemetorhexis without Endothelial Keratoplasty (DWEK) or Descemet Stripping Only (DSO). In some embodiments, the modified FGF-1 polypeptide comprises a methionine upstream of the first residue of SEQ ID NO: 1.

In some embodiments, the sequence of the modified FGF-1 polypeptide comprises 80% sequence identity to a sequence selected from SEQ ID NOs: 2. In some embodiments, the sequence of the modified FGF-1 polypeptide comprises 85% sequence identity to a sequence selected from SEQ ID NOs: 2. In some embodiments, the sequence of the modified FGF-1 polypeptide comprises 86% sequence identity to a sequence selected from SEQ ID NOs: 2. In some embodiments, the sequence of the modified FGF-1 polypeptide comprises 87% sequence identity to a sequence selected from SEQ ID NOs: 2. In some embodiments, the sequence of the modified FGF-1 polypeptide comprises 88% sequence identity to a sequence selected from SEQ ID NOs: 2. In some embodiments, the sequence of the modified FGF-1 polypeptide comprises 89% sequence identity to a sequence selected from SEQ ID NOs: 2. In some embodiments, the sequence of the modified FGF-1 polypeptide comprises 90% sequence identity to a sequence selected from SEQ ID NOs: 2. In some embodiments, the sequence of the modified FGF-1 polypeptide comprises 91% sequence identity to a sequence selected from SEQ ID NOs: 2. In some embodiments, the sequence of the modified FGF-1 polypeptide comprises 92% sequence identity to a sequence selected from SEQ ID NOs: 2. In some embodiments, the sequence of the modified FGF-1 polypeptide comprises 93% sequence identity to a sequence selected from SEQ ID NOs: 2. In some embodiments, the sequence of the modified FGF-1 polypeptide comprises 94% sequence identity to a sequence selected from SEQ ID NOs: 2. In some embodiments, the sequence of the modified FGF-1 polypeptide comprises 95% sequence identity to a sequence selected from SEQ ID NOs: 2. In some embodiments, the sequence of the modified FGF-1 polypeptide comprises 96% sequence identity to a sequence selected from SEQ ID NOs: 2. In some embodiments, the sequence of the modified FGF-1 polypeptide comprises 97% sequence identity to a sequence selected from SEQ ID NOs: 2. In some embodiments, the sequence of the modified FGF-1 polypeptide comprises 98% sequence identity to a sequence selected from SEQ ID NOs: 2. In some embodiments, the sequence of the modified FGF-1 polypeptide comprises 99% sequence identity to a sequence selected from SEQ ID NOs: 2. In some embodiments, the sequence of the modified FGF-1 polypeptide comprises 100% sequence identity to a sequence selected from SEQ ID NOs: 2.

In some cases, the subject undergoes cataract surgery prior to, currently with, after the administering. In some cases, the corneal disease or condition comprises a corneal endothelial dystrophy or a corneal endothelial damage.

In some cases, (i) the subject undergoes cataract surgery prior to, currently with, after the administering; and (ii) the corneal disease or condition comprises a corneal endothelial dystrophy or a corneal endothelial damage.

In some embodiments, the corneal endothelial dystrophy or corneal endothelial damage comprises Fuchs dystrophy, bullous keratopathy, congenital hereditary endothelial dystrophy 1, congenital hereditary endothelial dystrophy 2, posterior polymorphous corneal dystrophy, a corneal endothelial damage secondary to ocular surgery, a corneal endothelial damage secondary to cataract surgery, or a corneal endothelial damage secondary to oxidative stress.

In some embodiments, the subject has a risk factor for corneal endothelial damage induced by ocular surgery. In some embodiments, the risk factor comprises diabetes, a low corneal endothelial cell density, a corneal endothelial dystrophy, a small pupil, a shallow anterior chamber, a mature or brunescent cataract, or combinations thereof.

In some embodiments, the subject undergoes cataract surgery prior to the administering and the corneal disease or condition comprises Fuchs dystrophy. In some embodiments, the subject undergoes cataract surgery currently with the administering and the corneal disease or condition comprises Fuchs dystrophy. In some embodiments, the subject undergoes cataract surgery after the administering and the corneal disease or condition comprises Fuchs dystrophy. In some embodiments, the DWEK or DSO is performed prior to the cataract surgery. In some embodiments, the DWEK or DSO is performed concurrently with the cataract surgery. In some embodiments, the DWEK or DSO is performed after the cataract surgery.

In some embodiments, the corneal endothelial dystrophy or corneal endothelial damage is caused by the cataract surgery. In some embodiments, the corneal endothelial dystrophy or corneal endothelial damage is not caused by cataract surgery.

In some embodiments, the pharmaceutical composition comprises a pharmaceutically acceptable carrier, excipient, or diluent. In some embodiments, the pharmaceutical composition comprises a liquid formulation. In some embodiments, the pharmaceutically acceptable carrier, excipient, or diluent comprises a saline. In some embodiments, the saline comprises a phosphate buffered saline (PBS). In some embodiments, the PBS comprises one or more salts of sodium ion or potassium ion. In some embodiments, the one or more salts comprise sodium chloride, potassium chloride, sodium phosphate monobasic, potassium phosphate monobasic, sodium phosphate di-basic anhydrous, or potassium phosphate di-basic anhydrous. In some embodiments, the one or more salts comprise sodium chloride, potassium phosphate monobasic, and sodium phosphate di-basic anhydrous. In some embodiments, the pharmaceutical composition comprises sodium chloride, potassium phosphate monobasic, and sodium phosphate di-basic anhydrous. In some embodiments, the pharmaceutically acceptable carrier, excipient, or diluent comprises sodium chloride, potassium phosphate monobasic, and sodium phosphate di-basic anhydrous.

In some embodiments, the pharmaceutically acceptable carrier, excipient, or diluent comprises a surfactant. In some embodiments, the surfactant comprises a polysorbate, polyoxyl stearate 40, polyoxyethylene hydrogenated castor oil, or an equivalent thereof. In some embodiments, the pharmaceutically acceptable carrier, excipient, or diluent comprises polysorbate. In some embodiments, the polysorbate comprises polysorbate 20, polysorbate 40, polysorbate 60, or polysorbate 80. In some embodiments, the polysorbate comprises polysorbate 20. In some embodiments, the polysorbate comprises polysorbate 40. In some embodiments, the polysorbate comprises polysorbate 60. In some embodiments, the polysorbate comprises polysorbate 80. In some embodiments, the pharmaceutical composition comprises polysorbate 80. In some embodiments, the pharmaceutically acceptable carrier, excipient, or diluent comprises polysorbate 80.

In some embodiments, the pharmaceutically acceptable carrier, excipient, or diluent comprises an albumin. In some embodiments, the albumin comprises a human serum albumin. In some embodiments, the albumin comprises a recombinant human serum albumin. In some embodiments, the pharmaceutical composition comprises a recombinant human serum albumin. In some embodiments, the pharmaceutically acceptable carrier, excipient, or diluent comprises a recombinant human serum albumin.

In some embodiments, the pharmaceutical composition comprises: (i) PBS comprising sodium chloride, potassium phosphate monobasic, and sodium phosphate di-basic anhydrous; (ii) polysorbate 80, and (iii) a recombinant human serum albumin. In some embodiments, the pharmaceutically acceptable carrier, excipient, or diluent comprises: (i) PBS comprising sodium chloride, potassium phosphate monobasic, and sodium phosphate di-basic anhydrous; (ii) polysorbate 80, and (iii) a recombinant human serum albumin.

In some embodiments, concentration (w/v) of the polysorbate is at least about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1%. In some embodiments, concentration (w/v) of the polysorbate is at most about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1%. In some embodiments, concentration (w/v) of the polysorbate is about 0.001% to 0.10%, 0.003% to 0.09%, 0.005% to 0.08%, 0.007% to 0.07%, 0.01% to 0.06%, 0.02% to 0.05%, or 0.03% to 0.04%. In some embodiments, concentration of the polysorbate is about 0.01% (w/v). In some embodiments, concentration (w/v) of the polysorbate 80 is at least about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1%. In some embodiments, concentration (w/v) of the polysorbate 80 is at most about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1%. In some embodiments, concentration (w/v) of the polysorbate 80 is about 0.001% to 0.1%, 0.003% to 0.09%, 0.005% to 0.08%, 0.007% to 0.07%, 0.01% to 0.06%, 0.02% to 0.05%, or 0.03% to 0.04%. In some embodiments, concentration of the polysorbate is about 0.01% (w/v).

In some embodiments, the pharmaceutical composition comprises at least about 0.1 ng/μl, 0.2 ng/μl, 0.3 ng/μl, 0.4 ng/μl, 0.5 ng/μl, 0.6 ng/μl, 0.7 ng/μl, 0.8 ng/μl, 0.9 ng/μl, 1 ng/μl, 2 ng/μl, 3 ng/μl, 4 ng/μl, 5 ng/μl, 6 ng/μl, 7 ng/μl, 8 ng/μl, 9 ng/μl, 10 ng/μl, 12 ng/μl, 15 ng/μl, or 20 ng/μl of the modified FGF-1 polypeptide. In some embodiments, the pharmaceutical composition comprises at most about 0.1 ng/μl, 0.2 ng/μl, 0.3 ng/μl, 0.4 ng/μl, 0.5 ng/μl, 0.6 ng/μl, 0.7 ng/μl, 0.8 ng/μl, 0.9 ng/μl, 1 ng/μl, 2 ng/μl, 3 ng/μl, 4 ng/μl, 5 ng/μl, 6 ng/μl, 7 ng/μl, 8 ng/μl, 9 ng/μl, 10 ng/μl, 12 ng/μl, 15 ng/μl, or 20 ng/μl of the modified FGF-1 polypeptide. In some embodiments, the pharmaceutical composition comprises about 0.1 ng/μl to 20 ng/μl, 0.2 ng/μl to 15 ng/μl, 0.3 ng/μl to 12 ng/μl, 0.4 ng/μl to 10 ng/μl, 0.5 ng/μl to 9 ng/μl, 0.6 ng/μl to 8 ng/μl, 0.7 ng/μl to 7 ng/μl, 0.8 ng/μl to 6 ng/μl, 0.9 ng/μl to 5 ng/μl, 1 ng/μl to 4 ng/μl, or 2 ng/μl to 3 ng/μl of the modified FGF-1 polypeptide. In some embodiments, the pharmaceutical composition comprises about 1 ng/μl of the modified FGF-1 polypeptide.

In some embodiments, a pH of the pharmaceutical composition is at least about 7.0, 7.1, 7.2, 7.3, 7.4, 7.5, 7.6, 7.7, 7.8, 7.9, or 8.0. In some embodiments, a pH of the pharmaceutical composition is at most about 7.0, 7.1, 7.2, 7.3, 7.4, 7.5, 7.6, 7.7, 7.8, 7.9, or 8.0. In some embodiments, a pH of the pharmaceutical composition is about 7.0 to 8.0, 7.1 to 7.9, 7.2 to 7.8, 7.3 to 7.7, 7.4 to 7.6, or 7.2 to 7.6. In some embodiments, a pH of the pharmaceutical composition is about 7.4.

In some embodiments, the pharmaceutical composition does not comprise heparin.

In some embodiments, the administering comprises administering a dose of at least about 1 ng, at least about 2 ng, at least about 3 ng, at least about 4 ng, at least about 5 ng, at least about 6 ng, at least about 7 ng, at least about 8 ng, at least about 9 ng, at least about 10 ng, at least about 12 ng, at least about 15 ng, at least about 18 ng, at least about 20 ng, at least about 30 ng, at least about 40 ng, at least about 50 ng, at least about 60 ng, at least about 70 ng, at least about 80 ng, at least about 90 ng, at least about 100 ng, at least about 120 ng, at least about 150 ng, at least about 180 ng, at least about 200 ng, at least about 300 ng, at least about 400 ng, or at least about 500 ng of the modified FGF-1 polypeptide. In some embodiments, the administering comprises administering a dose of at most about 1 ng, at most about 2 ng, at most about 3 ng, at most about 4 ng, at most about 5 ng, at most about 6 ng, at most about 7 ng, at most about 8 ng, at most about 9 ng, at most about 10 ng, at most about 12 ng, at most about 15 ng, at most about 18 ng, at most about 20 ng, at most about 30 ng, at most about 40 ng, at most about 50 ng, at most about 60 ng, at most about 70 ng, at most about 80 ng, at most about 90 ng, at most about 100 ng, at most about 120 ng, at most about 150 ng, at most about 180 ng, at most about 200 ng, at most about 300 ng, at most about 400 ng, or at most about 500 ng of the modified FGF-1 polypeptide. In some embodiments, the administering comprises administering a dose of about 1 ng, about 2 ng, about 3 ng, about 4 ng, about 5 ng, about 6 ng, about 7 ng, about 8 ng, about 9 ng, about 10 ng, about 12 ng, about 15 ng, about 18 ng, about 20 ng, about 30 ng, about 40 ng, about 50 ng, about 60 ng, about 70 ng, about 80 ng, about 90 ng, about 100 ng, about 120 ng, about 150 ng, about 180 ng, about 200 ng, about 300 ng, about 400 ng, about 500 ng of the modified FGF-1 polypeptide. In some embodiments, the administering comprises administering a dose of about 1 ng to 200 ng, about 3 ng to 180 ng, about 5 ng to 150 ng, about 10 ng to 120 ng, about 20 ng to 100 ng, about 30 ng to 90 ng, about 40 ng to 70 ng, or about 50 ng to 200 ng of the modified FGF-1 polypeptide.

In some embodiments, the administering comprises administering a first dose and a second dose, wherein the second dose is equal to the first dose. In some embodiments, the first dose or second dose comprises a dose disclosed herein. In some embodiments, the first or second dose is at least about 1 ng, 3 ng, 5 ng, 10 ng, 30 ng, 50 ng, 100 ng, 150 ng, or 200 ng of the modified FGF-1 polypeptide. In some embodiments, the administering comprises administering a first dose and a second dose, wherein the second dose is lower than the first dose. In some embodiments, the first dose or second dose comprises a dose disclosed herein. In some embodiments. In some embodiments, the first or second dose is at least about 1 ng, 3 ng, 5 ng, 10 ng, 30 ng, 50 ng, 100 ng, 150 ng, or 200 ng of the modified FGF-1 polypeptide. In some embodiments, the administering comprises administering a first dose and a second dose, wherein the second dose is higher than the first dose. In some embodiments, the first dose or second dose comprises a dose disclosed herein. In some embodiments. In some embodiments, the first or second dose is at least about 1 ng, 3 ng, 5 ng, 10 ng, 30 ng, 50 ng, 100 ng, 150 ng, or 200 ng of the modified FGF-1 polypeptide.

In some embodiments, the administering comprises administering a first dose, a second dose, and a third dose, wherein the second dose is equal to or lower than the first dose, and the third dose is equal to or lower than the second dose. In some embodiments, the first, second or third dose comprises a dose disclosed herein. In some embodiments, the first, second, or third dose is at least about 1 ng, 3 ng, 5 ng, 10 ng, 30 ng, 50 ng, 100 ng, 150 ng, or 200 ng of the modified FGF-1 polypeptide.

In some embodiments, the administering comprises administering a first dose, a second dose, and a third dose, wherein the second dose is equal to or higher than the first dose, and the third dose is equal to or higher than the second dose. In some embodiments, the first, second or third dose comprises a dose disclosed herein. In some embodiments, the first, second, or third dose is at least about 1 ng, 3 ng, 5 ng, 10 ng, 30 ng, 50 ng, 100 ng, 150 ng, or 200 ng of the modified FGF-1 polypeptide.

In some embodiments, the administering comprises administering a first dose, a second dose, a third dose, and a fourth dose. In some embodiments, the first, second, third or fourth dose comprises a dose disclosed herein. In some embodiments, the first, second, third, or fourth dose is at least about 1 ng, 3 ng, 5 ng, 10 ng, 30 ng, 50 ng, 100 ng, 150 ng, or 200 ng of the modified FGF-1 polypeptide. In some embodiments, the administering comprises administering a first dose, a second dose, a third dose, a fourth dose, and a fifth dose, wherein the first, second, third, fourth, or fifth dose comprises a dose disclosed herein, or is at least about 1 ng, 3 ng, 5 ng, 10 ng, 30 ng, 50 ng, 100 ng, 150 ng, or 200 ng of the modified FGF-1 polypeptide. In some embodiments, the administering comprises administering at least five doses, wherein each of the at least five doses is at least about 1 ng, 3 ng, 5 ng, 10 ng, 30 ng, 50 ng, 100 ng, 150 ng, or 200 ng of the modified FGF-1 polypeptide.

In some embodiments, the administering comprises administering a dose of the modified FGF-1 polypeptide before the procedure of DWEK or DSO. In some embodiments, the administering comprises administering a dose of the modified FGF-1 polypeptide concurrently with the procedure of DWEK or DSO. In some embodiments, the administering comprises administering a dose of the modified FGF-1 polypeptide after the procedure of DWEK or DSO.

In some embodiments, the administering comprises administering a dose of the modified FGF-1 polypeptide at least 1 day, 2 days, 3 days, 4 days, 5 days, 6 days, 7 days, 2 weeks, 3 weeks, or 4 weeks before the procedure of DWEK or DSO. In some embodiments, the administering comprises administering a dose of the modified FGF-1 polypeptide at most 1 day, 2 days, 3 days, 4 days, 5 days, 6 days, 7 days, 2 weeks, 3 weeks, or 4 weeks before the procedure of DWEK or DSO. In some embodiments, the administering comprises administering a dose of the modified FGF-1 polypeptide 2-3 days before the procedure of DWEK or DSO. In some embodiments, the administering comprises administering a dose of the modified FGF-1 polypeptide on the same day of the procedure of DWEK or DSO. In some embodiments, the administering comprises administering a dose of the modified FGF-1 polypeptide at least 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the administering comprises administering a dose of the modified FGF-1 polypeptide at most 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO.

In some embodiments, the administering comprises administering a dose of the modified FGF-1 polypeptide on a weekly basis at least 1 week, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the administering comprises administering a dose of the modified FGF-1 polypeptide on a weekly basis at most 1 week, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the administering comprises administering a dose of the modified FGF-1 polypeptide on a weekly basis 3 weeks after the procedure of DWEK or DSO. In some embodiments, the administering comprises administering a dose of the modified FGF-1 polypeptide on the 7th, 14th, 21st day after the procedure of DWEK or DSO.

In some embodiments, the administering comprises administering at least about 10 microliter (mcL), 20 mcL, 30 mcL, 40 mcL, 50 mcL, 60 mcL, 70 mcL, 80 mcL, 90 mcL, or 100 mcL of the pharmaceutical composition. In some embodiments, the pharmaceutical composition has a concentration of about 1 ng/mcL of the modified FGF-1 polypeptide. In some embodiments, the administering comprises administering at most about 10 microliter (mcL), 20 mcL, 30 mcL, 40 mcL, 50 mcL, 60 mcL, 70 mcL, 80 mcL, 90 mcL, or 100 mcL of the pharmaceutical composition. In some embodiments, the pharmaceutical composition has a concentration of about 1 ng/mcL of the modified FGF-1 polypeptide. In some embodiments, the administering comprises administering about 10 microliter (mcL), 20 mcL, 30 mcL, 40 mcL, 50 mcL, 60 mcL, 70 mcL, 80 mcL, 90 mcL, or 100 mcL of the pharmaceutical composition having a concentration of 1 ng/mcL of the modified FGF-1 polypeptide.

In some embodiments, the administering comprises administering one to three times a day. In some embodiments, the administering comprises administering once a day. In some embodiments, the administering comprises administering two times a day. In some embodiments, the administering comprises administering three times a day. In some embodiments, the administering comprises administering one to three times a week. In some embodiments, the administering comprises administering once a week. In some embodiments, the administering comprises administering two times a week. In some embodiments, the administering comprises administering three times a week.

In some embodiments, the pharmaceutical composition is administered by microneedle.

In some embodiments, the administering comprises administering to one eye. In some embodiments, the administering comprises administering to both eyes. In some embodiments, the DWEK or DSO is performed in one eye. In some embodiments, the DWEK or DSO is performed in both eyes. In some embodiments, the cataract surgery is performed in one eye. In some embodiments, the cataract surgery is performed in both eyes. In some embodiments, cataract surgery and DWEK or DSO are performed at the same time (in the same surgical session), in one or both eyes.

In some embodiments, the administering results in a therapeutic effect comprising one or more selected from the group comprising a reduction in corneal endothelial damage, an increase in migration of corneal endothelial cell, an increase in proliferation of corneal endothelial cell, a reduction in corneal edema, an improvement in Best-Corrected Visual Acuity (BCVA), a decrease in corneal thickness, a decrease in central corneal thickness, an improvement in the measurement of visual function and corneal health status (V-FUCHS), a reduction in intraocular pressure, an improvement of slit lamp examination, an improvement in central corneal endothelial cell count, an improvement in peripheral corneal endothelial cell count, or a reduction in time of achieving any thereof. In some embodiments, the administering results in a reduction in corneal endothelial damage. In some embodiments, the administering results in an increase in migration of corneal endothelial cell. In some embodiments, the administering results in an increase in proliferation of corneal endothelial cell. In some embodiments, the administering results in a reduction in corneal edema. In some embodiments, the administering results in an improvement in Best-Corrected Visual Acuity (BCVA). In some embodiments, the administering results in a decrease in corneal thickness. In some embodiments, the administering results in a decrease in central corneal thickness. In some embodiments, the administering results in an improvement in the measurement of visual function and corneal health status (V-FUCHS). In some embodiments, the administering results in a reduction in intraocular pressure. In some embodiments, the administering results in an improvement of slit lamp examination. In some embodiments, the administering results in an improvement in central corneal endothelial cell count. In some embodiments, the administering results in an improvement in peripheral corneal endothelial cell count. In some embodiments, the administering results in a reduction in time of achieving any above disclosed thereof.

In some embodiments, the reduction in corneal endothelial damage comprises a reduction in cell death. In some embodiments, the reduction in corneal endothelial damage comprises a reduction in impairment of function. In some embodiments, the reduction in corneal endothelial damage comprises a reduction in cell death and a reduction in impairment of function.

In some embodiments, the administering results in a therapeutic effect at most about 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the administering. In some embodiments, the administering results in a therapeutic effect at least about 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the administering.

In some embodiments, the BCVA is measured using the Early Treatment Diabetic Retinopathy Scale (ETDRS) scoring system that has at least 69 letters and a score of at least 20/40.

In some embodiments, the administering comprises administering to both eyes, and wherein the administering results in a therapeutic effect disclosed herein in both eyes that is similar or same between the two eyes.

In some embodiments, the administering comprises administering to both eyes, wherein the administering results in an improvement in BCVA that is similar or same between the two eyes.

In some embodiments, the administering results in a dose-dependent therapeutic effect. In some embodiments, the therapeutic effect increases with the dose of the modified FGF-1 polypeptide being administered. In some embodiments, the administering results in a therapeutic effect that is not dose-independent.

In some embodiments, the administering results in a therapeutic effect in the subject with or without cataract surgery, wherein the therapeutic effect is similar or same between a subject with cataract surgery and a subject without cataract surgery.

In some embodiments, the administering results in an improvement in corneal edema or central corneal thickness in the subject with or without cataract surgery, wherein the improvement in corneal edema or central corneal thickness is similar or same between a subject with cataract surgery and a subject without cataract surgery. In some embodiments, the administering results in an improvement in BCVA. In some embodiments, the administering results in a reduction or removal of corneal edema. In some embodiments, the administering results in an accelerated reduction or removal of corneal edema.

In some embodiments, the method comprises a treatment course of at least about 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the method comprises a treatment course of at most about 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO. In some embodiments, the method comprises a treatment course of about 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, or 5 years after the procedure of DWEK or DSO.

In some embodiments, the DWEK/DSO comprises a central descemetorhexis of about 4 mm to about 5 mm. In some embodiments, the DWEK/DSO comprises a central descemetorhexis of at least about 2 mm, 3 mm, 4 mm, 5 mm, 6 mm, 7 mm, or 8 mm. In some embodiments, the DWEK/DSO comprises a central descemetorhexis of at most about 2 mm, 3 mm, 4 mm, 5 mm, 6 mm, 7 mm, or 8 mm. In some embodiments, the DWEK/DSO comprises a central descemetorhexis of about 2 mm to 8 mm, 3 mm to 7 mm, 4 mm to 6 mm, or 4 mm to 8 mm.

In some embodiments, a decrease in the DSO size results in a decrease in the period of time to achieve a therapeutic effect in the subject. In some embodiments, the therapeutic effect comprises a faster improvement to BCVA 20/40, an accelerated improvement to BCVA 20/40, a reduced period of time to BCVA 20/40, or a reduction in corneal edema. In some embodiments, the therapeutic effect comprises a faster improvement to BCVA 20/40. In some embodiments, the therapeutic effect comprises an accelerated improvement to BCVA 20/40. In some embodiments, the therapeutic effect comprises a reduced period of time to BCVA 20/40. In some embodiments, the therapeutic effect comprises a reduction in corneal edema.

In some embodiments, the subject has diabetes. In some embodiments, the administering results in a therapeutic effect comprising a reduction in corneal thickness or a reduction in corneal edema. In some embodiments, the corneal disease or condition comprises Fuchs Endothelial Cell Dystrophy (FECD) with central guttae as a contributing cause of visual symptom.

In some embodiments, the visual symptom comprises decreased visual acuity from the cornea, morning blur, or central corneal edema. In some embodiments, the subject has clear peripheral cornea with adequate corneal endothelial cell (CEC) reserve. In some embodiments, the subject does not have sub-epithelial haze or fibrosis. In some embodiments, the subject does not exhibit secondary corneal pathology, refractive surgery in the eye, or extra-ocular inflammation caused by a non-infectious or infectious pathogen in the eye. In some embodiments, the subject is human.

### Pharmaceutical Compositions

Pharmaceutical compositions comprising a modified FGF-polypeptide as described herein may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a modified FGF with other chemical components, such as carriers, stabilizers, diluents, dispersing agents, suspending agents, thickening agents, and/or excipients, and, optionally, other therapeutic and/or prophylactic ingredients. The pharmaceutical composition facilitates administration of the modified FGF to an organism. In practicing the methods of treatment or use provided herein, therapeutically effective amounts of modified FGF-1 polypeptides described herein are administered in a pharmaceutical composition to a mammal having an ocular disease, disorder, or condition to be treated. In some embodiments, the mammal is a human. A therapeutically effective amount can vary widely depending on the severity of the disease, the age and relative health of the subject, the potency of the compound used and other factors. A pharmaceutically acceptable or suitable composition includes an ophthalmologically suitable or acceptable composition.

A pharmaceutical composition (e.g., for delivery by injection or for application as an eye drop) may be in the form of a liquid or solid. A liquid pharmaceutical composition may include, for example, one or more of the following: sterile diluents such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. Physiological saline is commonly used as an excipient, and an injectable pharmaceutical composition or a composition that is delivered ocularly (for example, as an eye drop) is preferably sterile.

A modified FGF-polypeptide or pharmaceutical composition described herein can be delivered to a subject by any suitable means, including, for example, topically, intraocularly, intracamerally, orally, parenterally, intravenously, intraperitoneally, intranasally (or other delivery methods to the mucous membranes, for example, of the nose, throat, and bronchial tubes), or by local administration to the eye, or by an intraocular or periocular device. Modes of local administration can include, for example, topical application, eye drops, intraocular injection or periocular injection. Periocular injection typically involves injection of the compound under the conjunctiva or into the Tennon's space (beneath the fibrous tissue overlying the eye). Intraocular injection typically involves injection of the modified FGF or pharmaceutical composition into the vitreous. In certain embodiments, the administration is non-invasive, such as by topical application or eye drops. In some embodiments, the administration is via a combination of topical and intracameral method.

A modified FGF or pharmaceutical composition described herein can be formulated for administration using pharmaceutically acceptable (suitable) carriers or vehicles as well as techniques routinely used in the art. A pharmaceutically acceptable or suitable carrier includes an ophthalmologically suitable or acceptable carrier. A carrier is selected according to the solubility of the particular modified FGF. Suitable ophthalmological compositions and formulations include those that are administrable locally to the eye, such as by eye drops, injection or the like. In the case of eye drops, the formulation can also optionally include, for example, ophthalmologically compatible agents such as isotonizing agents such as sodium chloride, concentrated glycerin, and the like; buffering agents such as sodium phosphate, sodium acetate, and the like; surfactants such as polyoxyethylene sorbitan mono-oleate (also referred to as Polysorbate 80), polyoxyl stearate 40, polyoxyethylene hydrogenated castor oil, and the like; stabilization agents such as sodium citrate, sodium edentate, and the like; preservatives such as benzalkonium chloride, parabens, and the like; and other ingredients. Preservatives can be employed, for example, at a level of from about 0.001 to about 1.0% weight/volume. The pH of the formulation is usually within the range acceptable to ophthalmologic formulations, such as within the range of about pH 4 to 8.

In some embodiments, the pharmaceutical composition disclosed herein comprises sodium chloride, potassium phosphate monobasic, sodium phosphate di-basic anhydrous, recombinant human albumin solution, or polysorbate 80.

For injection, the modified FGF or pharmaceutical composition can be provided in an injection grade saline solution, in the form of an injectable liposome solution, slow-release polymer system or the like. Intraocular and periocular injections are known to those skilled in the art and are described in numerous publications including, for example, Spaeth, Ed., Ophthalmic Surgery: Principles of Practice, W. B. Sanders Co., Philadelphia, Pa., 85-87, 1990.

In some embodiments, the modified FGF or pharmaceutical composition (e.g., an ophthalmic formulation) is administered via microneedles into the cornea (Jiang et al. (2007). Invest Ophthalmol Vis Sci 48(9): 4038-4043). A microneedle array is coated with the modified FGF or pharmaceutical composition and pressed against the cornea such that the microneedles penetrate into the corneal stroma but do not penetrate the entire cornea. It is then removed, and the modified FGF or pharmaceutical composition is left behind in the corneal stroma. This modified FGF or pharmaceutical composition can stimulate the corneal cells to proliferate and migrate, and suppresses the scarring response that the stromal cells normally have.

For delivery of a composition comprising at least one of the modified FGF-1 polypeptides described herein via a mucosal route, which includes delivery to the nasal passages, throat, and airways, the composition may be delivered in the form of an aerosol. The compound may be in a liquid or powder form for intramucosal delivery. For example, the composition may be delivered via a pressurized aerosol container with a suitable propellant, such as a hydrocarbon propellant (e.g., propane, butane, isobutene). The composition may be delivered via a non-pressurized delivery system such as a nebulizer or atomizer.

Suitable oral dosage forms include, for example, tablets, pills, sachets, or capsules of hard or soft gelatin, methylcellulose or of another suitable material easily dissolved in the digestive tract. Suitable nontoxic solid carriers can be used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

The modified FGF-1 polypeptides or pharmaceutical compositions described herein may be formulated for sustained or slow-release. Such compositions may generally be prepared using well known technology and administered by, for example, periocular, intraocular, rectal, oral or subcutaneous implantation, or by implantation at the desired target site, or by topical application. Sustained-release formulations may contain an agent dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active compound contained within a sustained-release formulation depends upon the site of implantation, the rate and expected duration of release, and the nature of the condition to be treated or prevented.

Systemic drug absorption of a drug or composition administered via an ocular route is known to those skilled in the art (see, e.g., Lee et al., Int. J Pharm. 233:1-18 (2002)). In one embodiment, a compound described herein is delivered by a topical ocular delivery method (see, e.g., Curr. Drug Metab. 4:213-22 (2003)). The composition may be in the form of an eye drop, salve, or ointment or the like, such as, aqueous eye drops, aqueous ophthalmic suspensions, non-aqueous eye drops, and non-aqueous ophthalmic suspensions, gels, ophthalmic ointments, etc. For preparing a gel, for example, carboxyvinyl polymer, methyl cellulose, sodium alginate, hydroxypropyl cellulose, ethylene maleic anhydride polymer and the like can be used.

In another embodiment, the modified FGF solution or pharmaceutical composition (e.g., an ophthalmic formulation) contains hyaluronic acid, carboxymethyl cellulose, or other polysaccharides that provide increased ocular tolerability, viscosity and osmolality to produce a comfortable ocular solution.

Pharmaceutical compositions may be administered in a manner appropriate to the disease, disorder, or condition to be treated (or prevented) as determined by persons skilled in the medical arts. An appropriate dose and a suitable duration and frequency of administration will be determined by such factors as the condition of the patient, the type and severity of the patient's disease, disorder, or condition, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free, or a lessening of symptom severity). For prophylactic use, a dose should be sufficient to prevent, delay the onset of, or diminish the severity of an ocular disease, disorder, or condition. Optimal doses may generally be determined using experimental models and/or clinical trials. The optimal dose may depend upon the body mass, weight, or blood volume of the patient.

### Kits/Articles of Manufacture

For use in the therapeutic applications described herein, kits and articles of manufacture are also provided herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products include, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of ophthalmic formulations of the modified FGF-1 polypeptides and pharmaceutical compositions provided herein are contemplated as are a variety of treatments for any ocular disease, disorder, or condition that would benefit by administration of a modified FGF ore pharmaceutical composition described herein.

For example, the container(s) can include a modified FGF such as a modified FGF-1 having a sequence of SEQ ID NO: 2. The container(s) optionally have a sterile access port. Such kits optionally comprising compounds with an identifying descriptions or labels or instructions relating to their use in the methods described herein.

A kit typically may include one or more additional containers, each with one or more of various materials (such as reagents, optionally in concentrated form, and/or devices) desirable from a commercial and user standpoint for use of a modified FGF described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, a modified FGF pharmaceutical composition can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a modified FGF provided herein formulated in a compatible pharmaceutical carrier can also be prepared, placed in an appropriate container, and labeled for treatment of an indicated condition.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “Percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Alignment for purposes of determining percent amino acid sequence identity can for example be achieved using publicly available sequence comparison computer program ALIGN-2. The source code for the ALIGN-2 sequence comparison computer program is available with user documentation in the U.S. Copyright Office, Washington D.C., 20559, where it is registered under U.S. Copyright Registration No. TXU510087. The ALIGN-2 program can be compiled for use on a UNIX operating system, such as a digital UNIX V4.0D. All sequence comparison parameters are set by the ALIGN-2 program and do not vary.

Definition of standard chemistry terms may be found in reference works, including but not limited to, Carey and Sundberg “ADVANCED ORGANIC CHEMISTRY 4TH ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

The terms “treat,” “treating” or “treatment” include alleviating, abating or ameliorating a disease, disorder or condition symptoms, preventing additional symptoms, ameliorating or preventing the underlying metabolic causes of symptoms, inhibiting the disease, disorder, or condition, e.g., arresting the development of the disease, disorder or condition, relieving the disease, disorder or condition, causing regression of the disease, disorder or condition, relieving a condition caused by the disease, disorder or condition, or stopping the symptoms of the disease, disorder or condition. The terms “treat,” “treating” or “treatment”, include, but are not limited to, prophylactic and/or therapeutic treatments.

The term “acceptable” or “pharmaceutically acceptable”, with respect to a formulation, composition or ingredient, refers to having no persistent detrimental effect on the general health of the subject being treated or does not abrogate the biological activity or properties of the modified FGF described herein, and is relatively nontoxic.

The term “amelioration” of the symptoms of a particular disease, disorder or condition by administration of a particular modified FGF or pharmaceutical composition refers to any lessening of severity, delay in onset, slowing of progression, or shortening of duration, whether permanent or temporary, lasting or transient that can be attributed to or associated with administration of the modified FGF or pharmaceutical composition.

The term “combination” or “pharmaceutical combination” as used herein, means a product that results from the mixing or combining of more than one active ingredient and includes both fixed and non-fixed combinations of the active ingredients. The term “fixed combination” means that one active ingredient (e.g., a modified FGF) and a co-agent are both administered to a patient simultaneously in the form of a single entity or dosage. The term “non-fixed combination” means that one active ingredient (e.g., a modified FGF) and a co-agent are administered to a patient as separate entities either simultaneously, concurrently or sequentially with no specific intervening time limits, wherein such administration provides effective levels of the two agents in the body of the patient. The latter also applies to cocktail therapy, e.g., the administration of three or more active ingredients.

The term “pharmaceutical composition” as used herein refers to one or more modified FGF-1 polypeptides with one or more other chemical components, such as carriers, stabilizers, diluents, dispersing agents, suspending agents, thickening agents, and/or excipients. The pharmaceutical composition facilitates administration of the modified FGF-1 polypeptides to an organism. Multiple techniques of administering a modified FGF-1 polypeptide exist in the art including, but not limited to: topical, ophthalmic, intraocular, periocular, intravenous, oral, aerosol, parenteral, and administration.

The term “carrier,” as used herein, refers to relatively nontoxic chemical compounds or agents that facilitate the incorporation of an agent of interest (e.g., a modified FGF) into cells or tissues.

The term “diluent” refers to chemical compounds that are used to dilute the agent of interest (e.g., a modified FGF) prior to delivery. Diluents can also be used to stabilize agents because they can provide a more stable environment. Salts dissolved in buffered solutions (which also can provide pH control or maintenance) are utilized as diluents in the art, including, but not limited to a phosphate buffered saline solution.

The terms “co-administration” or the like, are meant to encompass administration of the selected agents (e.g., a modified FGF or composition thereof and a co-agent) to a single patient, and are intended to include treatment regimens in which the agents are administered by the same or different route of administration or at the same or different time.

The terms “effective amount” or “therapeutically effective amount,” refer to a sufficient amount of a modified FGF-1 polypeptide, agent, combination or pharmaceutical composition described herein administered which will relieve to some extent one or more of the symptoms of the disease, disorder or condition being treated. The result can be reduction and/or alleviation of the signs, symptoms, or causes of a disease, or any other desired alteration of a biological system. For example, an “effective amount” for therapeutic uses is the amount of the modified FGF, agent, combination or pharmaceutical composition required to provide a desired pharmacologic effect, therapeutic improvement, or clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. It is understood that “an effect amount” can vary from subject to subject due to variation in metabolism of the modified FGF, combination, or pharmaceutical composition, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The term “prophylactically effective amount,” refers that amount of a modified FGF, compound, agent, combination or pharmaceutical composition described herein applied to a patient which will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “subject” or “patient” as used herein, refers to an animal, which is the object of treatment, observation or experiment. By way of example only, a subject may be, but is not limited to, a mammal including, but not limited to, a human.

The terms “enhance” or “enhancing” means to increase or prolong either in potency or duration a desired effect. By way of example, “enhancing” the effect of therapeutic agents singly or in combination refers to the ability to increase or prolong, either in potency, duration and/or magnitude, the effect of the agents on the treatment of a disease, disorder or condition. When used in a patient, amounts effective for this use will depend on the severity and course of the disease, disorder or condition, previous therapy, the patient's health status and response to the drugs, and the judgment of the treating physician.

The term “modulate,” means to interact with a target (e.g., a FGF receptor) either directly or indirectly so as to alter the activity of the target, including, by way of example only, to enhance the activity of the target, to inhibit or antagonize the activity of the target, to limit the activity of the target, or to extend the activity of the target. In some embodiments, modified FGF-1 polypeptides and pharmaceutical compositions described herein can modulate the activity of one or more respective targets (e.g., one or more FGF receptors). In some embodiments, the modified FGF-1 polypeptides described herein modulate (e.g., increase) the activity of one or more FGF receptors on a cell (e.g., a corneal endothelial cell), resulting, e.g., in cell migration and/or cell proliferation.

As used herein, the term “target” or refers to a biological molecule (e.g., a target protein or protein complex), such as an FGF receptor, or a portion of a biological molecule capable of being bound by a selective binding agent (e.g., a modified FGF) or pharmaceutical composition described herein. As used herein, the term “non-target” refers to a biological molecule or a portion of a biological molecule that is not selectively bound by a selective binding agent or pharmaceutical composition described herein.

The term “target activity” or “cell response” refers to a biological activity capable of being modulated by a modified FGF or any cellular response that results from the binding of a modified FGF to a FGF receptor. Certain exemplary target activities and cell responses include, but are not limited to, binding affinity, signal transduction, gene expression, cell migration, cell proliferation, cell differentiation, and amelioration of one or more symptoms associated with an ocular disease, disorder or condition.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The expressions “at least one of A and B” and “at least one of A or B” may be construed to mean at least A, at least B, or at least A and B (i.e., a set comprising A and B, which set may include one or more additional elements). The term “A and/or B” may be construed to mean only A, only B, or both A and B.

The expressions “at least about A, B, and C” and “at least about A, B, or C” may be construed to mean at least about A, at least about B, or at least about C. The expressions “at most about A, B, and C” and “at most about A, B, or C” may be construed to mean at most about A, at most about B, or at most about C.

The expression “between about A and B, C and D, and E and F” may be construed to mean between about A and about B, between about C and about D, and between about E and about F. The expression “between about A and B, C and D, or E and F” may be construed to mean between about A and about B, between about C and about D, or between about E and about F.

The expression “about A to B and C to D” may be construed to mean between about A and about B and between about C and about D. The expression “about A to B or C to D” may be construed to mean between about A and about B or between about C and about D.

The term “exemplary” as used herein means “serving as an example, instance, or illustration.” Any embodiment described herein as “exemplary” is not to be construed as preferred or advantageous over other embodiments.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

### EXAMPLES

The following examples are included for illustrative purposes only and are not intended to limit the scope of the invention.

### Example 1: Clinical Effects of a Modified FGF-1 Polypeptide (TTHX114)

### Non-Clinical Studies

The nonclinical program for TTHX1114 had several objectives: (1) understand in vitro, ex vivo and in vivo pharmacodynamics of TTHX1114; (2) evaluate ocular pharmacokinetics (PK) following a single Intracameral (IC) injection in rabbits; and (3) evaluate ocular and systemic safety, toxicokinetics (TK) and immunogenicity of TTHX1114 following repeat administrations up to 1-month by weekly IC injections in rabbits and dogs and by intravenous (IV) injections every other day in rats.

Safety pharmacology parameters evaluated in toxicity studies demonstrated that TTHX1114 caused no adverse effects on central nervous system function in a 1-month IV toxicity study in rats and no effects on respiratory and cardiovascular system function in a 1-month IC toxicity study in dogs.

TK evaluation in the pivotal 1-month IV toxicity study in rats with administration of ≤50 μg/kg TTHX1114 every other day demonstrated systemic exposures (Cₘₐₓ and AUCₗₐₛₜ) that increased in a dose proportional manner with no gender differences and 2- to 4-fold accumulation with repeated dosing.

The lack of systemic effects and systemic exposure in 1-month IC toxicity studies in animals at ocular doses that are 880- and 88-times the planned starting and highest ocular doses, respectively, in the Phase 1/Phase 2 clinical trial, combined with the facts that the LLOQ to be used for human PK is 7.6-times lower than LOEL systemic exposure in rats and 17-times lower than the ED₅₀ potency value in NIH 3T3 cell proliferation assay, indicated that there are high ocular safety margins and systemic exposure will unlikely be detected and that the LLOQ represents negligible systemic exposure in humans.

The nonclinical program for TTHX1114 supports the repeat administration of TTHX114 up to a total of 5 injections.

### Potential Risks and Benefits

Anticipated hypothetical adverse events included, but were not limited to: anterior chamber flare, conjunctival inflammation, endophthalmitis, and inflammation of the anterior chamber (e.g.: cornea and/or iris).

Complications of DWEK/DSO were: descemetorhexis decentration, descemet's membrane detachment, posterior stromal opacities, abnormal corneal topography, and persistent corneal edema

### Rationale for Dose Selection

Dose levels of up to 1000 ng have been evaluated in nonclinical studies and the highest dose planned in this study was well below the highest non-clinical dose level evaluated.

A prior study of TTHX-001 was designed to determine the Recommended Phase 2 Dose for TTHX1114 and evaluated dose levels of 1 ng, 3 ng, and 10 ng. The maximum tolerated dose (MTD) was not exceeded. All doses in Study TTHX-001 were administered via a 10 microliter (mcL) IC injection.

The normal adult human eye contains approximately 300 mcL of aqueous humor in the Anterior Chamber. As TTHX1114 was provided at a concentration of 1000 ng/mL an increase in dose required an increase in volume. Turnover of the aqueous humor (AqHu) in the anterior chamber in the eye was approximately 90 minutes, during which any significant increase in IOP was normalized via drainage through the trabecular meshwork. Patients with uncontrolled glaucoma were excluded from this study.

### Dose Limiting Toxicity

Potential dose limiting toxicity (DLT) is defined as a suspected adverse reaction of: (1) Post-injection IOP measurement of >20% from pre-injection lasting more than 90 minutes; and (2) IOP that remains higher than 32 mmHg and not responding to therapy.

If any subject experienced a potential DLT, additional study treatment was held, and the Medical Monitor was contacted to discuss medical management of the subject including possible treatment and additional unscheduled study assessments.

10 ng (10 mcL volume): Study TTHX-001 evaluated the safety and tolerability of TTHX1114 dose levels up to 10 ng administered weekly to nonsurgical eyes. The 10 mcL volume IC injection appeared to have been well tolerated and there were no clinically significant changes in IOP, or any IOP-related AEs reported.

30 ng (30 mcL volume): The 10 mcL volume IC injection appeared to have been well tolerated; a 30 mcL volume injection represented an approximate potential 10% increase fluid volume in the anterior chamber. The 30 mcL volume injection was used as a “step-up” in all subjects. Following demonstrated tolerability (No DLT) of the 30 mcL volume injection, subjects received 50 ng/50 mcL doses of TTHX1114. After at least 10 IC injection of 30 mcL have been safely tolerated, the “step-up” dose was removed, and subjects received 50 ng/50 mcL on non-surgical days.

50 ng (50 mcL volume): A 50 mcL volume injection represented an approximate potential 17% increase fluid volume in the anterior chamber. Intrasubject dose escalation occurred after subjects have demonstrated tolerability (No DLT) of the 30 mcL volume injection. After at least 10 IC injection of 30 mcL have been safely tolerated, the “step-up” dose was removed and subjects received 50 ng/50 mcL on non-surgical days

100 ng (100 mcL volume): During DWEK/DSO, the Anterior Chamber was surgically opened with resultant loss of some aqueous humor; the dose volume of 100 mcL did not restore the entire preoperative volume leaving the post-operative eye in the desired semi-deflated state. As described above, there were no concerns identified with potential increases in IOP following a 50-mcL volume administration and 100 mcL volume injections of surgical days.

### Study Population

The study population consisted of men and women aged ≥18 years with a confirmed diagnosis of FECD who will undergo DWEK/DSO.

### Trial Design

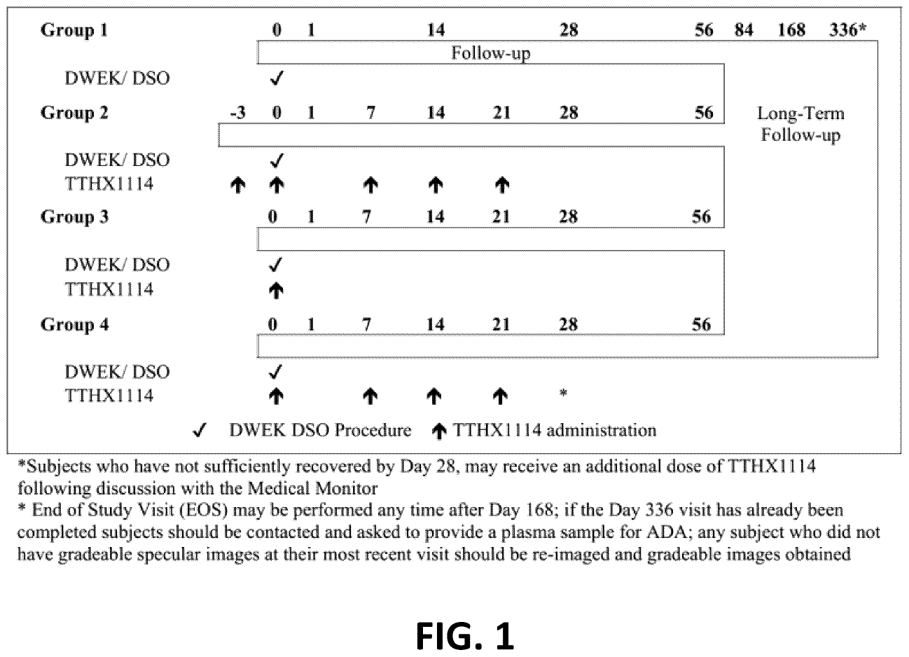

This was an open-label, multi-center study in which up to approximately 50 subjects with FECD who were scheduled for DWEK/DSO was enrolled. Study subjects were assigned to Study Treatment Group based on the Group(s) open at the time of treatment of the primary (first) eye; if a subject elects to have an eligible second (contralateral) eye treated, that “fellow” eye was treated based on the Group(s) open at the time. The number of subjects per group in the study and per study treatment is summarized below in Table 1 and FIG. 1.

The study allowed treatment of the contralateral eye. To avoid ambiguity the eye that was treated first will be referred to as the Study Eye (SE); the contralateral eye will be referred to as the Fellow Eye (FE).

TABLE 1
Study Groups and Number of Subjects

| | TTHX1114 Regimen | | |
|---|---|---|---|
| | Day | Dose | Number of Subjects |
| Group 1 | N/A | N/A | 8 |
| | | | (Closed after 1 enrolled) |
| Group 2 | Day −3 | 10 ng | 8-16 |
| | Day 0 | 50 ng | (Closed after 9 enrolled) |
| | Day 7 | 10 ng | |
| | Day 14 | 10 ng | |
| | Day 21 | 10 ng | |
| Group 3 | Day 0 | 100 ng | 20 |
| Group 4 | Day 0 | 100 ng | 20 |
| | Day 7 | 30 or 50 ng | |
| | Day 14 | 30 or 50 ng | |
| | Day 21 | 30 or 50 ng | |
| | | | up to 50 |

Subject was consented and screened or eligibility during the screening period prior to the scheduled DWEK/DSO procedure.

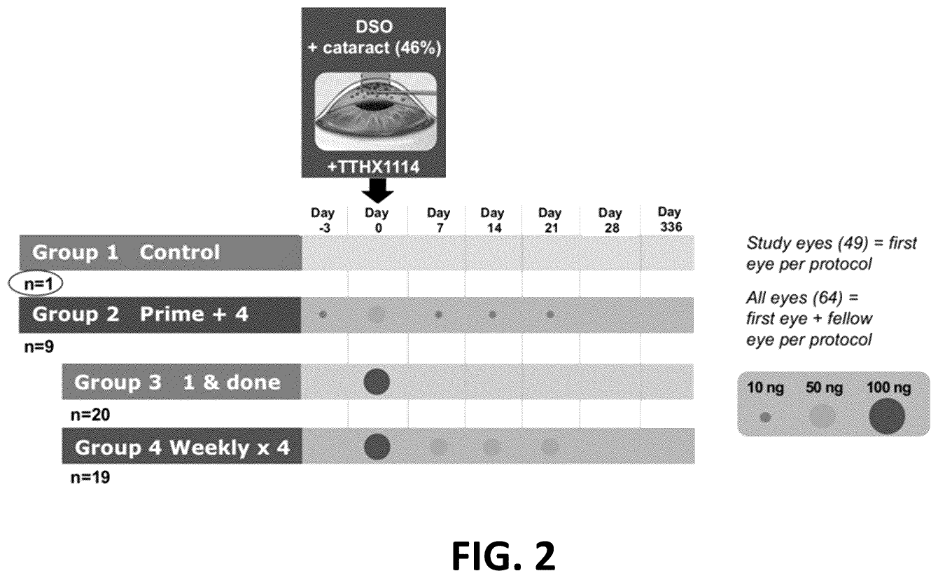

The study schema is presented in FIG. 1 and FIG. 2.

Eligible subjects who did not wish to receive investigational product or who did not have a qualifying Fellow Eye were assigned to Treatment Group 1.

Following preliminary safety and tolerability assessment of the 50 ng IC injection in Group 2, it was elected to open Group 3 and Group 4.

Group 3 in which eligible subjects was given a “1 and done” dosing regimen in which TTHX1114 was administered as a 100 ng/100 mcL volume injection at the time of DWEK/DSO.

Group 4 in which eligible subjects was given a 100 ng/100 mcL volume injection at the time of DWEK/DSO and then weekly injections of 30 or 50 ng (30 or 50 mcL)×3. Subjects in Group 4, who have not sufficiently recovered by Day 28, received an additional dose of TTHX1114 following discussion with the Medical Monitor.

Study Treatment Group 1: Study subjects in Group 1 underwent Descemetorhexis without Endothelial Keratoplasty (DWEK/DSO) on Study Day 0 and did not receive any TTHX1114. Subjects in this group functioned as a control arm and following DWEK/DSO was seen in follow-up on: Day 1, Day 14, Day 28, and Day 56. Assessments conducted at follow-up visits included routine ocular assessments.

TABLE 2
Schedule of Events Group 1
Group 1

| | Screening | | | 14 | 28 | 56 | 84 | 168 | 336 |
|---|---|---|---|---|---|---|---|---|---|
| | (≈28 days) | 0* | 1 | (±1) | (±3) | (±3) | (±21) | (±21) | (±21) |
| Informed Consent | X | | | | | | | | |
| Demographics | X | | | | | | | | |
| Medical and Ocular History | X | | | | | | | | |
| Comprehensive Ocular Examination | SE | | | | SE | SE | | | |
| (including slit lamp examination | | | | | | | | | |
| and dilated exam of the lens, | | | | | | | | | |
| fundus, and vitreous) | | | | | | | | | |
| Slit Lamp Biomicroscopy | SE^{a} | SE | SE | SE | SE^{a} | SE^{a} | | | |
| Specular Microscopy | SE | | | SE | SE | SE | | | SE |
| Assessment of Corneal Thickness^{b} | SE | SE | | SE | SE | SE | SE | SE | SE |
| Best-corrected Visual Acuity | SE | SE | SE | SE | SE | SE | SE | SE | SE |
| Intraocular Pressure (IOP) Measurement | SE | SE | SE | SE | SE | SE | | | |
| Optional Blood for Genetic Testing | | X | | | | | | | |
| V-FUCHS | X | | | | X | X | | | |
| DWEK/DSO | | X | | | | | | | |
| Collect Endothelium Tissue sample | | X | | | | | | | |
| Collect Aqueous Humor sample | | X | | | | | | | |
| Concomitant Medications Review | X | X | X | X | X | X | | | |
| Assess Adverse Events | | X | X | X | X | X | | | |

OU = both eyes,
SE = Study Eye,
V-FUCHS = Visual Function and Corneal Health Status
Note:
Unless otherwise specified assessments to be performed prior to study treatment injection, if applicable
*Day 0 pre-treatment ocular assessments may be obtained within the 7 days prior to Day 0 (if screening visit assessments were within 7 days, they do not need to be repeated)
^{a}as part of Comprehensive Eye Examination
^{b}measured by AS-OCT (preferred) if AS-OCT not available, Pentacam or Ultrasound Pachymetry is to be used
(Note:
subjects must be assessed using the same modality throughout the study)

Study Treatment Group 2: Approximately 48 to 72 hours prior to DWEK/DSO (Day −3), study subjects received a “conditioning dose” (10 ng [10 mcL]) of TTHX1114. The conditioning dose was intended to provide anti-apoptotic protection for the existing cells. On Day 0, subjects underwent DWEK/DSO and received TTHX1114 (50 ng [50 mcL]) at the time of the surgical procedure. As described above, the immediate post-surgical anterior chamber should easily accommodate the additional volume. Study subjects returned for follow-up on Day 1 (the day after DWEK/DSO) and subsequent study treatment administration on: Day 7, Day 14, and Day 21. Follow-up visits occurred on: Day 28 and Day 56.

TABLE 3
Schedule of Events Group 2
Group 2

| | Screening | Study Day | | | | | | | | | | |
|---|---|---|---|---|---|---|---|---|---|---|---|---|
| | (≈28 | | | | 7 | 14 | 21 | 28 | 56 | 84 | 168 | 336* |
| | days) | −3 | 0 | 1 | (±1) | (±1) | (±1) | (±3) | (±3) | (±21) | (±21) | (±21) |
| Informed Consent | X | | | | | | | | | | | |
| Demographics | X | | | | | | | | | | | |
| Medical and Ocular History | X | | | | | | | | | | | |
| Comprehensive Ocular | SE | | | | | | | SE | SE | | | |
| Examination | | | | | | | | | | | | |
| Slit Lamp | SE^{a} | SE | SE | SE | SE | SE | SE | SE^{a} | SE^{a} | | | |
| Specular Microscopy | SE | | | | | SE | SE | SE | SE | SE | | SE |
| Assessment of Corneal | | SE | | | | SE | SE | SE | SE | SE | SE | SE |
| Thickness^{b} | | | | | | | | | | | | |
| Best-corrected Visual | SE | SE | | SE | SE | SE | SE | SE | SE | SE | SE | SE |
| Acuity | | | | | | | | | | | | |
| Intraocular Pressure (IOP) | SE | | | | | | | SE | SE | | | |
| measurement | | | | | | | | | | | | |
| Pre-injection/procedure | | SE | SE | | SE | SE | SE | | | | | |
| Post-injection | | SE | SE | | SE | SE | SE | | | | | |
| Urine pregnancy test | X | | | | | | | X | | | | |
| (WOCBP) | | | | | | | | | | | | |
| Clinical Laboratory | | X | | | | | | X | | | | |
| Collection | | | | | | | | | | | | |
| Plasma for ADA | | | | | | | | X | | | | X |
| Pre-injection | | X | | | | | | | | | | |
| Plasma for | | | | | | | | X | | | | |
| Pharmacokinetics (PK) | | | | | | | | | | | | |
| Pre-injection | | X | X | | | | | | | | | |
| 1.5 hours after injection | | | X | | | | | | | | | |
| Optional Blood for Genetic | | X | | | | | | | | | | |
| Testing | | | | | | | | | | | | |
| V-FUCHS | X | | | | | | | X | X | | | |
| DWEK/DSO | | | X | | | | | | | | | |
| Collect endothelium | | | X | | | | | | | | | |
| tissue sample | | | | | | | | | | | | |
| Collect Aqueous | | | X | | | | | | | | | |
| Humor sample | | | | | | | | | | | | |
| Study Treatment IC | | X | X | | X | X | X | | | | | |
| Injection (SE) | | | | | | | | | | | | |
| Concomitant Medications | X | X | X | X | X | X | X | X | X | | | |
| Review | | | | | | | | | | | | |
| Assess Adverse Events | | X | X | X | X | X | X | X | X | | | |

OU = both eyes,
SE = Study Eye,
V-FUCHS = Visual Function and Corneal Health Status
Note:
Unless otherwise specified assessments to be performed prior to study treatment injection, if applicable
*Day −3 pre-treatment ocular assessments may be obtained within the 7 days prior to Day 0 (if screening visit assessments were within 7 days, they do not need to be repeated)
^{a}as part of Comprehensive Eye Examination
^{b}measured by AS-OCT (preferred) if AS-OCT not available, Pentacam, or Ultrasound Pachymetry
is to be used
(Note:
subjects must be assessed using the same modality throughout the study)
^{c} IOP pre-injection and then post-injection every 30 to 60 minutes until less than at least 5 mmHg higher than pre-injection IOP
(Note:
IOP may not be measurable post injection on Day 0)
*End of Study Visit (EOS) may be performed any time after Day 168; if the Day 336 visit, if the subject has already completed the study, the subject should be contacted and asked to provide a plasma sample for ADA; any subject who did not have gradeable specular images at their most recent visit should be re-imaged and gradeable images obtained

Study Treatment Group 3: On Day 0, subjects underwent DWEK/DSO and received TTHX1114 (100 ng [100 mcL]) at the time of the surgical procedure. As described above, the immediate post-surgical anterior chamber should easily accommodate the additional volume. Study subjects returned for follow-up on Day 1 (the day after DSO) and subsequent follow-up visits occurred on: Day 7, Day 14, Day 21, Day 28, and Day 56.

TABLE 4
Schedule of Events Group 3
Study TTHX-002: Group 3

| | Screening | Study Day | | | | | | | | | |
|---|---|---|---|---|---|---|---|---|---|---|---|
| | (≈28 | | | 7 | 14 | 21 | 28 | 56 | 84 | 168 | 336* |
| | days) | 0* | 1 | (±1) | (±1) | (±1) | (±3) | (±3) | (±21) | (±21) | (±21) |
| Informed Consent | X | | | | | | | | | | |
| Demographics | X | | | | | | | | | | |
| Medical and Ocular History | X | | | | | | | | | | |
| Comprehensive Ocular | SE | | | | | | SE | SE | | | SE |
| Examination (including slit | | | | | | | | | | | |
| lamp examination and | | | | | | | | | | | |
| dilated exam of the lens, | | | | | | | | | | | |
| fundus, vitreous and | | | | | | | | | | | |
| MAC-OCT) | | | | | | | | | | | |
| Slit Lamp | SE^{a} | SE | SE | SE | SE | SE | SE^{a} | SE^{a} | | | SE |
| Specular Microscopy (incl | SE | | | | | | SE | SE | SE | | SE |
| peripheral images at | | | | | | | | | | | |
| Screening) | | | | | | | | | | | |
| Assessment of Corneal | | SE | | SE | SE | SE | SE | SE | SE | SE | SE |
| Thickness^{b} | | | | | | | | | | | |
| Pentacam | | SE | | | | | SE | SE | | | |
| Best-corrected Visual Acuity | SE | SE | | | SE | SE | SE | SE | SE | SE | SE |
| Intraocular Pressure (IOP) | SE | | SE | SE | | | | SE | | | |
| measurement | | | | | | | | | | | |
| Pre-injection/procedure | | SE | | | | | | | | | |
| Post-injection | | SE | | | | | | | | | |
| Urine pregnancy test | X | | | | | | X | | | | |
| (WOCBP) | | | | | | | | | | | |
| Plasma for ADA | | X | | | | | X | | | | X |
| Pre-injection | | | | | | | | | | | |
| Plasma for | | | | | | | X | | | | |
| Pharmacokinetics (PK) | | | | | | | | | | | |
| Pre-injection | | X | | | | | | | | | |
| 1.5 hours after injection | | X | | | | | | | | | |
| Optional Blood for | | X | | | | | | | | | |
| Genetic Testing | | | | | | | | | | | |
| V-FUCHS | X | | | | | | X | X | | | |
| DWEK/DSO | | X | | | | | | | | | |
| Collect endothelium | | X | | | | | | | | | |
| tissue sample | | | | | | | | | | | |
| Collect Aqueous Humor | | X | | | | | | | | | |
| sample | | | | | | | | | | | |
| Study Treatment IC | | 100 | | | | | | | | | |
| Injection (SE) | | ng | | | | | | | | | |
| Concomitant Medications | X | X | X | X | X | X | X | X | | | |
| Review | | | | | | | | | | | |
| Assess Adverse Events | | X | X | X | X | X | X | X | | | |

OU = both eyes,
SE = Study Eye,
V-FUCHS = Visual Function and Corneal Health Status
Note:
Unless otherwise specified assessments to be performed prior to study treatment injection, if applicable
*Day 0 pre-treatment ocular assessments may be obtained within the 7 days prior to Day 0 (if screening visit assessments were within 7 days, they do not need to be repeated)
^{a}as part of Comprehensive Eye Examination
^{b}measured by AS-OCT (preferred) if AS-OCT not available, Pentacam, or Ultrasound Pachymetry is to be used
(Note:
subjects must be assessed using the same modality throughout the study)
^{c} IOP pre-injection and then post-injection every 30 to 60 minutes until less than at least 5 mmHg higher than pre-injection IOP
(Note:
IOP may not be measurable post injection on Day 0)
*End of Study Visit (EOS) may be performed any time after Day 168; if the Day 336 visit, if the subject has already completed the study, the subject should be contacted and asked to provide a plasma sample for ADA; any subject who did not have gradeable specular images at their most recent visit should be re-imaged and gradeable images obtained

Study Treatment Group 4: On Day 0, subjects underwent DWEK DSO and received TTHX1114 (100 ng [100 mcL]) at the time of the surgical procedure and then weekly injections×3. Subjects who have not sufficiently recovered by Day 28, received an additional dose of TTHX1114 following discussion with the Medical Monitor. Study subjects returned for follow-up on Day 1 (the day after DWEK/DSO) and subsequent study treatment administration on: Day 7, Day 14, and Day 21. Follow-up visits occurred on: Day 28* and Day 56. *Subjects, who have not sufficiently recovered by Day 28, received an additional dose of TTHX1114 following discussion with the Medical Monitor. The study subject received the 5th TTHX1114 administration, all assessments scheduled for Day 21 were repeated on Day 28 and an unscheduled visit conducted on or around Day 35 when all assessments scheduled for Day 28 were conducted.

TABLE 5
Schedule of Events Group 4
Group 4

| | Screening | Study Day | | | | | | | | | |
|---|---|---|---|---|---|---|---|---|---|---|---|
| | (≈28 | | | 7 | 14 | 21 | 28 | 56 | 84 | 168 | 336* |
| | days) | 0* | 1 | (±1) | (±1) | (±1) | (±3) | (±3) | (±21) | (±21) | (±21) |
| Informed Consent | X | | | | | | | | | | |
| Demographics | X | | | | | | | | | | |
| Medical and Ocular History | X | | | | | | | | | | |
| Comprehensive Ocular | SE | | | | | | SE | SE | | | SE |
| Examination (including slit | | | | | | | | | | | |
| lamp examination and | | | | | | | | | | | |
| dilated exam of the lens, | | | | | | | | | | | |
| fundus, vitreous and | | | | | | | | | | | |
| MAC-OCT | | | | | | | | | | | |
| Slit Lamp | SE^{a} | SE | SE | SE | SE | SE | SE^{a} | SE^{a} | | | SE |
| Specular Microscopy (incl | SE | | | | | | SE | SE | SE | | SE |
| peripheral images at Screening) | | | | | | | | | | | |
| Assessment of Corneal | | SE | | SE | SE | SE | SE | SE | SE | SE | SE |
| Thickness^{b} | | | | | | | | | | | |
| Pentacam | | SE | | | | | SE | SE | | | |
| Best-corrected Visual | SE | SE | | | SE | SE | SE | SE | SE | SE | SE |
| Acuity | | | | | | | | | | | |
| Intraocular Pressure (IOP) | SE | | SE | | | | SE | SE | | | |
| measurement | | | | | | | | | | | |
| Pre-injection/procedure | | SE | | SE | SE | SE | | | | | |
| Post-injection | | SE | | SE | SE | SE | | | | | |
| Urine pregnancy test | X | | | | | | X | | | | |
| (WOCBP) | | | | | | | | | | | |
| Plasma for ADA | | X | | | | | X | | | | X |
| Pre-injection | | | | | | | | | | | |
| Plasma for | | | | | | | X | | | | |
| Pharmacokinetics (PK) | | | | | | | | | | | |
| Pre-injection | | X | | | | | | | | | |
| 1.5 hours after injection | | X | | | | | | | | | |
| Optional Blood for Genetic | | X | | | | | | | | | |
| Testing | | | | | | | | | | | |
| V-FUCHS | X | | | | | | X | X | | | |
| DWEK/DSO | | X | | | | | | | | | |
| Collect endothelium | | X | | | | | | | | | |
| tissue sample | | | | | | | | | | | |
| Collect Aqueous Humor | | X | | | | | | | | | |
| sample | | | | | | | | | | | |
| Study Treatment IC | | 100 | | 50 | 50 | 50 | | | | | |
| Injection (SE) | | ng | | ng | ng | ng | | | | | |
| Concomitant Medications | X | X | X | X | X | X | X | X | | | |
| Review | | | | | | | | | | | |
| Assess Adverse Events | | X | X | X | X | X | X | X | | | |

OU = both eyes,
SE = Study Eye,
V-FUCHS = Visual Function and Corneal Health Status
Note:
Unless otherwise specified assessments to be performed prior to study treatment injection, if applicable
*Day 0 pre-treatment ocular assessments may be obtained within the 7 days prior to Day 0 (if screening visit assessments were within 7 days, they do not need to be repeated)
^{a}as part of Comprehensive Eye Examination
^{b}measured by AS-OCT (preferred) if AS-OCT not available, Pentacam or Ultrasound Pachymetry is to be used
(Note:
subjects must be assessed using the same modality throughout the study)
^{c} IOP pre-injection and then post-injection every 30 to 60 minutes until less than at least 5 mmHg higher than pre-injection IOP
(Note:
IOP may not be measurable post injection on Day 0)
*End of Study Visit (EOS) may be performed any time after Day 168; if the Day 336 visit, if the subject has already completed the study, the subject should be contacted and asked to provide a plasma sample for ADA; any subject who did not have gradeable specular images at their most recent visit should be re-imaged and gradeable images obtained

Long-term Follow-up (All Treatment Groups): A subject underwent long-term follow-up for up to 1 year following the later of DWEK/DSO or the first TTHX1114 administration. Long-term Follow-up visits occurred on: Day 84, Day 168, and Day 336/End of Study Visit. For subjects who have had both eyes treated on study, the Day 168 and Day 336 visits for both eyes were combined calculated based on the date of surgery of the second eye. Day 336 was considered the End of Study Visit and included a Comprehensive Eye Examination.

End of Study Visit: An End of Study Visit (EOS) was performed on all subjects. The EOS was conducted any time after Day 168, during the EOS assessments scheduled for Day 336 was performed and a plasma sample for Anti-Drug Antibody (ADA) analysis was collected. If the subject has already completed the study, the subjects should be contacted and asked to provide a plasma sample for ADA. Any subject who did not have gradable specular images at their most recent visit should be re-imaged and gradable images obtained. It is important that gradable specular images were collected for all subjects at the subjects' last visit.

### Efficacy Endpoints

Efficacy endpoints in this study include: (1) The number of subjects in each group with 20/40 or better Best Corrected Visual Acuity (BCVA) at Day 28; and (2) Central corneal thickness at Day 28.

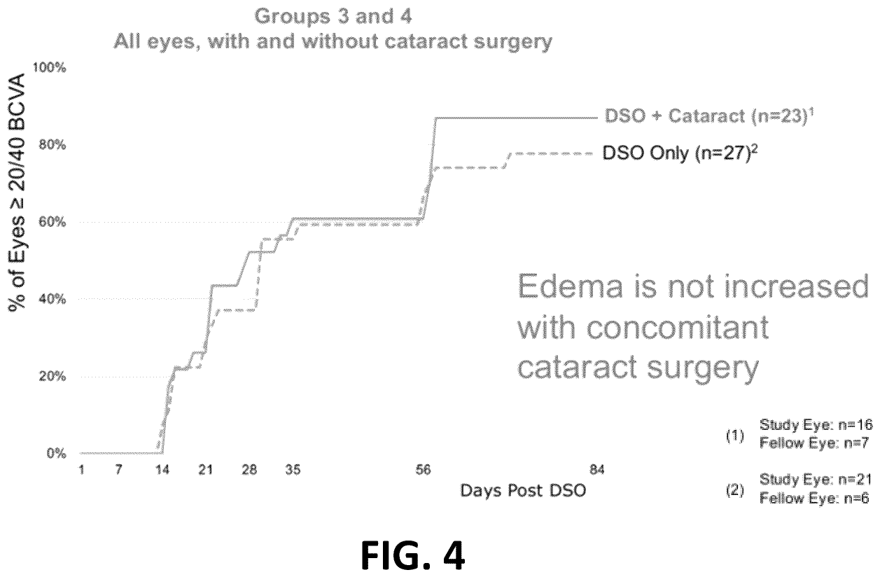

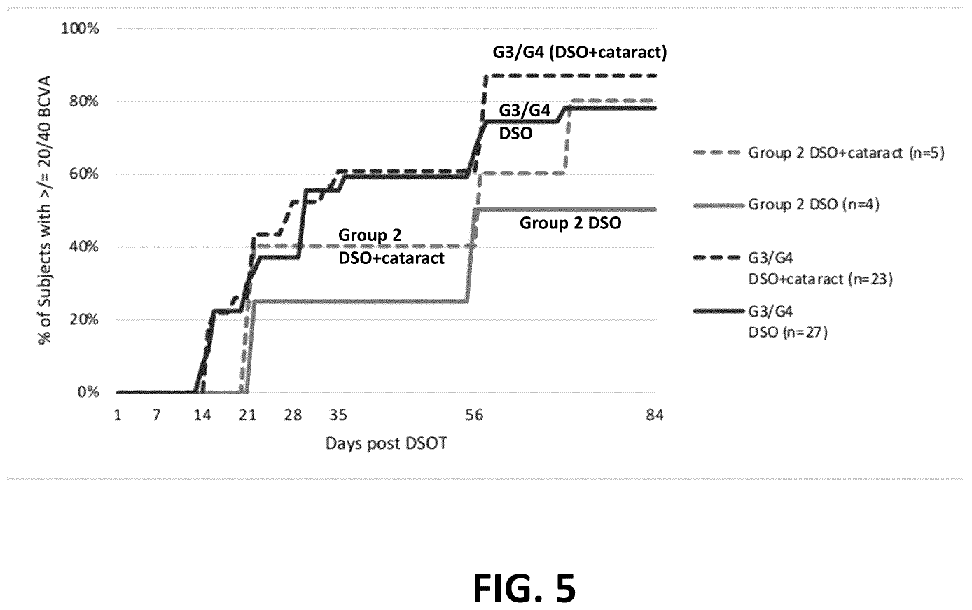

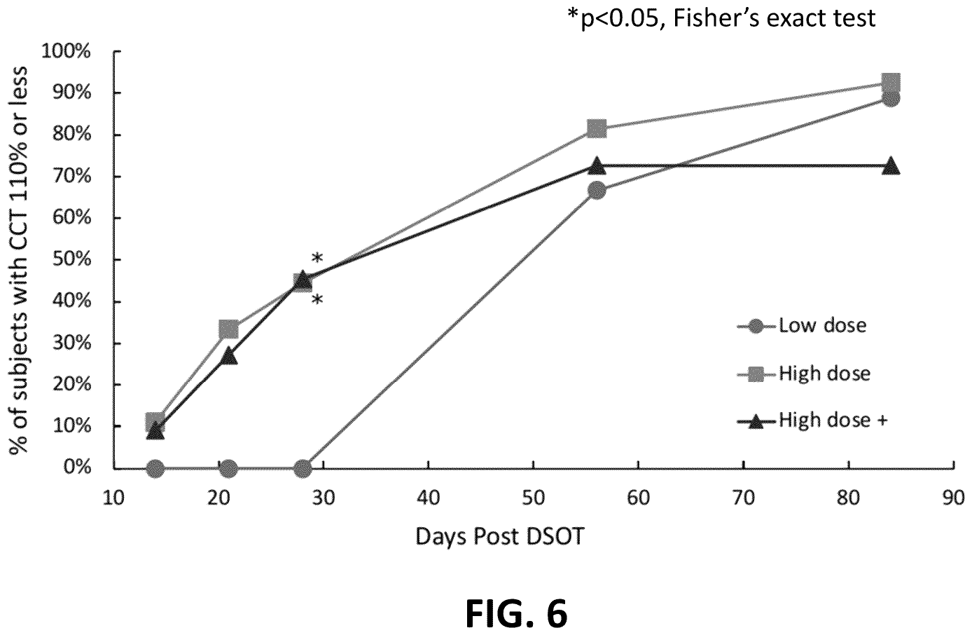

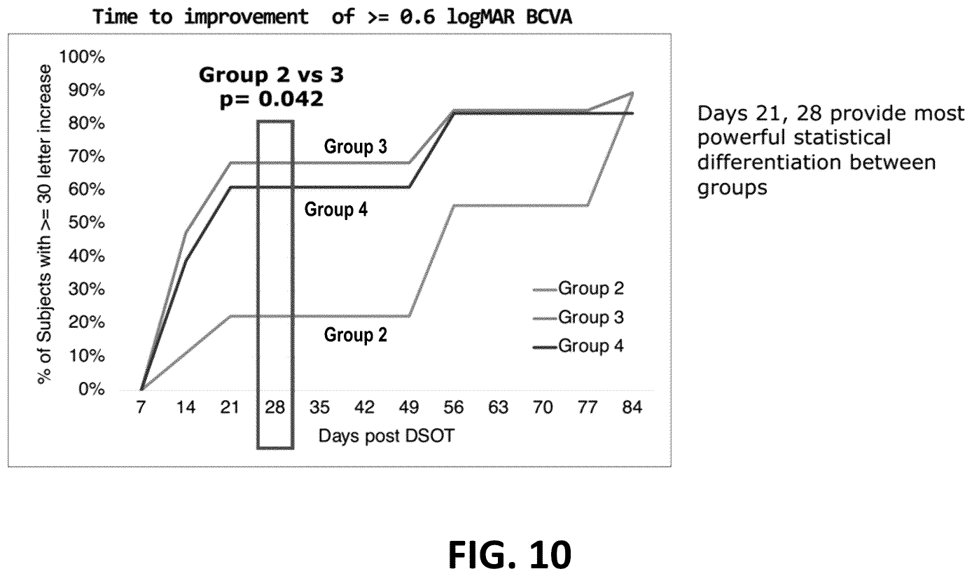

The following additional endpoints was also assessed: (1) The number of subjects in each group with 20/40 or better BCVA at Day 21, 56 and 84 (FIG. 4 & FIG. 5); (2) Early treatment diabetic retinopathy study (ETDRS) letter scores at Day 21, 28, 56 and 84; (3) Time to a BCVA of 20/40 over the course of the study; (4) Central corneal thickness at Day 21, 56 and 84 (FIG. 6); (5) Visual Function and Corneal Health Status (V-FUCHS) at Day 28 and 56; (6) Change from Baseline in intraocular pressure over the course of the study; (7) Change from pre- to post-injection in intraocular pressure on all injection days; (8) Slit-lamp examination at Day 28, 56 and 84; and (9) Central and peripheral CEC counts over the course of the study. FIG. 10 shows proportion of subjects/eyes with increase of at 0.6 log MAR (6 lines, 30 letters) BCVA from the first post-surgical visit by group by visit. Comparison of group 2 vs group 3 by Fisher's exact test.

FIG. 4 shows analysis of the proportion of all eyes in group 3 and 4 pooled for eyes that received Descemet Stripping Only (DSO) only (dashed line) or DSO+cataract surgery (solid line) by visit. Accelerated recovery of vision maintained despite cataract surgery. FIG. 5 shows analysis of the proportion of all eyes in group 2 and in groups 3 and 4 pooled for eyes that received DSO only or DSO+cataract surgery by visit. Visual outcome in high-risk patients improved regardless of cataract surgery.

The study allowed treatment of the contralateral eye. To avoid ambiguity the eye that was treated first was referred to as the Study Eye (SE); the contralateral eye was referred to as the Fellow Eye (FE).

### Measures Taken to Minimize/Avoid Bias

All adverse events were recorded regardless of suspected causality. Additionally, subjects were assigned to the open treatment group at the time of determination of eligibility dependent upon the subject's desire to receive investigational product. All subjects in all treatment groups are required to meet all eligibility criteria. All primary and secondary efficacy endpoints were source data verified or provided by an independent 3rd party (e.g., central reader).

Study Treatment: Study treatment (TTHX1114) was administered as an intracameral injection to subjects in Treatment Group 2.

### TTHX1114 Drug Product

TTHX1114 drug product is a solution formulated in phosphate buffered saline manufactured in a contract sterile fill/finish facility in compliance with cGMP regulations. TTHX1114 was provided in a concentration of 1.0 ng/mcL.

TTHX1114 was provided in cartons of 5 vials. The outer box of the kit contained the kit number, storage conditions, and the following statement “Caution: New Drug—Limited by Federal (or United States) Law to investigational use.” Each vial had a label affixed to it with the lot number.

### TTHX1114 Storage

TTHX1114 was shipped frozen (on dry ice) and was stored at −20° C. in a secure area. Excursions from temperature limits was reported to the Trefoil (or designee) to determine whether the affected vials can be used. Access to investigational product was limited to individuals authorized by the Investigator to dispense the study treatment.

### TTHX1114 Preparation

Vials was thawed prior to study treatment administration and gently mixed by inversion. Study treatment preparation was started (i.e., allowed to thaw at room temperature; NB: do not apply external heat) after the subject has arrived in the clinic to undergo study treatment. All study treatment injections were administered within 8 hours of the start of thawing of the study treatment vial.

A low-volume syringe was supplied for each study treatment administration. In order to accurately administer the precise volume of study treatment, the JuvaPen® device was provided for administering dose volumes less than 30 mcL.

### Duration of the Study and Study Treatment

Each subject was expected to be on study for approximately 1 year following DWEK/DSO.

TABLE 8

| | | | Optional Extended | |
|---|---|---|---|---|
| | Main Study | | Dosing Period | |
| | # of | Duration | # of | Duration |
| | injections | of Treatment | injections | of Treatment |
| Group 1 | — | — | — | — |
| Group 1a | 5 | over 28 days | — | — |
| Group 2 | 5 | over 24 days | | |
| Group 3 | 1 | one time | 4 | over 21 days |
| Group 3a | 4 | over 21 days | — | — |
| Group 4 | 4 to 5* | over 21 to 28 days | 1* | one time |

*Subjects who received 5 injections in Group 4 are NOT eligible for Optional Extended Dosing Period (OEDP)

Stopping Rules: Stopping rules for the study was the incidence of unacceptable toxicity. This was an open-label study, and the frequency and severity of all adverse events were monitored on an ongoing basis.

### Investigational Product Accountability

In accordance with 21 CFR 312.61 and 21 CFR 312.62(a), the Investigator (1) administered the drug only to subjects under the investigator's personal supervision or under the supervision of an investigator responsible to the investigator, (2) did not supply the investigational drug to any person not authorized under this part to receive it, and (3) maintained adequate records of the disposition of the drug, including dates, quantity, and use by subjects.

If the investigation was terminated, suspended, discontinued, or completed, the investigator returned the unused supplies of the drug to the sponsor, or otherwise provided for disposition of the unused supplies of the drug under 21 CFR 312.59.

Data to be Entered into the Database

The majority of data collected was entered into the database via the EDC system. Data from 3rd parties (e.g., central labs or reading facilities) was imported into the clinical database, these data included: (1) Endothelial cell counts and characteristics assessed by the central reading center, (2) Pharmacokinetic measurements, (3) Anti-drug antibody assay results, and (4) Clinical laboratory testing results.

Imported data was reconciled (e.g., Subject ID, date, time, timepoint) with the clinical database and the results was uploaded into the database under a Quality and Data Transfer Specifications Agreement. Uploaded data was 100% quality checked with the data at the source.

Other data and results generated by the investigational site were data entered directly into the electronic data capture (EDC) system. All data in the EDC was monitored and critical variables were 100% source data verified.

### Selection and Withdrawal of Subjects

Potential subjects were identified on the basis of having a confirmed diagnosis of Fuchs Endothelial Corneal Dystrophy (FECD) and being a suitable candidate for DWEK/DSO. Most potential subjects came directly from the patients receiving care at the investigational site. All subjects were suitable for DWEK/DSO procedure independent of the clinical trial.

Inclusion Criteria: (1) Male or female, 18 years of age or older; (2) Scheduled for DWEK/DSO with a planned central descemetorhexis of approximately 4 mm to 5 mm; (3) Subjects who are women of childbearing potential (WOCBP) must be using an acceptable method of birth control; (4) Proposed Study Eye that meets the following criteria: a. Fuchs Endothelial Cell Dystrophy (FECD) with central guttae deemed to be a contributing cause of visual symptoms (i.e., decreased visual acuity from the cornea as evidenced by morning blur and/or central corneal edema). Non-confluent peripheral guttae is not exclusionary: b. Clear peripheral cornea with adequate CEC reserve or based on other criteria such as CEC characteristics: c. No sub-epithelial haze/fibrosis that would interfere with postoperative visual acuity: d. No history of the following-Secondary corneal pathology, Refractive surgery in the study eye, Extra-ocular inflammation from any non-infectious or infectious cause (bacterial, viral or fungal) in the study eye within the 6 months prior to Study Day 0 (Note: Mild blepharitis and/or dry eye related inflammation are allowed): e. No condition that would impair examination of the anterior chamber structures; (5) Subjects receiving TTHX1114 must have a Fellow Eye with adequate function (i.e., vision of 20/100 or better).

To be eligible for the Optional Extended Dosing Period (OEDP); study subjects must have visible central endothelium by specular microscopy on Day 168.

Exclusion Criteria: (1) Planned post-operative rho-kinase inhibitor use; (2) History of: Ocular cancer (including melanoma), Herpetic keratitis, Documented and repeated elevated IOP in either eye (Note: well-controlled glaucoma is allowed), Posterior Polymorphous Corneal Dystrophy (PPCD; aka Schlichting dystrophy), Uveitis, or Intolerance, hypersensitivity, or significant allergy to any drug compound, food, or other substance (Note: This includes all components and excipients of the study drug); (3) Current or recent (e.g., the 28 days prior to Study Day 0) participation in any other interventional clinical research study; (4) Use of any of the following in the 3 months prior to Study Day 0, Systemic or dermatological cytotoxic chemotherapy or Rho-kinase inhibitor; (5) Use of any of the following in the 1 month (28 days) prior to Study Day 0, Cyclosporine ophthalmic emulsion, (e.g., RESTASIS®), Xiidra® (lifitegrast ophthalmic solution), or Systemic (nasal, inhaled, oral, parenteral, or topical) corticosteroid (Note: Potential subjects on a stable chronic low dose that is not expected to change in the 3 months following Study Day 0 may be considered for the study after approval by the Medical Monitor); (6) Use of hypertonic saline eye drops (e.g., Muro 128) within the 4 days before Study Day 0; (7) Women of childbearing potential who are currently pregnant, breast feeding, planning to become pregnant during the study, or not willing to use highly effective birth control measures; (8) An employee of the site that is directly involved in the management, administration, or support of this study or be an immediate family member of the same; (9) Any other reason (e.g., serious systemic disease or uncontrolled medical condition) that, in the opinion of the Investigator could increase the subject's risk, interfere with the interpretation of the study results, or affect the subject's ability to provide informed consent or comply with the study

### Guidance Re Exclusion of Intolerance, Hypersensitivity, or Significant Allergy:

Patients who have a history of hypersensitivity and/or hyperimmune responses were not included in this study but potential subjects with a history of mild non-significant allergies (e.g., antibiotic allergies without a respiratory component) were allowed at the discretion of the Investigator. The determination of “significant” was intentionally left to the discretion of the Investigator. To provide additional guidance specific to the definition of “significant”, please utilize the NCI CTCAE criteria of Grade 3 or higher as “significant.”

Grade 1 represents Systemic intervention not indicated.

Grade 2 represents Oral intervention indicated.

Grade 3 represents Bronchospasm; hospitalization indicated for clinical sequelae; intravenous intervention indicated.

Grade 4 represents Life-threatening consequences; urgent intervention indicated.

Therefore, anyone who has had an allergic response to any substance that resulted in bronchospasm, hospitalization, and/or parenteral intervention was excluded from this study. Known allergic sensitivity to any component of TTHX1114 (regardless of severity) was exclusionary.

As a reminder, TTHX1114 ingredients comprise sodium chloride, potassium phosphate monobasic, sodium phosphate di-basic anhydrous, recombinant human albumin solution, or polysorbate 80.

### Subject Withdrawal Criteria

Subjects were discontinued from continued study treatment if any of the following occurred: Intolerable side effects, The Investigator feels it is in the subject's best interest, Non-compliance, or Pregnancy

Subjects who were discontinued from study treatment were expected to remain on the study and completed all appropriate safety and efficacy assessments.

Subjects might withdraw from the trial at any time and for any reason.

If a subject withdrew from the trial, attempts were made to contact the subject to determine the reason(s) for discontinuation while respecting the subject's privacy. All procedures and evaluations required by the final study visit were completed in the event of early withdrawal. All subjects who discontinued the trial due to an adverse event were followed until the event has resolved, returned to baseline, or stabilized (if resolution is not anticipated).

### Treatment of Subjects

### Subject Screening and Enrollment:

Potential study participants were asked to sign an Informed Consent Form (ICF) prior to any study assessments. Screening was completed within approximately 28 days of Study Day 0 (unless approved by the Medical Monitor); non-qualifying screening assessments (e.g., specular microscopy) and screening assessments outside this window was repeated. Screening assessments included: Assessment of study eligibility, Medical and ocular history, BCVA assessment, Comprehensive ocular examination (including slit lamp biomicroscopy and dilated examination of lens, fundus, and vitreous), and Specular microscopy (including images of the central and peripheral cornea).

Descemetorhexis without Endothelial Keratoplasty (DWEK)/Descemet Stripping Only (DSO): Patients with FECD who had a planned DWEK/DSO or DMEK procedure were evaluated for eligibility in this study. As the DWEK/DSO procedure was not a study procedure DWEK/DSO was done according to institutional policy with a descemetorhexis area of 4 to 5 mm. The method of surgery included the use of the Sinskey Hook (with or without forceps) or an irrigation/aspiration (I&A) tip.

Investigators were selected based on their demonstrated expertise (e.g., historical number of procedures and relative success rate). The Lead Investigator assisted in the selection of investigational sites and the vetting of Investigators. Only qualified and vetted ophthalmologists performed the surgical procedure on subjects enrolled in this study to minimize operator variability. Subjects with a descemetorhexis area of >5 mm were excluded from efficacy analyses but were included in the safety analyses.

AS-OCT was performed at least 1 time following DSO (e.g., Day 28) to confirm the size of the descemetorhexis area, especially in subjects with delayed recovery.

The Study Eye (SE): The Study Eye (SE) was determined by the Investigator prior to the clinical trial as eligibility for the study required a planned DWEK/DSO procedure planned for the medical management of the patient. The SE was defined as the eye that was selected by the treating physician for surgical intervention of DWEK/DSO and the eye to which TTHX1114 was administered (if applicable).

If a study subject wishes to undergo DWEK/DSO in the Fellow Eye after the SE has recovered, the subject was re-enrolled in Group 3 or Group 4 and the contralateral eye was treated. “Recovery” was based on the Investigator discretion following the primary efficacy endpoint evaluation on Day 28 (e.g., BCVA of at least 20/40 or have a measured CCT of less than a 10% increase from baseline).

### Study Treatment Administration/Intracameral Injection:

This procedure was performed by a qualified and trained ophthalmologist who was familiar with DWEK/DSO and who had been trained on TTHX1114 administration.

Materials: (1) A standard eyelid speculum, (2) Povidone iodine solution (5% or 10%), (3) Balanced salt solution (BSS) flush, (4) Topical fluoroquinolone antibiotic such as: Gatifloxacin ophthalmic solution 0.5% and Moxifloxacin hydrochloride ophthalmic solution 0.5%, In cases of allergy or contraindication to fluoroquinolone, Polytrim® (polymyxin B sulfate and trimethoprim ophthalmic solution, USP) may be used, (5) Topical anesthetic (at the discretion of the Investigator), (6) 30-gauge needle, (7) 1-mL syringe (company-provided), (8) Study treatment (TTHX1114), (9) Sterile cotton-tip applicator, (9) Additional materials if required by institutional policy.

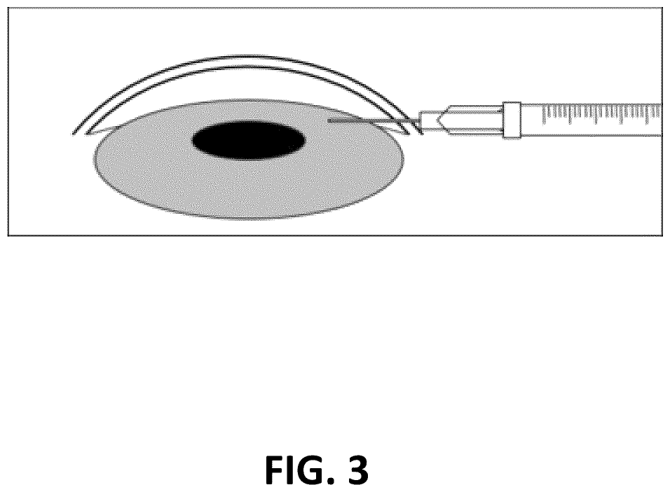

Procedure Non-surgery days: After appropriate sterile prepping and draping,
1. Apply topical antibiotic to the study eye
2. Instill topical anesthetic
3. Cleanse the periorbital area (including eyelashes and lid margins) with povidone-iodine (5% or 10%)
4. Flush the eye with povidone-iodine (5% or 10%)
   a. Flush with balanced salt solution (BSS) following povidone-iodine (at discretion of Investigator)
5. Ensure adequate anesthesia
6. Insert the eyelid speculum
7. Stabilize the eye
8. Administer study treatment (FIG. 3)
   a. Insert the needle with bevel side down into the corneoscleral limbus
      i. The 3 o'clock- and 9 o'clock-positions are recommended for best access to the cornea (especially in patients with deep orbit, prominent eyebrow, and/or prominent nose that may interfere with access)
      ii. Avoid areas of previous corneal/scleral or paracentesis incision sites
   b. Slowly advance the needle parallel to the iris plane bevel-side down to avoid risk to the iris and/or the lens
   c. Stop when the anterior chamber has been entered near the periphery
      i. Avoid the center of the eye if possible
   d. Deliver the dose of study treatment of the syringe holder lever
   e. When study treatment delivery is complete, remove the needle from the eye
9. Apply a sterile cotton-tip applicator to the injections site
   a. Avoid extensive massage of the eyelid or ocular surface
10. Monitor IOP 30 to 60 minutes after IC injection to ensure that the post-treatment IOP is less than 5 mmHg higher than the pre-injection IOP measurement. Continue to monitor at least every 30 minutes until the post-injection IOP is within 5 mmHg of the pre-injection IOP measurement
11. Apply topical antibiotic to the study eye prior to discharge
12. Instruct the subject to self-administer topical antibiotic to the study eye 2 more times at home (e.g., 6:00 PM and bedtime)

### Procedure Study Day 0:

Following surgical closure of the eye, the eye was not fully inflated, and intraocular pressure was low and possibly not measurable. Following completion of the DWEK/DSO procedure, TTHX1114 was administered as soon as practicable (described in Step 8 above) and standard post-operative care including Steps 9 to 12 above was followed, as appropriate.

The Investigator administered/prescribed post-procedure steroid eye drops (e.g., prednisolone acetate [1% ophthalmic suspension], qid), if clinically indicated or at his/her discretion.

### Timing and Scheduling of TTHX1114 Administration:

For Group 2: The Day (−3) injection was administered within approximately 48 to 72 hours prior to the planned Day 0 (Day of DWEK/DSO).

For Group 2, Group 3, and Group 4: The Day 0 (Day of DWEK/DSO) injection was administered at any time after the completion of DWEK/DSO.

For Groups 1a and 3a: (1) The Day 0 injection was administered after approximately 56 days following the day of DWEK/DSO, after discussion with and approval by the Medical Monitor (or designee); (2) Initiation of TTHX1114 was moved earlier based on the observed time to response in the subjects in Group 2.

### Subsequent TTHX1114 Administrations:

Following the Day 0 TTHX1114 administration, subsequent weekly administrations occurred approximately every 7 days±1 day. If a study treatment administration could not be administered within the 6 to 8 days following the prior administration, the study treatment was delayed, and the scheduling of all remaining administrations adjusted to ensure that TTHX1114 was not administered fewer than 5 days after the prior administration.

### Concomitant Medications and Treatments:

Concomitant Medications: Subjects were expected to receive concomitant medications for the management of concurrent medical conditions. Medications taken during the 28 days prior until 28 days following the last study treatment administration were recorded. Artificial tears were allowed during the study, but other ocular medications was discussed with the masked Medical Monitor.

Prohibited Medications: The following medications were prohibited unless approved by the Medical Monitor; (1) Rho-kinase inhibitors (topical or systemic, e.g., Fasudil, Netarsudil [Rhopressa]), (2) Systemic or dermatological cytotoxic chemotherapy, (3) Cyclosporine ophthalmic emulsion, or (4) Xiidra® (lifitegrast ophthalmic solution). Subjects requiring the use of prohibited medications must be discussed with the Medical Monitor.

Pregnancy Testing and Contraception: Women of child-bearing potential (WOCBP) are defined as a premenopausal female capable of becoming pregnant. Women who are not WOCBP are those who have not had a menstrual cycle for at least 2 years or who have had hysterectomy, bilateral tubal ligation, or bilateral oophorectomy. Sexually active female subjects who are WOCBP must use at least 1 highly-effective method of contraception or at least 2 less effective methods of contraception (including 1 barrier method), starting at least 1 month prior to Day 0, and male subjects who are sexually active with women of childbearing potential must agree to use a double-barrier method of contraception. Per ICH M3(R2) Non-Clinical Safety Studies for the Conduct of Human Clinical Trials and Marketing Authorization for Pharmaceuticals, highly-effective methods are defined as “those, alone or in combination, that result in a low failure rate (i.e., less than 1% per year) when used consistently and correctly.”

### Study Procedures:

Demographics: Demographic information was recorded, including age, sex, race, and ethnicity. The subject's height and weight were measured and recorded.

Medical History and Ocular History: During the Screening/Pretreatment Period, the Investigator (or designee) reviewed the medical history of all potential subjects to document all relevant clinically significant baseline medical conditions, concomitant medication use, and to determine study eligibility. Medical History Assessment included review of all available medical records and patient interview. Assessment of all eligibility criteria was evidenced in the study records. Medical history included a detailed disease history regarding onset, severity, and history of prior therapy.

In addition to the data collected in demographics and medical history, specific information was collected regarding each subject's ocular history, including: (1) present condition of duration (date/age of onset), single or both eyes involved, and associated symptoms; (2) other corneal dystrophies; (3) contact lens use and history; (4) past ocular surgery (including lasik); (5) history of ocular trauma; (6) family history of ocular conditions; (7) allergies; (8) diabetes; (9) hypertension; and (10) autoimmune disease. Additional relevant medical history was also recorded.

Comprehensive Ocular Examination: A comprehensive ocular examination included slit-lamp biomicroscopy and a dilated examination of the lens, fundus (retina, optic disc, macula, fovea, and posterior pole), and vitreous. The comprehensive ocular examination also included baseline macular optical coherence tomography (Mac-OCT). As BCVA is the primary efficacy measurement in the study, it was important to identify any macular pathology or other confounding co-morbidities.

Slit Lamp Biomicroscopy: The slit lamp biomicroscopy examination was performed using a beam of 1.0 mm height and 1.0 mm width with the beam at maximum luminance and using the high-powered lens. The subject was seated during the examination. This procedure was conducted in the same manner for all subjects and included an assessment of each of the following: (1) lens, (2) corneal edema (assessed in the central region), (3) size of corneal edema region (in mm), (4) corneal opacity, (5) stromal clarity (per CPTS grading scale), (6) conjunctival hyperemia, (7) sclera-ciliary flush, (8) anterior chamber cells, and (9) anterior chamber flare. These assessments were graded according to the criteria described in Table 9.

TABLE 9
Grading of Slit Lamp Biomicroscopy Assessments

| | Corneal | | | Sclera | Conjunctival |
|---|---|---|---|---|---|
| | Edema | Corneal | Stromal | Ciliary | Hyperemia |
| Grade | (central) | Opacity | Clarity | Flush | (vasodilation) |
| 0 | Absent | Absent | Clear | Absent | Absent |
| 1 | Mild | Mild | Equivocal | Mild | Some |
| 2 | Moderate | Moderate | Cloudy | Moderate | Moderate |
| 3 | Severe | Severe | Opaque | Severe | Excessive |

| | Anterior | | Anterior |
|---|---|---|---|
| Grade | Chamber Cells | | Chamber Flare |
| 0 | No cells seen | | None |
| 0.5+ | 1-5 | cells | — |
| 1+ | 6-15 | cells | Faint |
| 2+ | 16-25 | cells | Moderate (iris and |
| | | | lens detail clear) |
| 3+ | 26-50 | cells | Marked (iris and |
| | | | lens details hazy) |
| 4+ | >50 | cells | Intense (fibrin or |
| | | | plastic aqueous) |

Specular Microscopy: Specular microscopy images were obtained of the periphery and center of the cornea. The center of the cornea was defined as the region of the cornea above the pupil. If a subject was to be dilated at a visit, specular microscopy was performed prior to dilation. To address differences in location of the image within a given area of the cornea, five acceptable images were taken at each required timepoint. Peripheral images were collected at the screening visit; central images were collected at prior to treatment and whenever possible following treatment. Due to the nature of corneal edema following DSO, specular microscopy images were not able to be obtained at all post-operative timepoint prior the deturgescence of the cornea. Attempts were made to collect images at all timepoints; if images were unable to be obtained, the reason for not being able to collect images was recorded. If specular microscopy images were obtainable, it was expected that images remained obtainable at all subsequent timepoints. Prior to the end of each specular microscopy session, the technician reviewed the images to ensure that the captured images are of the highest quality obtainable. Subjects with inadequate images were required to return to the investigational site for repeat imaging as an unscheduled visit. Endothelial cell density, percent hexagonality and the coefficient of variation were assessed from specular microscope images. Cornea Image Analysis Reading Center (CIARC) at University Hospitals Cleveland Medical Center functioned as the centralized reading center and was responsible for the certification of equipment and training of technicians as well as reading images and conducting image analysis. If more than one specular microscope was installed at a site, the same specular microscope was used for all visits for a given patient. Baseline images and on-study central images were sent to CIARC; baseline images were archived, and on-study images were assessed for efficacy.

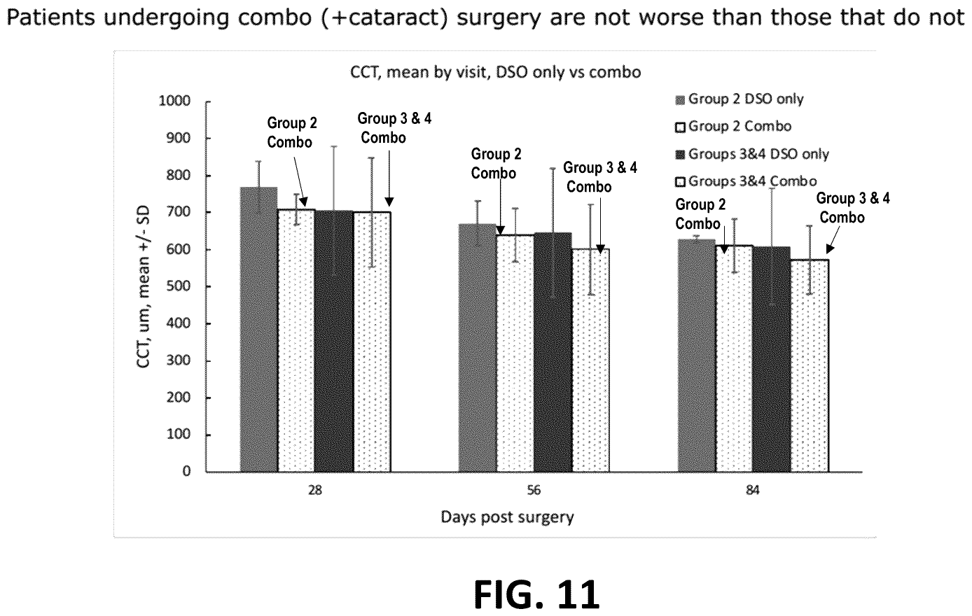

Central Corneal Thickness Assessment: Anterior segment optical coherence tomography (AS-OCT) is the preferred method of measuring corneal thickness. If AS-OCT was not available, Pentacam, or ultrasound pachymetry were used to measure central corneal thickness (CCT). Other reliable methods of measuring CCT (e.g., LENSTAR Optical Biometer) were used if approved by the Medical Monitor. If ultrasound pachymetry was used as the method of measurement, the measurements were obtained using a pachymeter that was approved by Trefoil (or designee); automated “pachymetry” readings obtained using the specular microscope were not acceptable. The same methodology of assessing CCT was used consistently within a subject, whenever possible. If an alternate method was used, the reason for the alternative method was reported. CCT was measured by several modalities at each timepoint (FIG. 6-FIG. 8 & FIG. 11-FIG. 14).

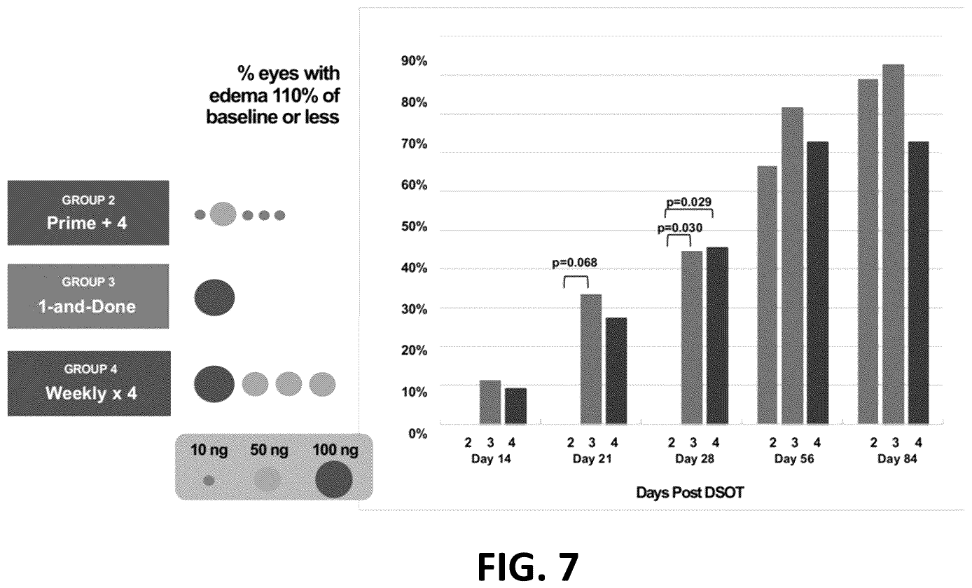

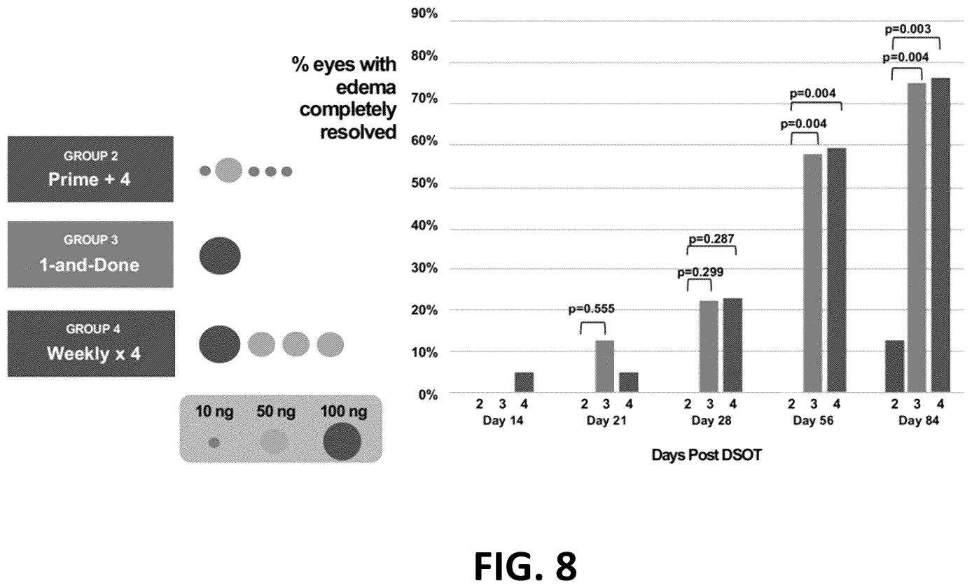

FIG. 6 shows proportion of subjects/eyes with central corneal thickness not more than 10% above baseline (Central Corneal Thickness (CCT) 110% of baseline or less) by visit by group. Differences between group 2 (low dose) and groups 3 and 4 (high dose) are statistically significant with p<0.05 at day 28, Fisher's exact test. TTHX1144 accelerates edema resolution after endothelial damage (DSO). FIG. 7 shows proportion of subjects/eyes with central corneal thickness not more than 10% above baseline (CCT 110% of baseline or less) by visit by group. Differences between group 2 (low dose) and groups 3 and 4 (high dose) are statistically significant with p<0.05 at day 28, Fisher's exact test. Dose response was shown for central corneal thickness. FIG. 8 shows proportion of subjects/eyes with complete resolution of corneal edema (central corneal thickness at or below baseline) by visit by group. P values, Fisher's exact test. Dose response was shown for central corneal thickness.

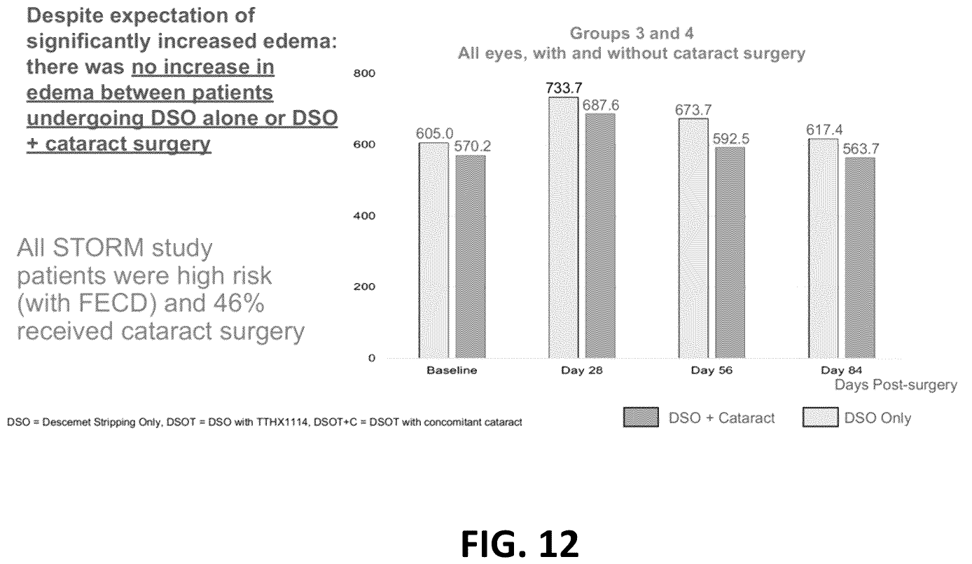

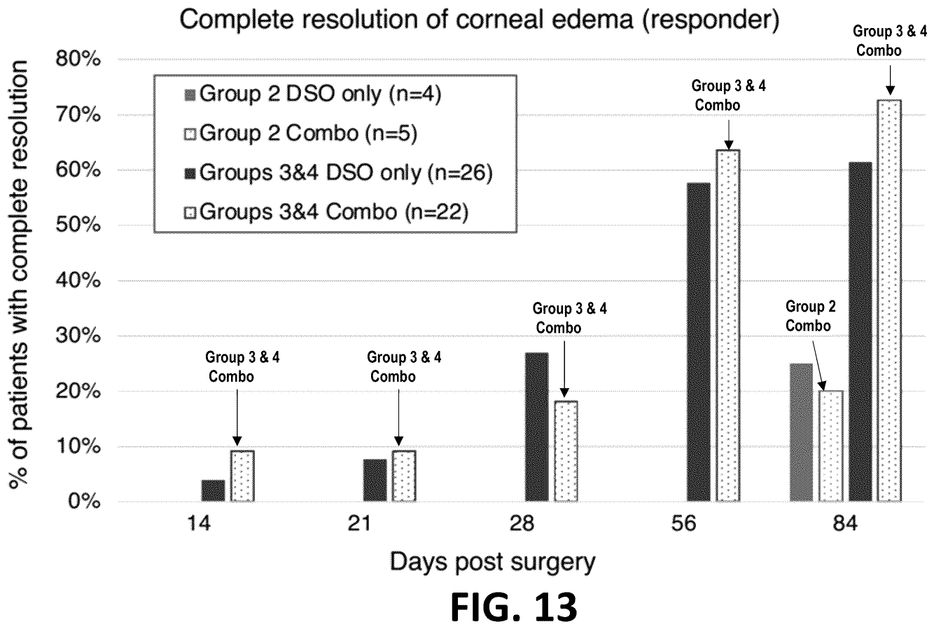

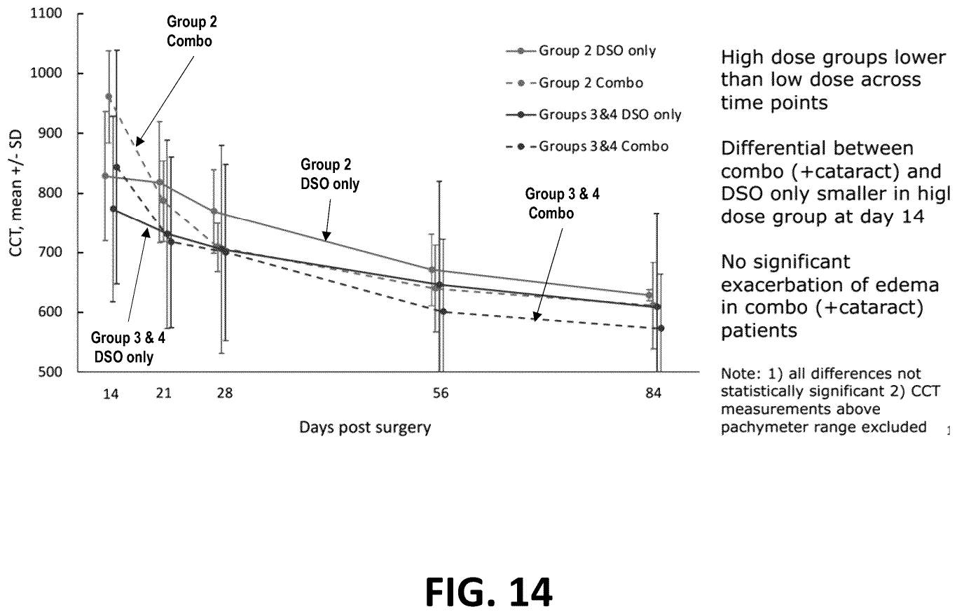

FIG. 11 shows mean corneal thickness (+/−SD) for group 2 or groups 3 and 4 pooled by visit. Solid bars include eyes that received DSO only, dotted bars DSO+cataract surgery. FIG. 12 shows mean corneal thickness in group 3 and 4 pooled by visit. Light bars include eyes that received DSO only, dark bars DSO+cataract surgery. FIG. 13 shows proportion of subjects/eyes with complete resolution of corneal edema (central corneal thickness at or below baseline) by visit by group. Solid bars include eyes that received DSO only, dotted bars DSO+cataract surgery. FIG. 14 shows mean corneal thickness in group 2 and groups 3 and 4 pooled by visit. Solid lines, eyes that received DSO only, dotted lines DSO+cataract surgery.

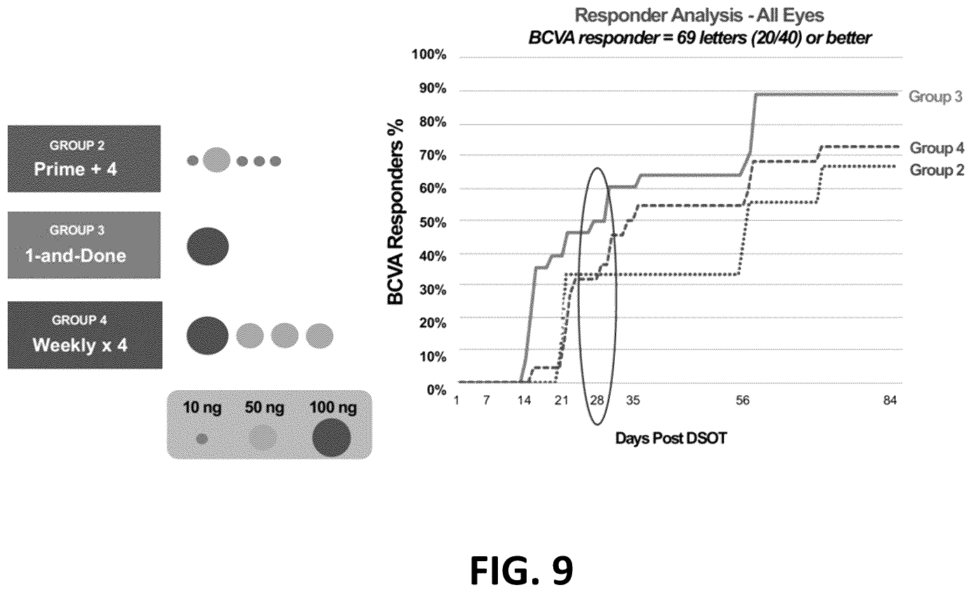

Best-corrected Visual Acuity (BCVA): Best corrected visual acuity (BCVA), was measured between 07:00 AM and 11 AM, whenever possible (unless otherwise directed) using ETDRS (Early Treatment Diabetic Retinopathy Study) charts in normal luminance lighting according to institutional practice. BCVA testing preceded any examination requiring contact with the eye, including measurement of IOP, and was performed prior to instillation of any of any dye or drops to dilate or anesthetize the eye. Refraction was performed prior to measurement of BCVA, and the appropriate correction worn in a trial frame during visual acuity testing. If a study subject did not have a BCVA of ≥20/40 (>68 ETDRS letter score) on Day 28 or Day 56, additional methods of BCVA were employed (i.e., hard lens and/or pinhole refraction) to determine if continued decreased visual acuity was due to factors other than corneal edema. An unscheduled visit was performed at the investigator's discretion prior to the next scheduled visit (FIG. 9 & FIG. 10).

FIG. 9 shows proportion of subjects/eyes with Best-corrected Visual Acuity (BCVA) of 69 letters (20/40) or better by group by visit, all eyes. FIG. 10 shows proportion of subjects/eyes with increase of at 0.6 log MAR (6 lines, 30 letters) BCVA from the first post-surgical visit by group by visit. Comparison of group 2 vs group 3 by Fisher's exact test.

Intraocular Pressure Measurement: Intraocular pressure (IOP) was measured by Goldmann Applanation Tonometry. At least two measurements were obtained and recorded (a third measurement was obtained if there was a ≥3 mmHg difference between the first two measurements). IOP was measured at least every 30 to 60 minutes following each TTHX1114 injection (except post DWEK/DSO) until the IOP was at least less than 5 mmHg higher than the pre-injection IOP. The average IOP measurement was reported at each timepoint.

Urine Pregnancy Test: WOCBP was tested prior to the start of study treatment and after the completion of study treatment to confirm a continued non-pregnant state. Commercially available urine dip-stick pregnancy tests were acceptable.

Delayed Recovery: Subjects with delayed recovery underwent additional ocular assessments at Day 28, Day 56, or at unscheduled visits. These assessments included: (1) Additional; methods of BCVA, (2) Central Corneal Thickness (by the primary or alternative method), (3) AS-OCT, (4) Pentacam, and the like. Additional ocular assessments were reported as an Unscheduled Visit.

Clinical Laboratory Assessments: Samples for clinical laboratory assessments were obtained and transmitted to a central laboratory. The laboratory parameters to be tested were summarized below in Table 10. All clinically significant laboratory results that were outside of the normal ranges were to be reported as AEs. An abnormal lab value was deemed clinically significant if either of the following conditions was met: (1) The abnormality suggested a disease and/or organ toxicity that was new or had worsened from baseline as determined by the Investigator and in consultation with Medical Monitor consult at the Investigator's request, and (2) The abnormality was of a degree that required additional active management, e.g., change of dose, discontinuation of the drug, close observation, more frequent follow-up assessments, or further diagnostic investigation. Therefore, a clinically significant laboratory value was one that suggested a new disease process, an exacerbation or worsening of an existing condition, or requires further action(s) to be taken. If a clinically significant laboratory result was found on a laboratory test (e.g., hematology), the Investigator determined if an underlying condition was the reportable AE instead of the individual laboratory parameters (e.g., anemia instead of decrease in red blood cell count, hematocrit, reticulocyte, and hemoglobin levels).

TABLE 10
Clinical Laboratory Assessments

Hematology
Hematocrit
Hemoglobin
White blood cell (WBC) count with differential
Platelet count

| Serum Chemistry | Urinalysis |
|---|---|
| Albumin | Specific gravity |
| Alkaline phosphatase | Ketones |
| ALT (alanine amino transferase)/SGPT | pH |
| AST (aspartate amino transferase)/SGOT | Protein |
| Bicarbonate/CO2 | Glucose |
| Bilirubin | Blood |
| Blood urea nitrogen | Urobilinogen |
| Calcium | Bilirubin |
| Chloride | Leukocyte esterase |
| Creatinine | Microscopic examination |
| HbA1-c | (as applicable) |
| LDH (Lactate dehydrogenase) | |
| Potassium | |
| Sodium | |
| Total protein | |
| Uric acid | |

Plasma for PK and ADA: Blood samples were collected for pharmacokinetic (PK) and anti-drug antibody (ADA) analysis at selected sites. Samples were centrifuged and/or prepared per instructions from the analytic laboratory. Pre-injection samples were collected within the 24 hours prior to study treatment administration when possible. PK samples were collected within ±5 minutes of the nominal timepoint or as soon as practicable. Following the assessment of systemic exposure (pharmacokinetics) at the 50 ng dose level, collection of PK samples was discontinued at dose levels ≤50 ng. Pre- and post-exposure ADA samples continued to be collected regardless of observed PK exposure. A plasma sample was obtained at the end of the study for ADA analysis.

Endothelium Tissue Collection: Study subjects were asked to donate their excised tissue for research purposes. A specific research consent were included in the Informed Consent Form (ICF). As soon as practicable following Study Day 0 procedure the tissue was placed in the provided transport tube (without any supernatant) and frozen/shipped to the central laboratory. These samples were collected at a subset of study sites.

Aqueous Humor Collection: Study subjects were asked to donate samples of aqueous humor obtained during the DWEK/DSO procedure on Study Day 0. Any aqueous humor that was collected was frozen/shipped to the central laboratory. When possible, aqueous humor samples were divided equally into 2 aliquots. These samples were evaluated for research purposes including endogenous levels of FGF-1. These samples were collected at a subset of study sites.

Visual Function and Corneal Health Status (V-FUCHS): The V-FUCHS is a 15-item questionnaire designed to measure patient-reported visual disability in Fuchs endothelial corneal dystrophy (FECD). Upon completion of the questionnaire, the study coordinator checked the questionnaire for completeness. Any omissions or ambiguous answers were clarified by the subject prior to leaving the clinic. The V-FUCHS was self-administered, however, trained investigational site staff administered the questionnaire to subjects who were unable to self-administer the questionnaire. Collection of V-FUCHS preceded any examination requiring contact with the eye, including measurement of IOP, and was performed prior to instillation of any of any dye or drops to dilate or anesthetize the eye (if applicable).

Optional Blood Sample for Genetic Testing: Subjects were asked to provide an optional blood sample for genetic testing. Subjects were asked to provide specific consent for this optional genetic testing. This sample was completely de-identified prior to transmittal to the testing facility. This sample was optional and participation in in the study was not conditional on the consent to provide this sample for genetic testing. The blood sample for Optional Genetic Testing was collected at any time during the study.

### Study Visits:

All study visits/assessments were conducted by qualified investigational site staff. Study visits/assessments were conducted within the protocol-specified window for each visit/assessment. If an assessment/visit was not able to be collected/conducted within the protocol-specified window, it was collected/performed as soon as practicable, and the actual date/time recorded. Comparative assessments were collected at the same time at each timepoint, when possible (unless otherwise directed). If an onsite visit was not possible, as many assessments as feasible was conducted remotely.

Screening Period/Baseline Assessments: Screening/baseline assessments were performed anytime during the Screening Period, unless approved by the Medical Monitor, except for Informed Consent (which must be obtained prior to any protocol-specific assessments).

On-study Visits: The frequency of on-study visits varied depending on the treatment group to which the study subject belongs. All study drug administration dates were at least 5 days after the prior study drug administration day (if applicable). All other visits were scheduled based on the number of days since Day 0. If a study drug administration could not be administered within the protocol window, it was administered as soon as practicable. Worksheets were provided describing which assessments were required at each visit for each treatment group.

Long-term Follow-up: Following the Day 56 evaluations, all subjects continued to be followed for long-term efficacy, durability of responses, and SAE/SAR resolution. Follow-up visits occurred approximately 3, 6, and 12 months after the DWEK/DSO procedure or until subsequent treatment in the Study Eye for FECD. Subjects were also seen as part of their regular health care, all relevant ocular assessments collected during this period were recorded.

Early Termination Visit: If a subject was discontinued from study treatment early or withdraws from the study, he/she was assessed approximately 28 days following the last study treatment administration; if the subject was unable to be evaluated approximately 28 days following the last study treatment administration, he/she was evaluated as soon as practicable (at least by telephone). All assessments scheduled for Day 28 were performed at the Early Termination Visit if Day 28 visit had not already been completed, otherwise assessments scheduled for Day 56 should have been collected.

Unscheduled Visit: Study subjects were seen at any time in between scheduled study visits at the discretion of the Investigator and any relevant study assessments performed. The date and reason for the unscheduled visit were recorded as well as the results of the relevant assessments.

End of Study Visit: An End of Study Visit (EOS) was performed on all subjects. The EOS was conducted any time after Day 168, during the EOS assessments scheduled for Day 336 were performed and a plasma sample for ADA analysis was collected. If the subject had already completed the study, the subjects were contacted and asked to provide a plasma sample for ADA. Any subject who did not have gradable specular images at their most recent visit were re-imaged and gradable images obtained. (1) comprehensive eye examination, (2) specular image collection, (3) assessment of corneal thickness, (4) assessment best corrected visual acuity, and (5) plasma sample collected for ADA

Pregnancy: Subjects was instructed to notify the Investigator as soon as possible after becoming pregnant or learning of the pregnancy of a partner. If a subject or partner of a subject became pregnant during treatment or up to 120 days following the last study drug administration, the Investigator was instructed to notify Trefoil (or designee) within 24 hours of learning of the pregnancy. If the subject became pregnant while receiving investigational product, the investigational product was permanently discontinued. Protocol-required procedures for study discontinuation and follow-up must be performed on the subject unless contraindicated by pregnancy. Other appropriate pregnancy follow-up procedures were considered if indicated. If the partner of a study participant became pregnant, the Investigator as instructed to obtain a Pregnant Partner Release Form and collect relevant information regarding the partner and the pregnancy. The Investigator discussed the risks and concerns of investigational drug exposure to a developing fetus and counsel the subject and/or pregnant partner (or ensure that such counseling is provided). Pregnancies was followed through the outcome of the pregnancy. Newborns should be followed for a minimum of 8 weeks. The Investigator completed a Pregnancy Surveillance Form and report the information regarding the pregnancy, outcome, and status of the newborn, as appropriate.

### Assessment of Efficacy

The efficacy endpoints were visual acuity and endothelial cell count/density as measures by specular microscopy. Other efficacy assessments included: (1) Slit lamp biomicroscopy assessments, (2) Central corneal thickness, and (3) IOP. The primary efficacy timepoint is Day 28.

### Assessment of Safety

### Adverse Events:

Investigators collected information related to adverse events (AEs) throughout this clinical trial. The terms and definitions were consistent with Guidance for Industry and Investigators Safety Reporting Requirements for INDs and BA/BE Studies, FDA 2012.

All AEs occurring in all subjects following exposure to study treatment until 28 days after each study treatment were reported.

AEs considered related to the study treatment by the Investigator (SARs) and SAEs were followed until resolution, return to baseline, or stabilization (if resolution is not expected).

Changes in the subject's medical condition that occur prior to the first exposure to study treatment were reported as medical history. At each post-treatment visit, subjects were asked about any possible adverse events in a non-leading manner. An example of a non-leading method of eliciting AE information was “Have there been any changes in your health since you were here last?” Subjects were also asked about the severity and/or persistence of any AEs that were ongoing at the time of their last visit.

### Adverse Events Definitions:

Adverse Events (21 CFR 312.32(a)): An adverse event (AE) is defined as any untoward medical occurrence associated with the use of a drug in humans, whether or not considered drug related. An AE (also referred to as an adverse experience) can be any unfavorable and unintended sign (e.g., an abnormal laboratory finding), symptom, or disease temporally associated with the use of a drug and does not imply any judgment about causality. An adverse event can arise with any use of the drug (e.g., off-label use, use in combination with another drug) and with any route of administration, formulation, or dose, including an overdose.

Suspected Adverse Reaction (21 CFR 312.32(a)): Suspected adverse reaction means any adverse event for which there is a reasonable possibility that the drug caused the adverse event. For the purposes of IND safety reporting, ‘reasonable possibility’ means there is evidence to suggest a causal relationship between the drug and the adverse event. A suspected adverse reaction implies a lesser degree of certainty about causality than adverse reaction, which means any adverse event caused by a drug.

Unexpected (21 CFR 312.32(a)): An adverse event or suspected adverse reaction is considered “unexpected” if it is not listed in the investigator brochure or is not listed at the specificity or severity that has been observed; or, if an investigator brochure is not required or available, is not consistent with the risk information described in the general investigational plan or elsewhere in the current application, as amended. For example, under this definition, hepatic necrosis would be unexpected (by virtue of greater severity) if the investigator brochure referred only to elevated hepatic enzymes or hepatitis. Similarly, cerebral thromboembolism and cerebral vasculitis would be unexpected (by virtue of greater specificity) if the investigator brochure listed only cerebral vascular accidents. “Unexpected,” as used in this definition, also refers to adverse events or suspected adverse reactions that are mentioned in the investigator brochure as occurring with a class of drugs or as anticipated from the pharmacological properties of the drug but are not specifically mentioned as occurring with the particular drug under investigation.

Serious Adverse Event (21 CFR 312.32(a)): An adverse event or suspected adverse reaction is considered “serious” if, in the view of either the investigator or sponsor, it results in any of the following outcomes: death, a life-threatening adverse event, inpatient hospitalization or prolongation of existing hospitalization, a persistent or significant incapacity or substantial disruption of the ability to conduct normal life functions, or a congenital anomaly/birth defect.

Important medical events that may not result in death, be life-threatening, or require hospitalization may be considered serious when, based upon appropriate medical judgment, they may jeopardize the patient or subject and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

### Adverse Events Reporting:

Adverse Event Term: Adverse events must be reported using standard medical terminology. The use of abbreviations (standard and nonstandard) should be avoided to help ensure a clear understanding of the event. An example of a standard abbreviation that may have several meanings is “MI” which could mean “myocardial infarction” or “mitral insufficiency.” All AE terms will be coded using a standardized dictionary (i.e., Medical Dictionary for Regulatory Activities [MedDRA]).

Generally, when reporting a well-known and understood condition, it is preferable to report the overall diagnosis rather that the individual signs and symptoms; the exception to this rule in this study is when the subject experiences an injection site reaction.

The term “intermittent” should be avoided as the duration and incidence of events helps in understanding the safety profile of the study drug.

Adverse Event Severity: Adverse events will be reported at the highest experienced. Adverse events severity will be graded according to the criteria described in Table 11.

TABLE 11
Severity Grading Guideline for Adverse Events

| Grade | Description |
|---|---|
| Mild | Asymptomatic or mild symptoms |
| | Clinical or diagnostic observations only |
| | Intervention not indicated |
| Moderate | Minimal, local or noninvasive intervention indicated |
| | Limiting age-appropriate instrumental ADL* |
| Severe | Medically significant |
| | Hospitalization or prolongation of hospitalization indicated |
| | Disabling |
| | Limiting self-care ADL** |

Adapted from NCI-CTCAE V5.0
Note:
A Semi-colon indicates ‘or’ within the description of the grade.
*Instrumental ADL refer to preparing meals, shopping for groceries or clothes, using the telephone, managing money, etc.
**Self-care ADL refer to bathing, dressing and undressing, feeding self, using the toilet, taking medications, and not bedridden.

Adverse Event Duration: The start date (the date that the event was first noticed) and the end date (the date that the event had completely resolved or returned to baseline) will be recorded. If the exact date is not known, the best estimate should be reported.

Adverse Event Causality: Where the Investigator assessment of the relationship of the AE to investigational product rests on medical judgment, the determination must be made with the appropriate involvement of the Investigator, or, if the Investigator is not a physician, a designated sub-Investigator who is a physician. Using the following criteria, Investigators will assess whether there is a reasonable possibility that the study treatment (drug or procedure) caused or contributed to the AE.

Related: There is a “reasonable possibility” to suggest a causal relationship between the study treatment and the adverse event based on evidence.
The Investigator should use the following criteria when assessing causality:
Is the AE a known side effect/adverse reaction to the study treatment or other therapies in this treatment class?
Is there a reasonable temporal relationship between the start of the AE and study treatment administration?
Did the AE improve when the study treatment was stopped?
Did the AE recur when the study treatment was resumed, if applicable?
Can the AE be easily attributed to a comorbid condition or concomitant medication/treatment?

Not Related: There is NOT a “reasonable possibility” to suggest a causal relationship between the study treatment and the adverse event based on evidence.

Adverse Event Seriousness: SAE criteria have the following guidelines: (1) Death: Report if you suspect that the death was an outcome of the adverse event and include the date if known; (2) Life-threatening: Report if suspected that the patient was at substantial risk of dying at the time of the adverse event or use or continued use of the device or other medical product might have resulted in the death of the patient; (3) Hospitalization (initial or prolonged): Report if admission to the hospital or prolongation of hospitalization was a result of the adverse event. Emergency room visits that do not result in admission to the hospital should be evaluated for one of the other serious outcomes (e.g., life-threatening; required intervention to prevent permanent impairment or damage; other serious medically important event); (4) Disability or Permanent Damage: Report if the adverse event resulted in a substantial disruption of a person's ability to conduct normal life functions, i.e., the adverse event resulted in a significant, persistent or permanent change, impairment, damage or disruption in the patient's body function/structure, physical activities and/or quality of life; (5) Congenital Anomaly/Birth Defect: Report if you suspect that exposure to a medical product prior to conception or during pregnancy may have resulted in an adverse outcome in the child; and (6) Other Serious (Important Medical Events): Report when the event does not fit the other outcomes, but the event may jeopardize the patient and may require medical or surgical intervention (treatment) to prevent one of the other outcomes. Examples include allergic brochospasm (a serious problem with breathing) requiring treatment in an emergency room, serious blood dyscrasias (blood disorders) or seizures/convulsions that do not result in hospitalization. The development of drug dependence or drug abuse would also be examples of important medical events.

Serious Adverse Events Reporting: Adverse events that meet the definition of serious require accelerated reporting. Investigators must report all Serious Adverse Events (SAEs) regardless of causality immediately (within 24 hours of learning of the event). If adverse events of special interest (AESI) are identified during the study, Trefoil will notify Investigators and provide instructions for reporting. Deaths occurring within 1 year of study treatment and AESIs will be reported to Trefoil in the same time frame as SAEs.

Adverse Events of Special Interest: (1) Adverse Events of Special Interest (AESI) will be reported regardless of Investigator Assessment of Causality and/or date of onset. AESIs include Neoplasms; (2) Worsening or new onset glaucoma; (3) All clinically significant ocular adverse events (e.g., need for capsulotomy). AESI will be reported as non-serious AEs and will be reviewed periodically by the Medical Monitor.

### Statistics

Sample Size: The sample size for this study was determined by clinical and practical rather than statistical considerations. Sixteen subjects in the active treatment group compared to 8 subjects in the non-treatment control group were planned during the study to provide a preliminary estimate of safety and efficacy.

Analyses: The study included a concurrent non-treatment control group. The active treatment group was compared to the control group. In addition, descriptive statistics were used to tabulate and summarize other efficacy variables and safety outcomes. Baseline and demographic characteristics were presented. Continuous variables were summarized by descriptive statistics (sample size, mean/standard deviation, median, minimum and maximum). Discrete variables were summarized by frequencies and percentages. Ocular AEs involving the study eye were summarized by presenting the number and percentage of subjects having any ocular AE. Any other information collected (such as severity or relationship to study drug and intracameral injection) were listed as appropriate.

Analysis of the Primary Efficacy Variable: A full Statistical Analysis Plan was created prior to database lock. All post-hoc analyses were identified and described in the Clinical Study Report. When possible, statistical testing was applied.

Analysis Populations and Sets: The analysis sets for this clinical study are defined in accordance with ICH E9 Guidance for Industry Statistical Principles for Clinical Trials. The Full Analysis Set was intended to be the population which is as complete as possible and as close as possible to the intention-to-treat ideal. This was an open label study and not randomized, therefore the Full Analysis Set included only subjects who had received any study treatment and was the same as the Safety Population. Analysis sets are described below in Table 12.

TABLE 12
Analysis Sets

| Analysis Set | Description of Population |
|---|---|
| Full | Received any study treatment (at least |
| Analysis Set* | 1 complete or partial dose) |
| Per | Met all eligibility criteria |
| Protocol Set | Have received all study treatments per protocol |
| | Underwent DWEK/DSO of 4 to 5 mm |
| | Have sufficient data collected for the accurate |
| | assessment of study endpoints |

*The Full Analysis Set will be used for the Safety Population

The Per Protocol Population was used to assess the primary and secondary efficacy endpoints compared to the control group.

The Safety Population set included all participants in participants who received any TTHX1114 and was the population for safety outcomes; this population was compared with the control group. Ocular AEs involving the study eye were tabulated. Ocular AEs for the fellow eye and non-ocular AEs were provided as a listing.

### Clinical Results

The Positive Predictive Value of First Eye Recovery in Patients Undergoing Descemet Stripping Only (DSO) with TTHX1114

Purpose: Fuchs Endothelial Corneal Dystrophy (FECD) is a genetic condition and thereby has bilateral implications. DSO is an important surgical intervention for some patients with FECD; as DSO is further investigated as a effective intervention one must consider the potential confounding contribution of including second eyes in efficacy analyses. We reviewed the data from the STORM Study (TTHX-002) to compare outcomes in patients who underwent bilateral DSO with TTHX1114.

Methods: Study TTHX-002 was an open-label study of TTHX1114 (an engineered FGF1) administered as an adjunct to DSO that included 3 active regimens. Subjects were assigned to treatment at the discretion of the Investigator. There was a dose response noted between the low-dose and the 2 high-dose groups. Efficacy was similar in the 2 high-dose groups, 49 patients were enrolled; 45 of which were evaluable in the Per Protocol Population, 13 of which underwent bilateral DSO with TTHX1114. Not all subjects were eligible for bilateral procedures. The primary efficacy variable was Best-corrected Visual Acuity (BCVA) measured using the Early Treatment Diabetic Retinopathy Scale (ETDRS) scoring system where ≥69 letters=≥20/40 BCVA.

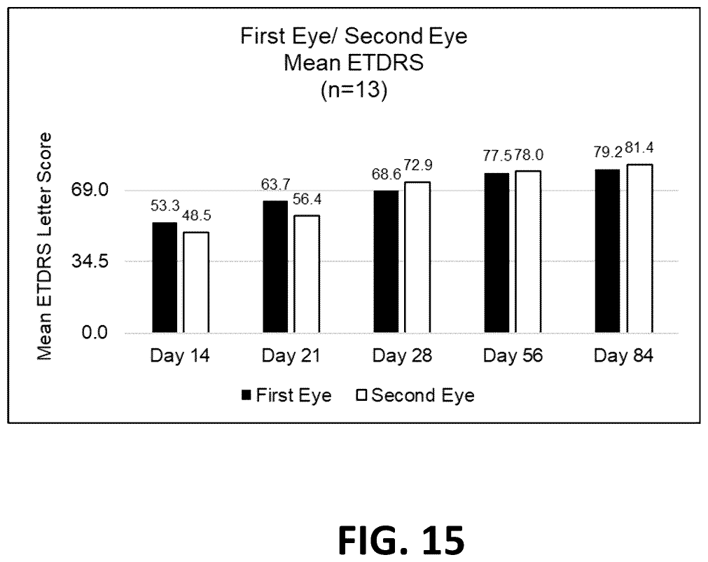

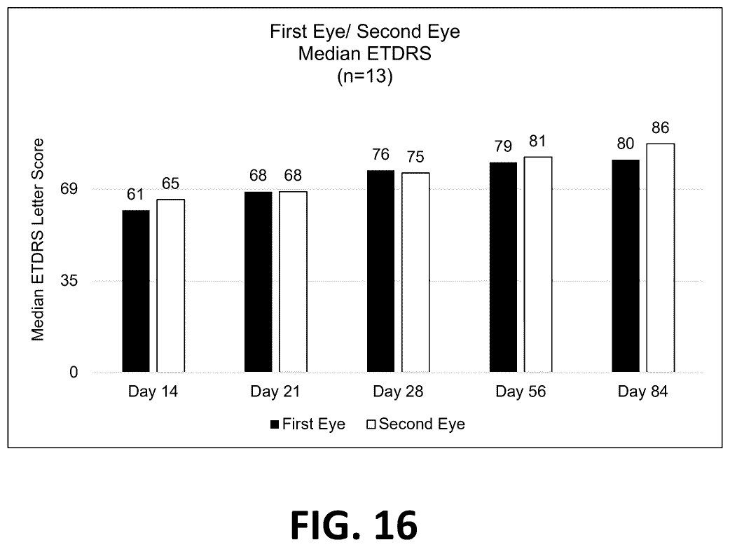

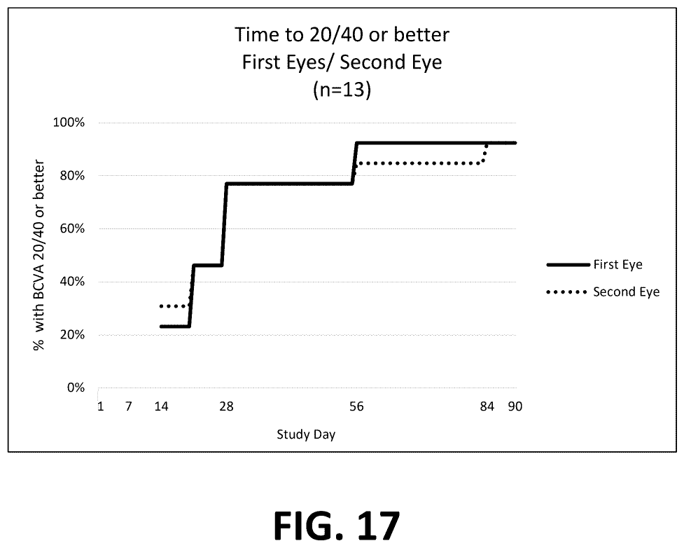

Results: The mean and median ETDRS Letter Score was similar for both sets of eyes at all timepoints and the overall responder rate was similar as well as shown FIGS. 15-17.

It is important to note that the 1 subject who had not responded to an ETDRS Letter Score of ≥69 letters was the same subject in the First Eye and Second Eye Groups; this subject's first eye had recovered to 78 letters at Day 168 and second eye had recovered to 80 letters at the Day 168 visit.

Conclusions: Of the 13 subjects who elected to have second eye DSO with TTHX1114, all had had positive results from the first eye procedure. Data related to the decision to proceed (or not) with second eye surgery were not collected. The potential negative predictive value of a poor outcome from the first eye surgery cannot be determined from this dataset.

Factors Affecting Recovery Following Descemet Stripping Only (DSO) with TTHX1114

Purpose: Recovery following DSO can be affected by many variables, including, but not limited to, size of descemetorhexis, comorbid diabetes, and concurrent cataract procedure. We reviewed the data from the STORM Study (TTHX-002) to determine the effect of these variables on time to recovery to BCVA of 20/40 or better.

Methods: Study TTHX-002 was an open-label study of TTHX1114 (an engineered FGF1) administered as an adjunct to DSO in FECD patients that demonstrated a dose-dependent acceleration of recovery based on both BCVA and corneal edema. Since efficacy was similar in the 2 high-dose groups (n=50, per protocol) these data were pooled and recovery rates in subset populations were analyzed. 45/50 (90%) of eyes had recovered to BCVA 20/40 or better at the time of data cut-off.

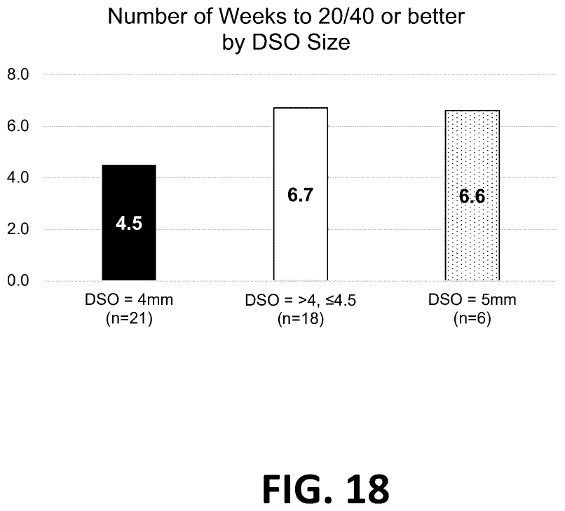

Results: Time to 20/40 (weeks) was faster in subjects with smaller DSO (4 mm=4.5, >4.5 to ≤4.5 mm=6.7, ≥5 mm=6.6) and slower in subjects with diabetes (7.7 compared to 5.2 in subjects without diabetes). These trends in faster recover were also seen at Day 28 where corneal edema was lower in subjects with smaller DSO (4 mm=637.6, >4.5 to ≤4.5 mm=743.2, ≥5 mm=808.8) and corneal thickness was slightly higher in in subjects with diabetes (719.8 μm compared to 702.9 in subjects without diabetes) as shown in FIGS. 18 & 19.

No significant differences were noticed in the recovery of subjects undergoing combination cataract surgery compared to those undergoing DSO alone. This suggests that TTHX1114 protects against the added damage of cataract surgery.

Conclusions: Smaller descemetorhexis was associated with faster recovery while comorbid diabetes appeared to delay recovery. Visual outcomes for patients undergoing DSO did not appear to be adversely affected by concurrent cataract surgery. Although these numbers are small, comorbidity and planned combination procedures should be taken into consideration when designing trials in DSO.

### Example 2: Protection of Endothelial Cells by TTHX1114 in Patients with Fuchs Endothelial Corneal Dystrophy (FECD) Undergoing Descemet's Stripping Only (DSO) in Combination with Cataract Surgery

Purpose: TTHX1114 is an FGF1 analog with protective, proliferative and pro-migratory properties for corneal endothelial cells (CEnCs). Cataract surgery is known to damage CEnCs. This study sought to analyze data from the STORM study to evaluate whether TTHX1114 protected endothelial cells from damage due to cataract surgery in FECD patients undergoing DSO.

Methods: This clinical study was an open-label, nonrandomized, Phase 2 dose ranging study of TTHX1114 in FECD patients undergoing DSO. In STORM, 46% of eyes underwent cataract surgery in combination with DSO. Subgroup analysis was used to determine if the group that received combined cataract surgery had more severe edema, worse BCVA or any other signs that the cataract surgery had the expected effect of endothelial damage.

Results: Across all patients, TTHX1114 produced a dose-dependent improvement in corneal edema and BCVA following surgery. The proportion of patients with complete resolution of corneal edema in the high dose (100 ng at time of surgery with or without additional post-surgical doses) groups at Day 28 was similar in patients with DSO only vs those with DSO+cataract (25.9% vs 17.4%) as was the mean central corneal thickness (705+/−174 um vs 700+/−148 um). The proportion of patients recovering good BCVA (69 letters or better) was also similar between these groups, with 58% of the DSO only patients recovering to 69 letters or better at day 28 vs 61% of the DSO+cataract patients. In the low dose (50 ng at time of surgery) group, central corneal thickness at 14 days was numerically worse in the DSO+cataract group (961+/−77) than the DSO only group (828+/−108 um) but this was not statistically significant, and the two groups are comparable at all later time points.

Conclusions: Cataract surgery when combined with DSO did not worsen postsurgical edema or adversely impact BCVA recovery. These data support the potential use of TTHX1114 for the control of postsurgical edema in high risk patients undergoing cataract surgery.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES

| SEQUENCE | No. |
|---|---|
| FNLPPGNYKKPKLLYCSNGGHFLRILPDGTVDGTRDRSDQHIQLQLSAESVGEVY | 1 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MFNLPPGNYKKPKLLYSSNGGHFLRILPDGTVDGTRDRSDQHIQLQLSAESVGEVYIKSTETGQYLCMD | 2 |
| TDGLLYGSQTPNEECLFLERLEENHYNTYISKKHAEKNWFVGLKKNGSVKRGPRTHYGQKAILFLPLPV | |
| SSD | |
| MAEGEITTFTALTEK | 3 |
| ALTEK | 4 |
| LTEK | 5 |
| TEK | 6 |
| EK | 7 |
| K | 8 |
| MALTEKFNLPPGNYKKPKLLYCSNGGHFLRILPDGTVDGTRDRSDQHIQLQLSAESVGEVY | 9 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKFNLPPGNYKKPKLLYCSNGGHFLRILPDGTVDGTRDRSDQHIQLQLSAESVGEVY | 10 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKFNLPPGNYKKPKLLYCSNGGHFLRILPDGTVDGTRDRSDQHIQLQLSAESVGEVY | 11 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKFNLPPGNYKKPKLLYCSNGGHFLRILPDGTVDGTRDRSDQHIQLQLSAESVGEVY | 12 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKFNLPPGNYKKPKLLYCSNGGHFLRILPDGTVDGTRDRSDQHIQLQLSAESVGEVY | 13 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MALTEKFNLPPGNYKKPKLLYSSNGGHFLRILPDGTVDGTRDRSDQHIQLQLSAESVGEVYIKSTETGQ | 14 |
| YLCMDTDGLLYGSQTPNEECLELERLEENHYNTYISKKHAEKNWFVGLKKNGSVKRGPRTHYGQKAILF | |
| LPLPVSSD | |
| MLTEKFNLPPGNYKKPKLLYSSNGGHFLRILPDGTVDGTRDRSDQHIQLQLSAESVGEVYIKSTETGQY | 15 |
| LCMDTDGLLYGSQTPNEECLFLERLEENHYNTYISKKHAEKNWFVGLKKNGSVKRGPRTHYGQKAILFL | |
| PLPVSSD | |
| MTEKFNLPPGNYKKPKLLYSSNGGHFLRILPDGTVDGTRDRSDQHIQLQLSAESVGEVYIKSTETGQYL | 16 |
| CMDTDGLLYGSQTPNEECLFLERLEENHYNTYISKKHAEKNWFVGLKKNGSVKRGPRTHYGQKAILFLP | |
| LPVSSD | |
| MEKFNLPPGNYKKPKLLYSSNGGHFLRILPDGTVDGTRDRSDQHIQLQLSAESVGEVYIKSTETGQYLC | 17 |
| MDTDGLLYGSQTPNEECLFLERLEENHYNTYISKKHAEKNWFVGLKKNGSVKRGPRTHYGQKAILFLPL | |
| PVSSD | |
| MKFNLPPGNYKKPKLLYSSNGGHFLRILPDGTVDGTRDRSDQHIQLQLSAESVGEVYIKSTETGQYLCM | 18 |
| DTDGLLYGSQTPNEECLFLERLEENHYNTYISKKHAEKNWFVGLKKNGSVKRGPRTHYGQKAILFLPLP | |
| VSSD | |
| ALTEKFNLPPGNYKKPKLLYCSNGGHFLRILPDGTVDGTRDRSDQHIQLQLSAESVGEVY | 19 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKFNLPPGNYKKPKLLYCSNGGHFLRILPDGTVDGTRDRSDQHIQLQLSAESVGEVY | 20 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKFNLPPGNYKKPKLLYCSNGGHFLRILPDGTVDGTRDRSDQHIQLQLSAESVGEVY | 21 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKFNLPPGNYKKPKLLYCSNGGHFLRILPDGTVDGTRDRSDQHIQLQLSAESVGEVY | 22 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KFNLPPGNYKKPKLLYCSNGGHFLRILPDGTVDGTRDRSDQHIQLQLSAESVGEVY | 23 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKFNLPPGNYKKPKLLYSSNGGHFLRILPDGTVDGTRDRSDQHIQLQLSAESVGEVYIKSTETGQY | 24 |
| LCMDTDGLLYGSQTPNEECLFLERLEENHYNTYISKKHAEKNWFVGLKKNGSVKRGPRTHYGQKAILEL | |
| PLPVSSD | |
| LTEKFNLPPGNYKKPKLLYSSNGGHELRILPDGTVDGTRDRSDQHIQLQLSAESVGEVYIKSTETGQYL | 25 |
| CMDTDGLLYGSQTPNEECLFLERLEENHYNTYISKKHAEKNWFVGLKKNGSVKRGPRTHYGQKAILFLP | |
| LPVSSD | |
| TEKFNLPPGNYKKPKLLYSSNGGHFLRILPDGTVDGTRDRSDQHIQLQLSAESVGEVYIKSTETGQYLC | 26 |
| MDTDGLLYGSQTPNEECLFLERLEENHYNTYISKKHAEKNWFVGLKKNGSVKRGPRTHYGQKAILFLPL | |
| PVSSD | |
| EKFNLPPGNYKKPKLLYSSNGGHFLRILPDGTVDGTRDRSDQHIQLQLSAESVGEVYIKSTETGQYLCM | 27 |
| DTDGLLYGSQTPNEECLFLERLEENHYNTYISKKHAEKNWFVGLKKNGSVKRGPRTHYGQKAILFLPLP | |
| VSSD | |
| KFNLPPGNYKKPKLLYSSNGGHFLRILPDGTVDGTRDRSDQHIQLQLSAESVGEVYIKSTETGQYLCMD | 28 |
| TDGLLYGSQTPNEECLFLERLEENHYNTYISKKHAEKNWFVGLKKNGSVKRGPRTHYGQKAILFLPLPV | |
| SSD | |
| NLPPGNYKKPKLLYCSNGGHFLRILPDGTVDGTRDRSDQHIQLQLSAESVGEVY | 29 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LPPGNYKKPKLLYCSNGGHFLRILPDGTVDGTRDRSDQHIQLQLSAESVGEVY | 30 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| PPGNYKKPKLLYCSNGGHFLRILPDGTVDGTRDRSDQHIQLQLSAESVGEVY | 31 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| PGNYKKPKLLYCSNGGHFLRILPDGTVDGTRDRSDQHIQLQLSAESVGEVY | 32 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| NLPPGNYKKPKLLYSSNGGHFLRILPDGTVDGTRDRSDQHIQLQLSAESVGEVYIKSTETGQYLCMDTD | 33 |
| GLLYGSQTPNEECLFLERLEENHYNTYISKKHAEKNWFVGLKKNGSVKRGPRTHYGQKAILELPLPVSS | |
| D | |
| LPPGNYKKPKLLYSSNGGHFLRILPDGTVDGTRDRSDQHIQLQLSAESVGEVYIKSTETGQYLCMDTDG | 34 |
| LLYGSQTPNEECLFLERLEENHYNTYISKKHAEKNWFVGLKKNGSVKRGPRTHYGQKAILFLPLPVSSD | |
| PPGNYKKPKLLYSSNGGHFLRILPDGTVDGTRDRSDQHIQLQLSAESVGEVYIKSTETGQYLCMDTDGL | 35 |
| LYGSQTPNEECLFLERLEENHYNTYISKKHAEKNWFVGLKKNGSVKRGPRTHYGQKAILFLPLPVSSD | |
| PGNYKKPKLLYSSNGGHFLRILPDGTVDGTRDRSDQHIQLQLSAESVGEVYIKSTETGQYLCMDTDGLL | 36 |
| YGSQTPNEECLFLERLEENHYNTYISKKHAEKNWFVGLKKNGSVKRGPRTHYGQKAILFLPLPVSSD | |
| MNLPPGNYKKPKLLYCSNGGHFLRILPDGTVDGTRDRSDQHIQLQLSAESVGEVY | 37 |
| IKSTETGQYLAMDTDGLLYGSQTPNEECLELERLEENHYNTYISKKHAEK | |
| NWFVGLKKNGSCKRGPRTHYGQKAILFLPLPVSSD | |
| MLPPGNYKKPKLLYCSNGGHFLRILPDGTVDGTRDRSDQHIQLQLSAESVGEVY | 38 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MPPGNYKKPKLLYCSNGGHFLRILPDGTVDGTRDRSDQHIQLQLSAESVGEVY | 39 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MPGNYKKPKLLYCSNGGHFLRILPDGTVDGTRDRSDQHIQLQLSAESVGEVY | 40 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MNLPPGNYKKPKLLYSSNGGHFLRILPDGTVDGTRDRSDQHIQLQLSAESVGEVYIKSTETGQYLCMDT | 41 |
| DGLLYGSQTPNEECLFLERLEENHYNTYISKKHAEKNWFVGLKKNGSVKRGPRTHYGQKAILELPLPVS | |
| SD | |
| MLPPGNYKKPKLLYSSNGGHFLRILPDGTVDGTRDRSDQHIQLQLSAESVGEVYIKSTETGQYLCMDTD | 42 |
| GLLYGSQTPNEECLFLERLEENHYNTYISKKHAEKNWFVGLKKNGSVKRGPRTHYGQKAILFLPLPVSS | |
| D | |
| MPPGNYKKPKLLYSSNGGHFLRILPDGTVDGTRDRSDQHIQLQLSAESVGEVYIKSTETGQYLCMDTDG | 43 |
| LLYGSQTPNEECLFLERLEENHYNTYISKKHAEKNWFVGLKKNGSVKRGPRTHYGQKAILFLPLPVSSD | |
| MPGNYKKPKLLYSSNGGHFLRILPDGTVDGTRDRSDQHIQLQLSAESVGEVYIKSTETGQYLCMDTDGL | 44 |
| LYGSQTPNEECLFLERLEENHYNTYISKKHAEKNWFVGLKKNGSVKRGPRTHYGQKAILFLPLPVSSD | |
| MALTEKNLPPGNYKKPKLLYCSNGGHFLRILPDGTVDGTRDRSDQHIQLQLSAESVGEVY | 45 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MALTEKLPPGNYKKPKLLYSSNGGHFLRILPDGTVDGTRDRSDQHIQLQLSAESVGEVYIKSTETGQYL | 46 |
| CMDTDGLLYGSQTPNEECLFLERLEENHYNTYISKKHAEKNWFVGLKKNGSVKRGPRTHYGQKAILFLP | |
| LPVSSD | |
| MALTEKPPGGNYKKPKLLYCSNGGHFLRILPDGTVDGTRDRSDQHIQLQLSAESVGEVY | 47 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MALTEKPGNYKKPKLLYCSNGGHFLRILPDGTVDGTRDRSDQHIQLQLSAESVGEVY | 48 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKFNLPPGNYKKPKLLYCSNGGHFLRILPDGTVDGTRDRSDQHIQLQLSAESVGEVY | 49 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKNLPPGNYKKPKLLYCSNGGHFLRILPDGTVDGTRDRSDQHIQLQLSAESVGEVY | 50 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKLPPGNYKKPKLLYCSNGGHFLRILPDGTVDGTRDRSDQHIQLQLSAESVGEVY | 51 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKPPGNYKKPKLLYCSNGGHFLRILPDGTVDGTRDRSDQHIQLQLSAESVGEVY | 52 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MLTEKPGNYKKPKLLYCSNGGHFLRILPDGTVDGTRDRSDQHIQLQLSAESVGEVY | 53 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKFNLPPGNYKKPKLLYCSNGGHFLRILPDGTVDGTRDRSDQHIQLQLSAESVGEVY | 54 |
| IKSTETGQYLAMDTDGLLYGSQTPNEECLELERLEENHYNTYISKKHAEK | |
| NWFVGLKKNGSCKRGPRTHYGQKAILELPLPVSSD | |
| MTEKNLPPGNYKKPKLLYCSNGGHFLRILPDGTVDGTRDRSDQHIQLQLSAESVGEVY | 55 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKLPPGNYKKPKLLYCSNGGHFLRILPDGTVDGTRDRSDQHIQLQLSAESVGEVY | 56 |
| IKSTETGQYLAMDTDGLLYGSQTPNEECLFLERLEENHYNTYISKKHAEK | |
| NWFVGLKKNGSCKRGPRTHYGQKAILELPLPVSSD | |
| MTEKPPGNYKKPKLLYCSNGGHFLRILPDGTVDGTRDRSDQHIQLQLSAESVGEVY | 57 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MTEKPGNYKKPKLLYCSNGGHFLRILPDGTVDGTRDRSDQHIQLQLSAESVGEVY | 58 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKFNLPPGNYKKPKLLYCSNGGHFLRILPDGTVDGTRDRSDQHIQLQLSAESVGEVY | 59 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKNLPPGNYKKPKLLYCSNGGHFLRILPDGTVDGTRDRSDQHIQLQLSAESVGEVY | 60 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKLPPGNYKKPKLLYCSNGGHFLRILPDGTVDGTRDRSDQHIQLQLSAESVGEVY | 61 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKPPGNYKKPKLLYCSNGGHFLRILPDGTVDGTRDRSDQHIQLQLSAESVGEVY | 62 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MEKPGNYKKPKLLYCSNGGHFLRILPDGTVDGTRDRSDQHIQLQLSAESVGEVY | 63 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKFNLPPGNYKKPKLLYCSNGGHFLRILPDGTVDGTRDRSDQHIQLQLSAESVGEVY | 64 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKNLPPGNYKKPKLLYCSNGGHFLRILPDGTVDGTRDRSDQHIQLQLSAESVGEVY | 65 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKLPPGNYKKPKLLYCSNGGHFLRILPDGTVDGTRDRSDQHIQLQLSAESVGEVY | 66 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKPPGNYKKPKLLYCSNGGHFLRILPDGTVDGTRDRSDQHIQLQLSAESVGEVY | 67 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| MKPGNYKKPKLLYCSNGGHFLRILPDGTVDGTRDRSDQHIQLQLSAESVGEVY | 68 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKNLPPGNYKKPKLLYCSNGGHFLRILPDGTVDGTRDRSDQHIQLQLSAESVGEVY | 69 |
| IKSTETGQYLAMDTDGLLYGSQTPNEECLFLERLEENHYNTYISKKHAEK | |
| NWFVGLKKNGSCKRGPRTHYGQKAILELPLPVSSD | |
| ALTEKLPPGNYKKPKLLYCSNGGHFLRILPDGTVDGTRDRSDQHIQLQLSAESVGEVY | 70 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKPPGNYKKPKLLYCSNGGHFLRILPDGTVDGTRDRSDQHIQLQLSAESVGEVY | 71 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKPGNYKKPKLLYCSNGGHFLRILPDGTVDGTRDRSDQHIQLQLSAESVGEVY | 72 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKFNLPPGNYKKPKLLYCSNGGHFLRILPDGTVDGTRDRSDQHIQLQLSAESVGEVY | 73 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKNLPPGNYKKPKLLYCSNGGHFLRILPDGTVDGTRDRSDQHIQLQLSAESVGEVY | 74 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKLPPGNYKKPKLLYCSNGGHFLRILPDGTVDGTRDRSDQHIQLQLSAESVGEVY | 75 |
| IKSTETGQYLAMDTDGLLYGSQTPNEECLFLERLEENHYNTYISKKHAEK | |
| NWFVGLKKNGSCKRGPRTHYGQKAILELPLPVSSD | |
| LTEKPPGNYKKPKLLYCSNGGHFLRILPDGTVDGTRDRSDQHIQLQLSAESVGEVY | 76 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| LTEKPGNYKKPKLLYCSNGGHFLRILPDGTVDGTRDRSDQHIQLQLSAESVGEVY | 77 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKFNLPPGNYKKPKLLYCSNGGHFLRILPDGTVDGTRDRSDQHIQLQLSAESVGEVY | 78 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKNLPPGNYKKPKLLYCSNGGHFLRILPDGTVDGTRDRSDQHIQLQLSAESVGEVY | 79 |
| IKSTETGQYLAMDTDGLLYGSQTPNEECLELERLEENHYNTYISKKHAEK | |
| NWFVGLKKNGSCKRGPRTHYGQKAILFLPLPVSSD | |
| TEKLPPGNYKKPKLLYCSNGGHFLRILPDGTVDGTRDRSDQHIQLQLSAESVGEVY | 80 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKPPGNYKKPKLLYCSNGGHFLRILPDGTVDGTRDRSDQHIQLQLSAESVGEVY | 81 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| TEKPGNYKKPKLLYCSNGGHFLRILPDGTVDGTRDRSDQHIQLQLSAESVGEVY | 82 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKFNLPPGNYKKPKLLYCSNGGHFLRILPDGTVDGTRDRSDQHIQLQLSAESVGEVY | 83 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKNLPPGNYKKPKLLYCSNGGHFLRILPDGTVDGTRDRSDQHIQLQLSAESVGEVY | 84 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKLPPGNYKKPKLLYCSNGGHFLRILPDGTVDGTRDRSDQHIQLQLSAESVGEVY | 85 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKPPGNYKKPKLLYCSNGGHFLRILPDGTVDGTRDRSDQHIQLQLSAESVGEVY | 86 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| EKPGNYKKPKLLYCSNGGHFLRILPDGTVDGTRDRSDQHIQLQLSAESVGEVY | 87 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KFNLPPGNYKKPKLLYCSNGGHFLRILPDGTVDGTRDRSDQHIQLQLSAESVGEVY | 88 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KNLPPGNYKKPKLLYCSNGGHFLRILPDGTVDGTRDRSDQHIQLQLSAESVGEVY | 89 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KLPPGNYKKPKLLYCSNGGHELRILPDGTVDGTRDRSDQHIQLQLSAESVGEVY | 90 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| KPPGNYKKPKLLYCSNGGHELRILPDGTVDGTRDRSDQHIQLQLSAESVGEVY | 91 |
| IKSTETGQYLAMDTDGLLYGSQTPNEECLFLERLEENHYNTYISKKHAEK | |
| NWFVGLKKNGSCKRGPRTHYGQKAILELPLPVSSD | |
| KPGNYKKPKLLYCSNGGHFLRILPDGTVDGTRDRSDQHIQLQLSAESVGEVY | 92 |
| IKSTETGQYLAMDTDGLLYGSQTPNEECLFLERLEENHYNTYISKKHAEK | |
| NWFVGLKKNGSCKRGPRTHYGQKAILFLPLPVSSD | |
| ALTEKNLPPGNYKKPKLLYSSNGGHFLRILPDGTVDGTRDRSDQHIQLQLSAESVGEVYIKSTETGQYL | 93 |
| CMDTDGLLYGSQTPNEECLFLERLEENHYNTYISKKHAEKNWFVGLKKNGSVKRGPRTHYGQKAILFLP | |
| LPVSSD | |
| ALTEKLPPGNYKKPKLLYSSNGGHELRILPDGTVDGTRDRSDQHIQLQLSAESVGEVYIKSTETGQYLC | 94 |
| MDTDGLLYGSQTPNEECLFLERLEENHYNTYISKKHAEKNWFVGLKKNGSVKRGPRTHYGQKAILFLPL | |
| PVSSD | |
| ALTEKPPGNYKKPKLLYSSNGGHFLRILPDGTVDGTRDRSDQHIQLQLSAESVGEVYIKSTETGQYLCM | 96 |
| DTDGLLYGSQTPNEECLFLERLEENHYNTYISKKHAEKNWFVGLKKNGSVKRGPRTHYGQKAILELPLP | |
| VSSD | |
| ALTEKPGNYKKPKLLYSSNGGHFLRILPDGTVDGTRDRSDQHIQLQLSAESVGEVYIKSTETGQYLCMD | 97 |
| TDGLLYGSQTPNEECLFLERLEENHYNTYISKKHAEKNWFVGLKKNGSVKRGPRTHYGQKAILFLPLPV | |
| SSD | |
| LTEKFNLPPGNYKKPKLLYSSNGGHFLRILPDGTVDGTRDRSDQHIQLQLSAESVGEVYIKSTETGQYL | 98 |
| CMDTDGLLYGSQTPNEECLELERLEENHYNTYISKKHAEKNWFVGLKKNGSVKRGPRTHYGQKAILFLP | |
| LPVSSD | |
| LTEKNLPPGNYKKPKLLYSSNGGHFLRILPDGTVDGTRDRSDQHIQLQLSAESVGEVYIKSTETGQYLC | 99 |
| MDTDGLLYGSQTPNEECLFLERLEENHYNTYISKKHAEKNWFVGLKKNGSVKRGPRTHYGQKAILFLPL | |
| PVSSD | |
| LTEKLPPGNYKKPKLLYSSNGGHFLRILPDGTVDGTRDRSDQHIQLQLSAESVGEVYIKSTETGQYLCM | 100 |
| DTDGLLYGSQTPNEECLELERLEENHYNTYISKKHAEKNWFVGLKKNGSVKRGPRTHYGQKAILFLPLP | |
| VSSD | |
| LTEKPPGNYKKPKLLYSSNGGHFLRILPDGTVDGTRDRSDQHIQLQLSAESVGEVYIKSTETGQYLCMD | 101 |
| TDGLLYGSQTPNEECLFLERLEENHYNTYISKKHAEKNWFVGLKKNGSVKRGPRTHYGQKAILFLPLPV | |
| SSD | |
| LTEKPGNYKKPKLLYSSNGGHFLRILPDGTVDGTRDRSDQHIQLQLSAESVGEVYIKSTETGQYLCMDT | 102 |
| DGLLYGSQTPNEECLELERLEENHYNTYISKKHAEKNWFVGLKKNGSVKRGPRTHYGQKAILFLPLPVS | |
| SD | |
| TEKFNLPPGNYKKPKLLYSSNGGHELRILPDGTVDGTRDRSDQHIQLQLSAESVGEVYIKSTETGQYLC | 103 |
| MDTDGLLYGSQTPNEECLELERLEENHYNTYISKKHAEKNWFVGLKKNGSVKRGPRTHYGQKAILFLPL | |
| PVSSD | |
| TEKNLPPGNYKKPKLLYSSNGGHFLRILPDGTVDGTRDRSDQHIQLQLSAESVGEVYIKSTETGQYLCM | 104 |
| DTDGLLYGSQTPNEECLELERLEENHYNTYISKKHAEKNWFVGLKKNGSVKRGPRTHYGQKAILELPLP | |
| VSSD | |
| TEKLPPGNYKKPKLLYSSNGGHFLRILPDGTVDGTRDRSDQHIQLQLSAESVGEVYIKSTETGQYLCMD | 105 |
| TDGLLYGSQTPNEECLFLERLEENHYNTYISKKHAEKNWFVGLKKNGSVKRGPRTHYGQKAILFLPLPV | |
| SSD | |
| TEKPPGNYKKPKLLYSSNGGHFLRILPDGTVDGTRDRSDQHIQLQLSAESVGEVYIKSTETGQYLCMDT | 106 |
| DGLLYGSQTPNEECLFLERLEENHYNTYISKKHAEKNWFVGLKKNGSVKRGPRTHYGQKAILFLPLPVS | |
| SD | |
| TEKPGNYKKPKLLYSSNGGHFLRILPDGTVDGTRDRSDQHIQLQLSAESVGEVYIKSTETGQYLCMDTD | 107 |
| GLLYGSQTPNEECLFLERLEENHYNTYISKKHAEKNWFVGLKKNGSVKRGPRTHYGQKAILELPLPVSS | |
| D | |
| EKFNLPPGNYKKPKLLYSSNGGHELRILPDGTVDGTRDRSDQHIQLQLSAESVGEVYIKSTETGQYLCM | 108 |
| DTDGLLYGSQTPNEECLFLERLEENHYNTYISKKHAEKNWFVGLKKNGSVKRGPRTHYGQKAILFLPLP | |
| VSSD | |
| EKNLPPGNYKKPKLLYSSNGGHFLRILPDGTVDGTRDRSDQHIQLQLSAESVGEVYIKSTETGQYLCMD | 109 |
| TDGLLYGSQTPNEECLFLERLEENHYNTYISKKHAEKNWFVGLKKNGSVKRGPRTHYGQKAILFLPLPV | |
| SSD | |
| EKLPPGNYKKPKLLYSSNGGHFLRILPDGTVDGTRDRSDQHIQLQLSAESVGEVYIKSTETGQYLCMDT | 110 |
| DGLLYGSQTPNEECLFLERLEENHYNTYISKKHAEKNWFVGLKKNGSVKRGPRTHYGQKAILFLPLPVS | |
| SD | |
| EKPPGNYKKPKLLYSSNGGHELRILPDGTVDGTRDRSDQHIQLQLSAESVGEVYIKSTETGQYLCMDTD | 111 |
| GLLYGSQTPNEECLFLERLEENHYNTYISKKHAEKNWFVGLKKNGSVKRGPRTHYGQKAILFLPLPVSS | |
| D | |
| EKPGNYKKPKLLYSSNGGHFLRILPDGTVDGTRDRSDQHIQLQLSAESVGEVYIKSTETGQYLCMDTDG | 112 |
| LLYGSQTPNEECLFLERLEENHYNTYISKKHAEKNWFVGLKKNGSVKRGPRTHYGQKAILFLPLPVSSD | |
| KFNLPPGNYKKPKLLYSSNGGHFLRILPDGTVDGTRDRSDQHIQLQLSAESVGEVYIKSTETGQYLCMD | 113 |
| TDGLLYGSQTPNEECLELERLEENHYNTYISKKHAEKNWFVGLKKNGSVKRGPRTHYGQKAILFLPLPV | |
| SSD | |
| KNLPPGNYKKPKLLYSSNGGHFLRILPDGTVDGTRDRSDQHIQLQLSAESVGEVYIKSTETGQYLCMDT | 114 |
| DGLLYGSQTPNEECLFLERLEENHYNTYISKKHAEKNWFVGLKKNGSVKRGPRTHYGQKAILELPLPVS | |
| SD | |
| KLPPGNYKKPKLLYSSNGGHFLRILPDGTVDGTRDRSDQHIQLQLSAESVGEVYIKSTETGQYLCMDTD | 115 |
| GLLYGSQTPNEECLFLERLEENHYNTYISKKHAEKNWFVGLKKNGSVKRGPRTHYGQKAILELPLPVSS | |
| D | |
| KPPGNYKKPKLLYSSNGGHFLRILPDGTVDGTRDRSDQHIQLQLSAESVGEVYIKSTETGQYLCMDTDG | 116 |
| LLYGSQTPNEECLFLERLEENHYNTYISKKHAEKNWFVGLKKNGSVKRGPRTHYGQKAILELPLPVSSD | |
| KPGNYKKPKLLYSSNGGHFLRILPDGTVDGTRDRSDQHIQLQLSAESVGEVYIKSTETGQYLCMDTDGL | 117 |
| LYGSQTPNEECLFLERLEENHYNTYISKKHAEKNWFVGLKKNGSVKRGPRTHYGQKAILFLPLPVSSD | |
| MALTEKFNLPPGNYKKPKLLYSSNGGHFLRILPDGTVDGTRDRSDQHIQLQLSAESVGEVYIKSTETGQ | 118 |
| YLCMDTDGLLYGSQTPNEECLFLERLEENHYNTYISKKHAEKNWFVGLKKNGSVKRGPRTHYGQKAILE | |
| LPLPVSSD | |
| MALTEKNLPPGNYKKPKLLYSSNGGHELRILPDGTVDGTRDRSDQHIQLQLSAESVGEVYIKSTETGQY | 207 |
| LCMDTDGLLYGSQTPNEECLFLERLEENHYNTYISKKHAEKNWFVGLKKNGSVKRGPRTHYGQKAILFL | |
| PLPVSSD | |
| MALTEKLPPGNYKKPKLLYSSNGGHFLRILPDGTVDGTRDRSDQHIQLQLSAESVGEVYIKSTETGQYL | 119 |
| CMDTDGLLYGSQTPNEECLFLERLEENHYNTYISKKHAEKNWFVGLKKNGSVKRGPRTHYGQKAILFLP | |
| LPVSSD | |
| MALTEKPPGNYKKPKLLYSSNGGHELRILPDGTVDGTRDRSDQHIQLQLSAESVGEVYIKSTETGQYLC | 120 |
| MDTDGLLYGSQTPNEECLELERLEENHYNTYISKKHAEKNWFVGLKKNGSVKRGPRTHYGQKAILFLPL | |
| PVSSD | |
| MALTEKPGNYKKPKLLYSSNGGHFLRILPDGTVDGTRDRSDQHIQLQLSAESVGEVYIKSTETGQYLCM | 121 |
| DTDGLLYGSQTPNEECLFLERLEENHYNTYISKKHAEKNWFVGLKKNGSVKRGPRTHYGQKAILFLPLP | |
| VSSD | |
| MLTEKFNLPPGNYKKPKLLYSSNGGHELRILPDGTVDGTRDRSDQHIQLQLSAESVGEVYIKSTETGQY | 122 |
| LCMDTDGLLYGSQTPNEECLFLERLEENHYNTYISKKHAEKNWFVGLKKNGSVKRGPRTHYGQKAILFL | |
| PLPVSSD | |
| MLTEKNLPPGNYKKPKLLYSSNGGHFLRILPDGTVDGTRDRSDQHIQLQLSAESVGEVYIKSTETGQYL | 123 |
| CMDTDGLLYGSQTPNEECLFLERLEENHYNTYISKKHAEKNWFVGLKKNGSVKRGPRTHYGQKAILFLP | |
| LPVSSD | |
| MLTEKLPPGNYKKPKLLYSSNGGHFLRILPDGTVDGTRDRSDQHIQLQLSAESVGEVYIKSTETGQYLC | 124 |
| MDTDGLLYGSQTPNEECLFLERLEENHYNTYISKKHAEKNWFVGLKKNGSVKRGPRTHYGQKAILFLPL | |
| PVSSD | |
| MLTEKPPGNYKKPKLLYSSNGGHFLRILPDGTVDGTRDRSDQHIQLQLSAESVGEVYIKSTETGQYLCM | 125 |
| DTDGLLYGSQTPNEECLFLERLEENHYNTYISKKHAEKNWFVGLKKNGSVKRGPRTHYGQKAILFLPLP | |
| VSSD | |
| MLTEKPGNYKKPKLLYSSNGGHFLRILPDGTVDGTRDRSDQHIQLQLSAESVGEVYIKSTETGQYLCMD | 126 |
| TDGLLYGSQTPNEECLFLERLEENHYNTYISKKHAEKNWFVGLKKNGSVKRGPRTHYGQKAILFLPLPV | |
| SSD | |
| MTEKFNLPPGNYKKPKLLYSSNGGHFLRILPDGTVDGTRDRSDQHIQLQLSAESVGEVYIKSTETGQYL | 127 |
| CMDTDGLLYGSQTPNEECLFLERLEENHYNTYISKKHAEKNWFVGLKKNGSVKRGPRTHYGQKAILFLP | |
| LPVSSD | |
| MTEKNLPPGNYKKPKLLYSSNGGHFLRILPDGTVDGTRDRSDQHIQLQLSAESVGEVYIKSTETGQYLC | 128 |
| MDTDGLLYGSQTPNEECLFLERLEENHYNTYISKKHAEKNWFVGLKKNGSVKRGPRTHYGQKAILELPL | |
| PVSSD | |
| MTEKLPPGNYKKPKLLYSSNGGHFLRILPDGTVDGTRDRSDQHIQLQLSAESVGEVYIKSTETGQYLCM | 129 |
| DTDGLLYGSQTPNEECLELERLEENHYNTYISKKHAEKNWFVGLKKNGSVKRGPRTHYGQKAILFLPLP | |
| VSSD | |
| MTEKPPGNYKKPKLLYSSNGGHFLRILPDGTVDGTRDRSDQHIQLQLSAESVGEVYIKSTETGQYLCMD | 130 |
| TDGLLYGSQTPNEECLFLERLEENHYNTYISKKHAEKNWFVGLKKNGSVKRGPRTHYGQKAILFLPLPV | |
| SSD | |
| MTEKPGNYKKPKLLYSSNGGHFLRILPDGTVDGTRDRSDQHIQLQLSAESVGEVYIKSTETGQYLCMDT | 131 |
| DGLLYGSQTPNEECLFLERLEENHYNTYISKKHAEKNWFVGLKKNGSVKRGPRTHYGQKAILFLPLPVS | |
| SD | |
| MEKFNLPPGNYKKPKLLYSSNGGHELRILPDGTVDGTRDRSDQHIQLQLSAESVGEVYIKSTETGQYLC | 132 |
| MDTDGLLYGSQTPNEECLFLERLEENHYNTYISKKHAEKNWFVGLKKNGSVKRGPRTHYGQKAILFLPL | |
| PVSSD | |
| MEKNLPPGNYKKPKLLYSSNGGHFLRILPDGTVDGTRDRSDQHIQLQLSAESVGEVYIKSTETGQYLCM | 133 |
| DTDGLLYGSQTPNEECLFLERLEENHYNTYISKKHAEKNWFVGLKKNGSVKRGPRTHYGQKAILFLPLP | |
| VSSD | |
| MEKLPPGNYKKPKLLYSSNGGHFLRILPDGTVDGTRDRSDQHIQLQLSAESVGEVYIKSTETGQYLCMD | 134 |
| TDGLLYGSQTPNEECLFLERLEENHYNTYISKKHAEKNWFVGLKKNGSVKRGPRTHYGQKAILELPLPV | |
| SSD | |
| MEKPPGNYKKPKLLYSSNGGHFLRILPDGTVDGTRDRSDQHIQLQLSAESVGEVYIKSTETGQYLCMDT | 135 |
| DGLLYGSQTPNEECLFLERLEENHYNTYISKKHAEKNWFVGLKKNGSVKRGPRTHYGQKAILFLPLPVS | |
| SD | |
| MEKPGNYKKPKLLYSSNGGHFLRILPDGTVDGTRDRSDQHIQLQLSAESVGEVYIKSTETGQYLCMDTD | 136 |
| GLLYGSQTPNEECLFLERLEENHYNTYISKKHAEKNWFVGLKKNGSVKRGPRTHYGQKAILFLPLPVSS | |
| D | |
| MKFNLPPGNYKKPKLLYSSNGGHFLRILPDGTVDGTRDRSDQHIQLQLSAESVGEVYIKSTETGQYLCM | 137 |
| DTDGLLYGSQTPNEECLFLERLEENHYNTYISKKHAEKNWFVGLKKNGSVKRGPRTHYGQKAILELPLP | |
| VSSD | |
| MKNLPPGNYKKPKLLYSSNGGHELRILPDGTVDGTRDRSDQHIQLQLSAESVGEVYIKSTETGQYLCMD | 138 |
| TDGLLYGSQTPNEECLFLERLEENHYNTYISKKHAEKNWFVGLKKNGSVKRGPRTHYGQKAILFLPLPV | |
| SSD | |
| MKLPPGNYKKPKLLYSSNGGHFLRILPDGTVDGTRDRSDQHIQLQLSAESVGEVYIKSTETGQYLCMDT | 139 |
| DGLLYGSQTPNEECLELERLEENHYNTYISKKHAEKNWFVGLKKNGSVKRGPRTHYGQKAILFLPLPVS | |
| SD | |
| MKPPGNYKKPKLLYSSNGGHFLRILPDGTVDGTRDRSDQHIQLQLSAESVGEVYIKSTETGQYLCMDTD | 140 |
| GLLYGSQTPNEECLFLERLEENHYNTYISKKHAEKNWFVGLKKNGSVKRGPRTHYGQKAILFLPLPVSS | |
| D | |
| MKPGNYKKPKLLYSSNGGHFLRILPDGTVDGTRDRSDQHIQLQLSAESVGEVYIKSTETGQYLCMDTDG | 141 |
| LLYGSQTPNEECLFLERLEENHYNTYISKKHAEKNWFVGLKKNGSVKRGPRTHYGQKAILFLPLPVSSD | |
| FNLPPGNYKKPKLLYCSNGGHFLRILPDGTVDGTRDRSDQHIQLQLSAESVGEVY | 142 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| NLPPGNYKKPKLLYCSNGGHFLRILPDGTVDGTRDRSDQHIQLQLSAESVGEVY | 143 |
| IKSTETGQYLAXDTDGLLYGSQTPNEECLELERLEENHYNTYISKKHAEK | |
| NWFVGLKKNGSCKRGPRTHYGQKAILELPLPVSSD | |
| PPGNYKKPKLLYCSNGGHFLRILPDGTVDGTRDRSDQHIQLQLSAESVGEVY | 144 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| PGNYKKPKLLYCSNGGHFLRILPDGTVDGTRDRSDQHIQLQLSAESVGEVY | 145 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| FNLPPGNYKKPKLLYSSNGGHFLRILPDGTVDGTRDRSDQHIQLQLSAESVGEVYIKSTETGQYLCXDT | 146 |
| DGLLYGSQTPNEECLFLERLEENHYNTYISKKHAEKNWFVGLKKNGSVKRGPRTHYGQKAILFLPLPVS | |
| SD | |
| NLPPGNYKKPKLLYSSNGGHFLRILPDGTVDGTRDRSDQHIQLQLSAESVGEVYIKSTETGQYLCXDTD | 147 |
| GLLYGSQTPNEECLFLERLEENHYNTYISKKHAEKNWFVGLKKNGSVKRGPRTHYGQKAILFLPLPVSS | |
| D | |
| PPGNYKKPKLLYSSNGGHFLRILPDGTVDGTRDRSDQHIQLQLSAESVGEVYIKSTETGQYLCXDTDGL | 148 |
| LYGSQTPNEECLFLERLEENHYNTYISKKHAEKNWFVGLKKNGSVKRGPRTHYGQKAILFLPLPVSSD | |
| PGNYKKPKLLYSSNGGHFLRILPDGTVDGTRDRSDQHIQLQLSAESVGEVYIKSTETGQYLCXDTDGLL | 149 |
| YGSQTPNEECLFLERLEENHYNTYISKKHAEKNWFVGLKKNGSVKRGPRTHYGQKAILFLPLPVSSD | |
| ALTEKNLPPGNYKKPKLLYCSNGGHFLRILPDGTVDGTRDRSDQHIQLQLSAESVGEVY | 150 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| ALTEKLPPGNYKKPKLLYSSNGGHFLRILPDGTVDGTRDRSDQHIQLQLSAESVGEVYIKSTETGQYLC | 151 |
| XDTDGLLYGSQTPNEECLFLERLEENHYNTYISKKHAEKNWFVGLKKNGSVKRGPRTHYGQKAILFLPL | |
| PVSSD | |
| ALTEKPPGGNYKKPKLLYCSNGGHFLRILPDGTVDGTRDRSDQHIQLQLSAESVGEVY | 152 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| ALTEKPGNYKKPKLLYCSNGGHFLRILPDGTVDGTRDRSDQHIQLQLSAESVGEVY | 153 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKFNLPPGNYKKPKLLYCSNGGHFLRILPDGTVDGTRDRSDQHIQLQLSAESVGEVY | 154 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKNLPPGNYKKPKLLYCSNGGHFLRILPDGTVDGTRDRSDQHIQLQLSAESVGEVY | 155 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKLPPGNYKKPKLLYCSNGGHFLRILPDGTVDGTRDRSDQHIQLQLSAESVGEVY | 156 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKPPGNYKKPKLLYCSNGGHFLRILPDGTVDGTRDRSDQHIQLQLSAESVGEVY | 157 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| LTEKPGNYKKPKLLYCSNGGHFLRILPDGTVDGTRDRSDQHIQLQLSAESVGEVY | 158 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKFNLPPGNYKKPKLLYCSNGGHFLRILPDGTVDGTRDRSDQHIQLQLSAESVGEVY | 159 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKNLPPGNYKKPKLLYCSNGGHFLRILPDGTVDGTRDRSDQHIQLQLSAESVGEVY | 160 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKLPPGNYKKPKLLYCSNGGHFLRILPDGTVDGTRDRSDQHIQLQLSAESVGEVY | 161 |
| IKSTETGQYLAXDTDGLLYGSQTPNEECLELERLEENHYNTYISKKHAEK | |
| NWFVGLKKNGSCKRGPRTHYGQKAILFLPLPVSSD | |
| TEKPPGNYKKPKLLYCSNGGHFLRILPDGTVDGTRDRSDQHIQLQLSAESVGEVY | 162 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| TEKPGNYKKPKLLYCSNGGHFLRILPDGTVDGTRDRSDQHIQLQLSAESVGEVY | 163 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKFNLPPGNYKKPKLLYCSNGGHFLRILPDGTVDGTRDRSDQHIQLQLSAESVGEVY | 164 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKNLPPGNYKKPKLLYCSNGGHFLRILPDGTVDGTRDRSDQHIQLQLSAESVGEVY | 165 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKLPPGNYKKPKLLYCSNGGHFLRILPDGTVDGTRDRSDQHIQLQLSAESVGEVY | 166 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKPPGNYKKPKLLYCSNGGHFLRILPDGTVDGTRDRSDQHIQLQLSAESVGEVY | 167 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| EKPGNYKKPKLLYCSNGGHFLRILPDGTVDGTRDRSDQHIQLQLSAESVGEVY | 168 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KFNLPPGNYKKPKLLYCSNGGHFLRILPDGTVDGTRDRSDQHIQLQLSAESVGEVY | 169 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KNLPPGNYKKPKLLYCSNGGHFLRILPDGTVDGTRDRSDQHIQLQLSAESVGEVY | 170 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KLPPGNYKKPKLLYCSNGGHFLRILPDGTVDGTRDRSDQHIQLQLSAESVGEVY | 171 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| KPPGNYKKPKLLYCSNGGHFLRILPDGTVDGTRDRSDQHIQLQLSAESVGEVY | 172 |
| IKSTETGQYLAXDTDGLLYGSQTPNEECLELERLEENHYNTYISKKHAEK | |
| NWFVGLKKNGSCKRGPRTHYGQKAILFLPLPVSSD | |
| KPGNYKKPKLLYCSNGGHFLRILPDGTVDGTRDRSDQHIQLQLSAESVGEVY | 173 |
| IKSTETGQYLAXDTDGLLYGSQTPNEECLFLERLEENHYNTYISKKHAEK | |
| NWFVGLKKNGSCKRGPRTHYGQKAILFLPLPVSSD | |
| FNLPPGNYKKPKLLYSSNGGHFLRILPDGTVDGTRDRSDQHIQLQLSAESVGEVYIKSTETGQYLCXDT | 174 |
| DGLLYGSQTPNEECLFLERLEENHYNTYISKKHAEKNWFVGLKKNGSVKRGPRTHYGQKAILFLPLPVS | |
| SD | |
| ALTEKFNLPPGNYKKPKLLYSSNGGHFLRILPDGTVDGTRDRSDQHIQLQLSAESVGEVYIKSTETGQY | 175 |
| LCXDTDGLLYGSQTPNEECLFLERLEENHYNTYISKKHAEKNWFVGLKKNGSVKRGPRTHYGQKAILFL | |
| PLPVSSD | |
| LTEKFNLPPGNYKKPKLLYSSNGGHFLRILPDGTVDGTRDRSDQHIQLQLSAESVGEVYIKSTETGQYL | 176 |
| CXDTDGLLYGSQTPNEECLFLERLEENHYNTYISKKHAEKNWFVGLKKNGSVKRGPRTHYGQKAILFLP | |
| LPVSSD | |
| TEKFNLPPGNYKKPKLLYSSNGGHELRILPDGTVDGTRDRSDQHIQLQLSAESVGEVYIKSTETGQYLC | 177 |
| XDTDGLLYGSQTPNEECLFLERLEENHYNTYISKKHAEKNWFVGLKKNGSVKRGPRTHYGQKAILFLPL | |
| PVSSD | |
| EKFNLPPGNYKKPKLLYSSNGGHFLRILPDGTVDGTRDRSDQHIQLQLSAESVGEVYIKSTETGQYLCX | 178 |
| DTDGLLYGSQTPNEECLELERLEENHYNTYISKKHAEKNWFVGLKKNGSVKRGPRTHYGQKAILFLPLP | |
| VSSD | |
| KFNLPPGNYKKPKLLYSSNGGHFLRILPDGTVDGTRDRSDQHIQLQLSAESVGEVYIKSTETGQYLCXD | 179 |
| TDGLLYGSQTPNEECLFLERLEENHYNTYISKKHAEKNWFVGLKKNGSVKRGPRTHYGQKAILFLPLPV | |
| SSD | |
| KFNLPPGNYKKPKLLYSSNGGHELRILPDGTVDGTRDRSDQHIQLQLSAESVGEVYIKSTETGQYLCXD | 180 |
| TDGLLYGSQTPNEECLFLERLEENHYNTYISKKHAEKNWFVGLKKNGSVKRGPRTHYGQKAILFLPLPV | |
| SSD | |
| ALTEKNLPPGNYKKPKLLYSSNGGHFLRILPDGTVDGTRDRSDQHIQLQLSAESVGEVYIKSTETGQYL | 181 |
| CXDTDGLLYGSQTPNEECLFLERLEENHYNTYISKKHAEKNWFVGLKKNGSVKRGPRTHYGQKAILFLP | |
| LPVSSD | |
| ALTEKLPPGNYKKPKLLYSSNGGHFLRILPDGTVDGTRDRSDQHIQLQLSAESVGEVYIKSTETGQYLC | 182 |
| XDTDGLLYGSQTPNEECLFLERLEENHYNTYISKKHAEKNWFVGLKKNGSVKRGPRTHYGQKAILFLPL | |
| PVSSD | |
| ALTEKPPGNYKKPKLLYSSNGGHFLRILPDGTVDGTRDRSDQHIQLQLSAESVGEVYIKSTETGQYLCX | 183 |
| DTDGLLYGSQTPNEECLELERLEENHYNTYISKKHAEKNWFVGLKKNGSVKRGPRTHYGQKAILFLPLP | |
| VSSD | |
| ALTEKPGNYKKPKLLYSSNGGHFLRILPDGTVDGTRDRSDQHIQLQLSAESVGEVYIKSTETGQYLCXD | 184 |
| TDGLLYGSQTPNEECLFLERLEENHYNTYISKKHAEKNWFVGLKKNGSVKRGPRTHYGQKAILFLPLPV | |
| SSD | |
| LTEKFNLPPGNYKKPKLLYSSNGGHFLRILPDGTVDGTRDRSDQHIQLQLSAESVGEVYIKSTETGQYL | 185 |
| CXDTDGLLYGSQTPNEECLFLERLEENHYNTYISKKHAEKNWFVGLKKNGSVKRGPRTHYGQKAILFLP | |
| LPVSSD | |
| LTEKNLPPGNYKKPKLLYSSNGGHFLRILPDGTVDGTRDRSDQHIQLQLSAESVGEVYIKSTETGQYLC | 186 |
| XDTDGLLYGSQTPNEECLFLERLEENHYNTYISKKHAEKNWFVGLKKNGSVKRGPRTHYGQKAILFLPL | |
| PVSSD | |
| LTEKLPPGNYKKPKLLYSSNGGHFLRILPDGTVDGTRDRSDQHIQLQLSAESVGEVYIKSTETGQYLCX | 187 |
| DTDGLLYGSQTPNEECLELERLEENHYNTYISKKHAEKNWFVGLKKNGSVKRGPRTHYGQKAILFLPLP | |
| VSSD | |
| LTEKPPGNYKKPKLLYSSNGGHFLRILPDGTVDGTRDRSDQHIQLQLSAESVGEVYIKSTETGQYLCXD | 188 |
| TDGLLYGSQTPNEECLFLERLEENHYNTYISKKHAEKNWFVGLKKNGSVKRGPRTHYGQKAILFLPLPV | |
| SSD | |
| LTEKPGNYKKPKLLYSSNGGHFLRILPDGTVDGTRDRSDQHIQLQLSAESVGEVYIKSTETGQYLCXDT | 189 |
| DGLLYGSQTPNEECLFLERLEENHYNTYISKKHAEKNWFVGLKKNGSVKRGPRTHYGQKAILFLPLPVS | |
| SD | |
| TEKENLPPGNYKKPKLLYSSNGGHFLRILPDGTVDGTRDRSDQHIQLQLSAESVGEVYIKSTETGQYLC | 190 |
| XDTDGLLYGSQTPNEECLFLERLEENHYNTYISKKHAEKNWFVGLKKNGSVKRGPRTHYGQKAILFLPL | |
| PVSSD | |
| TEKNLPPGNYKKPKLLYSSNGGHELRILPDGTVDGTRDRSDQHIQLQLSAESVGEVYIKSTETGQYLCX | 191 |
| DTDGLLYGSQTPNEECLFLERLEENHYNTYISKKHAEKNWFVGLKKNGSVKRGPRTHYGQKAILELPLP | |
| VSSD | |
| TEKLPPGNYKKPKLLYSSNGGHFLRILPDGTVDGTRDRSDQHIQLQLSAESVGEVYIKSTETGQYLCXD | 192 |
| TDGLLYGSQTPNEECLFLERLEENHYNTYISKKHAEKNWFVGLKKNGSVKRGPRTHYGQKAILELPLPV | |
| SSD | |
| TEKPPGNYKKPKLLYSSNGGHELRILPDGTVDGTRDRSDQHIQLQLSAESVGEVYIKSTETGQYLCXDT | 193 |
| DGLLYGSQTPNEECLFLERLEENHYNTYISKKHAEKNWFVGLKKNGSVKRGPRTHYGQKAILFLPLPVS | |
| SD | |
| TEKPGNYKKPKLLYSSNGGHFLRILPDGTVDGTRDRSDQHIQLQLSAESVGEVYIKSTETGQYLCXDTD | 194 |
| GLLYGSQTPNEECLFLERLEENHYNTYISKKHAEKNWFVGLKKNGSVKRGPRTHYGQKAILFLPLPVSS | |
| D | |
| EKFNLPPGNYKKPKLLYSSNGGHFLRILPDGTVDGTRDRSDQHIQLQLSAESVGEVYIKSTETGQYLCX | 195 |
| DTDGLLYGSQTPNEECLFLERLEENHYNTYISKKHAEKNWFVGLKKNGSVKRGPRTHYGQKAILFLPLP | |
| VSSD | |
| EKNLPPGNYKKPKLLYSSNGGHFLRILPDGTVDGTRDRSDQHIQLQLSAESVGEVYIKSTETGQYLCXD | 196 |
| TDGLLYGSQTPNEECLFLERLEENHYNTYISKKHAEKNWFVGLKKNGSVKRGPRTHYGQKAILFLPLPV | |
| SSD | |
| EKLPPGNYKKPKLLYSSNGGHFLRILPDGTVDGTRDRSDQHIQLQLSAESVGEVYIKSTETGQYLCXDT | 197 |
| DGLLYGSQTPNEECLFLERLEENHYNTYISKKHAEKNWFVGLKKNGSVKRGPRTHYGQKAILFLPLPVS | |
| SD | |
| EKPPGNYKKPKLLYSSNGGHFLRILPDGTVDGTRDRSDQHIQLQLSAESVGEVYIKSTETGQYLCXDTD | 198 |
| GLLYGSQTPNEECLFLERLEENHYNTYISKKHAEKNWFVGLKKNGSVKRGPRTHYGQKAILELPLPVSS | |
| D | |
| EKPGNYKKPKLLYSSNGGHFLRILPDGTVDGTRDRSDQHIQLQLSAESVGEVYIKSTETGQYLCXDTDG | 199 |
| LLYGSQTPNEECLFLERLEENHYNTYISKKHAEKNWFVGLKKNGSVKRGPRTHYGQKAILFLPLPVSSD | |
| KFNLPPGNYKKPKLLYSSNGGHFLRILPDGTVDGTRDRSDQHIQLQLSAESVGEVYIKSTETGQYLCXD | 200 |
| TDGLLYGSQTPNEECLFLERLEENHYNTYISKKHAEKNWFVGLKKNGSVKRGPRTHYGQKAILELPLPV | |
| SSD | |
| KNLPPGNYKKPKLLYSSNGGHFLRILPDGTVDGTRDRSDQHIQLQLSAESVGEVYIKSTETGQYLCXDT | 201 |
| DGLLYGSQTPNEECLFLERLEENHYNTYISKKHAEKNWFVGLKKNGSVKRGPRTHYGQKAILFLPLPVS | |
| SD | |
| KLPPGNYKKPKLLYSSNGGHFLRILPDGTVDGTRDRSDQHIQLQLSAESVGEVYIKSTETGQYLCXDTD | 202 |
| GLLYGSQTPNEECLFLERLEENHYNTYISKKHAEKNWFVGLKKNGSVKRGPRTHYGQKAILFLPLPVSS | |
| D | |
| KPPGNYKKPKLLYSSNGGHFLRILPDGTVDGTRDRSDQHIQLQLSAESVGEVYIKSTETGQYLCXDTDG | 203 |
| LLYGSQTPNEECLFLERLEENHYNTYISKKHAEKNWFVGLKKNGSVKRGPRTHYGQKAILFLPLPVSSD | |

1. A method of treating or preventing a corneal disease or condition in a subject in need thereof,
comprising administering intracamerally to the subject a pharmaceutical composition comprising a therapeutically effective amount of a modified FGF-1 polypeptide having at least 90% sequence identity to the wild-type FGF-1 amino acid sequence of SEQ ID NO: 1 and the mutations of Cys16Ser, Ala66Cys, and Cys117Val,
wherein the corneal disease or condition comprises a procedure of Descemetorhexis without Endothelial Keratoplasty (DWEK) or Descemet Stripping Only (DSO).

2. The method of claim 1, wherein the modified FGF-1 polypeptide comprises a methionine upstream of the first residue of SEQ ID NO: 1.

3. The method of claim 1, wherein the modified FGF-1 polypeptide comprises an amino acid sequence of that is at least about 90% identical to the sequence of SEQ ID NO: 2.

4. (canceled)

5. (canceled)

6. The method of claim 1, wherein the modified FGF-1 polypeptide comprises the amino acid sequence of SEQ ID NO: 2.

7. The method of claim 1, wherein the subject undergoes cataract surgery prior to, currently with, after the administering.

8. The method of claim 1, wherein the corneal disease or condition comprises a corneal endothelial dystrophy or a corneal endothelial damage.

9. The method of claim 1, wherein:
(i) the subject undergoes cataract surgery prior to, currently with, after the administering; and
(ii) the corneal disease or condition comprises a corneal endothelial dystrophy or a corneal endothelial damage.

10. The method of claim 9, wherein the corneal endothelial dystrophy or corneal endothelial damage comprises Fuchs dystrophy, bullous keratopathy, congenital hereditary endothelial dystrophy 1, congenital hereditary endothelial dystrophy 2, posterior polymorphous corneal dystrophy, a corneal endothelial damage secondary to ocular surgery, a corneal endothelial damage secondary to cataract surgery, or a corneal endothelial damage secondary to oxidative stress.

11. (canceled)

12. (canceled)

13. (canceled)

14. The method of claim 7, wherein the DWEK or DSO is performed prior to, concurrently with, or after the cataract surgery.

15.-37. (canceled)

38. The method of claim 1, wherein the administering comprises administering a dose of at least about 10 ng of the modified FGF-1 polypeptide.

39. The method of claim 1, wherein the administering comprises administering a dose of at least about 50 ng of the modified FGF-1 polypeptide.

40. The method of claim 1, wherein the administering comprises administering a dose of at least about 100 ng of the modified FGF-1 polypeptide.

41. The method of claim 1, wherein the administering comprises administering a first dose and a second dose, wherein the first or second dose is at least about 10 ng of the modified FGF-1 polypeptide.

42.-47. (canceled)

48. The method of claim 1, wherein the administering comprises administering a dose of the modified FGF-1 polypeptide before, concurrently with, or after the procedure of DWEK or DSO.

49. (canceled)

50. (canceled)

51. (canceled)

52. The method of claim 1, wherein the administering comprises administering a dose of the modified FGF-1 polypeptide on the same day of the procedure of DWEK or DSO.

53-57. (canceled)

58. The method of claim 1, wherein the administering comprises administering a dose of the modified FGF-1 polypeptide on the 7^{th}, 14^{th}, 21^{st} day after the procedure of DWEK or DSO.

59.-71. (canceled)

72. The method of claim 1, wherein the administering results in a therapeutic effect comprising one or more selected from the group comprising a reduction in corneal endothelial damage, an increase in migration of corneal endothelial cell, an increase in proliferation of corneal endothelial cell, a reduction in corneal edema, an improvement in Best-Corrected Visual Acuity (BCVA), a decrease in corneal thickness, a decrease in central corneal thickness, an improvement in the measurement of visual function and corneal health status (V-FUCHS), a reduction in intraocular pressure, an improvement of slit lamp examination, an improvement in central corneal endothelial cell count, an improvement in peripheral corneal endothelial cell count, or a reduction in time of achieving any thereof.

73. (canceled)

74. The method of claim 1, wherein the administering results in a therapeutic effect within at most 2 weeks after the administering.

75.-81. (canceled)

82. The method of claim 1, wherein the administering results in a therapeutic effect in the subject with or without cataract surgery, wherein the therapeutic effect is similar or same between a subject with cataract surgery and a subject without cataract surgery.

83. The method of claim 1, wherein the administering results in at least one of the following: (i) an improvement in corneal edema or central corneal thickness in the subject with or without cataract surgery, wherein the improvement in corneal edema or central corneal thickness is similar or same between a subject with cataract surgery and a subject without cataract surgery; (ii) an improvement in BCVA; (iii) a reduction or elimination of corneal edema; (iv) an accelerated reduction or elimination of corneal edema; and (v) a reduction in corneal thickness.

84.-99. (canceled)
